SEQUENCE LISTING (8 sequences)

SEQ ID NO: 1 - protein, 563 aa, Homo sapiens
  TPP1 protein sequence (UniProtKB/Swiss-Prot Accession No. O14773-1)
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 2 - DNA, 1693 bp, Homo sapiens
  Native TPP1 Coding Sequence
atgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctgac

SEQ ID NO: 3 - DNA, 1693 bp, Artificial Sequence
  Codon-optimized TPP1 coding sequence
atgggactgcaggcctgtctgctgggactgttcgccctgatcctgagcggcaagtgcagctacagccccgagcccgaccagagaagaacactgcctccaggctgggtgtccctgggcagagctgaccctgaagaggaactgagcctgaccttcgccctgcggcagcagaacgtggaaagactgagcgagctggtgcaggccgtgtccgatcctagcagccctcagtacggcaagtacctgaccctggaaaacgtggccgacctcgtgcggcctagccctctgacactgcacaccgtgcagaagtggctgctggctgccggcgctcagaaatgccactccgtgatcacccaggactttctgacctgttggctgagcatccggcaggccgaactgctgctgcctggggccgagtttcaccactatgtgggcggacccaccgagacacatgtcgtgcgcagcccacacccttaccagctgccacaggctctggcccctcacgtggactttgtgggaggcctgcacagattccccccaaccagcagcctgagacagaggcctgagccacaagtgaccggcacagtgggcctgcatctgggcgtgacacctagcgtgatccggaagcggtacaacctgaccagccaggatgtgggcagcggcaccagcaacaatagccaggcctgcgcccagttcctggaacagtacttccacgacagcgatctggcccagttcatgcggctgttcggcggcaacttcgcacatcaggctagcgtggccagagtcgtgggccagcagggaagaggcagagccggaattgaggcctccctggacgtgcagtacctgatgagcgctggcgccaacatcagcacctgggtgtacagcagccccggcagacacgagggccaggaaccttttctgcagtggctgatgctgctgagcaacgagagcgccctgcctcatgtgcacacagtgtcctacggcgacgacgaggacagcctgagcagcgcctacatccagagagtgaacaccgagctgatgaaggccgctgccaggggactgaccctgctgtttgcctctggcgatagcggagccggctgttggagtgtgtcaggccggcaccagttcagacccacctttcctgccagctccccctacgtgacaaccgtgggcggcacctcctttcaggaacccttcctgatcaccaacgagatcgtggactacatcagcggcggaggcttcagcaacgtgttccccagacccagctaccaggaagaggccgtgaccaagttcctgtcctccagccctcatctgccccccagctcctacttcaacgccagcggcagagcctacccagatgtggccgctctgtccgacggctactgggtggtgtccaacagagtgcccatcccttgggtgtccggcacaagcgccagcacccctgtgtttggcggcatcctgtccctgatcaacgagcacagaatcctgtccggcagaccccccctgggcttcctgaaccctagactgtaccagcagcacggcgctggcctgttcgatgtgaccagaggctgccacgagagctgcctggacgaggaagtggaaggccagggcttctgttctggccctggctgggatcctgtgaccggatggggcacccctaacttccccgccctgctgaaaacactgctgaacccctgat

SEQ ID NO: 4 - protein, 320 aa, Homo sapiens
  TPP1 protein sequence (UniProtKB/Swiss-Prot Accession No. O14773-2)
MRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 5 - DNA, 6841 bp, Artificial Sequence
  Exemplary production plasmid which includes the TPP1 encoding sequence
tcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgccaccatgggactgcaggcctgtctgctgggactgttcgccctgatcctgagcggcaagtgcagctacagccccgagcccgaccagagaagaacactgcctccaggctgggtgtccctgggcagagctgaccctgaagaggaactgagcctgaccttcgccctgcggcagcagaacgtggaaagactgagcgagctggtgcaggccgtgtccgatcctagcagccctcagtacggcaagtacctgaccctggaaaacgtggccgacctcgtgcggcctagccctctgacactgcacaccgtgcagaagtggctgctggctgccggcgctcagaaatgccactccgtgatcacccaggactttctgacctgttggctgagcatccggcaggccgaactgctgctgcctggggccgagtttcaccactatgtgggcggacccaccgagacacatgtcgtgcgcagcccacacccttaccagctgccacaggctctggcccctcacgtggactttgtgggaggcctgcacagattccccccaaccagcagcctgagacagaggcctgagccacaagtgaccggcacagtgggcctgcatctgggcgtgacacctagcgtgatccggaagcggtacaacctgaccagccaggatgtgggcagcggcaccagcaacaatagccaggcctgcgcccagttcctggaacagtacttccacgacagcgatctggcccagttcatgcggctgttcggcggcaacttcgcacatcaggctagcgtggccagagtcgtgggccagcagggaagaggcagagccggaattgaggcctccctggacgtgcagtacctgatgagcgctggcgccaacatcagcacctgggtgtacagcagccccggcagacacgagggccaggaaccttttctgcagtggctgatgctgctgagcaacgagagcgccctgcctcatgtgcacacagtgtcctacggcgacgacgaggacagcctgagcagcgcctacatccagagagtgaacaccgagctgatgaaggccgctgccaggggactgaccctgctgtttgcctctggcgatagcggagccggctgttggagtgtgtcaggccggcaccagttcagacccacctttcctgccagctccccctacgtgacaaccgtgggcggcacctcctttcaggaacccttcctgatcaccaacgagatcgtggactacatcagcggcggaggcttcagcaacgtgttccccagacccagctaccaggaagaggccgtgaccaagttcctgtcctccagccctcatctgccccccagctcctacttcaacgccagcggcagagcctacccagatgtggccgctctgtccgacggctactgggtggtgtccaacagagtgcccatcccttgggtgtccggcacaagcgccagcacccctgtgtttggcggcatcctgtccctgatcaacgagcacagaatcctgtccggcagaccccccctgggcttcctgaaccctagactgtaccagcagcacggcgctggcctgttcgatgtgaccagaggctgccacgagagctgcctggacgaggaagtggaaggccagggcttctgttctggccctggctgggatcctgtgaccggatggggcacccctaacttccccgccctgctgaaaacactgctgaacccctgatgac

SEQ ID NO: 6 - protein, 736 aa, Adeno-Associated Virus 9 (AAV9)
  AAV9 Capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - DNA, 2211 bp, Adeno-Associated Virus 9 (AAV9)
  AAV9 Capsid coding sequence
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 8 - DNA, 4020 bp, Artificial Sequence
  Exemplary sequence of an expression cassette
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgccaccatgggactgcaggcctgtctgctgggactgttcgccctgatcctgagcggcaagtgcagctacagccccgagcccgaccagagaagaacactgcctccaggctgggtgtccctgggcagagctgaccctgaagaggaactgagcctgaccttcgccctgcggcagcagaacgtggaaagactgagcgagctggtgcaggccgtgtccgatcctagcagccctcagtacggcaagtacctgaccctggaaaacgtggccgacctcgtgcggcctagccctctgacactgcacaccgtgcagaagtggctgctggctgccggcgctcagaaatgccactccgtgatcacccaggactttctgacctgttggctgagcatccggcaggccgaactgctgctgcctggggccgagtttcaccactatgtgggcggacccaccgagacacatgtcgtgcgcagcccacacccttaccagctgccacaggctctggcccctcacgtggactttgtgggaggcctgcacagattccccccaaccagcagcctgagacagaggcctgagccacaagtgaccggcacagtgggcctgcatctgggcgtgacacctagcgtgatccggaagcggtacaacctgaccagccaggatgtgggcagcggcaccagcaacaatagccaggcctgcgcccagttcctggaacagtacttccacgacagcgatctggcccagttcatgcggctgttcggcggcaacttcgcacatcaggctagcgtggccagagtcgtgggccagcagggaagaggcagagccggaattgaggcctccctggacgtgcagtacctgatgagcgctggcgccaacatcagcacctgggtgtacagcagccccggcagacacgagggccaggaaccttttctgcagtggctgatgctgctgagcaacgagagcgccctgcctcatgtgcacacagtgtcctacggcgacgacgaggacagcctgagcagcgcctacatccagagagtgaacaccgagctgatgaaggccgctgccaggggactgaccctgctgtttgcctctggcgatagcggagccggctgttggagtgtgtcaggccggcaccagttcagacccacctttcctgccagctccccctacgtgacaaccgtgggcggcacctcctttcaggaacccttcctgatcaccaacgagatcgtggactacatcagcggcggaggcttcagcaacgtgttccccagacccagctaccaggaagaggccgtgaccaagttcctgtcctccagccctcatctgccccccagctcctacttcaacgccagcggcagagcctacccagatgtggccgctctgtccgacggctactgggtggtgtccaacagagtgcccatcccttgggtgtccggcacaagcgccagcacccctgtgtttggcggcatcctgtccctgatcaacgagcacagaatcctgtccggcagaccccccctgggcttcctgaaccctagactgtaccagcagcacggcgctggcctgttcgatgtgaccagaggctgccacgagagctgcctggacgaggaagtggaaggccagggcttctgttctggccctggctgggatcctgtgaccggatggggcacccctaacttccccgccctgctgaaaacactgctgaacccctgatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

(19) United States
(12) Patent Application Publication
(10) Pub. No.: US 2024/0091380 A1
(43) Pub. Date: Mar. 21, 2024

(54) GENE THERAPY FOR NEURONAL CEROID LIPOFUSCINOSES

(71) Applicant: REGENXBIO Inc., Rockville, MD (US)

(72) Inventors: Marie-Laure Nevoret, Virginia Beach, VA (US); Nicholas Alexander Piers Sascha Buss, Rockville, MD (US); Paulo Falabella, Potomac, MD (US); Stephen Joseph Pakola, Irvington, NY (US)

(21) Appl. No.: 18/274,499

(22) PCT Filed: Jan. 31, 2022
(86) PCT No.: PCT/US2022/014520

§ 371 Date: Jul. 27, 2023

Related U.S. Application Data
(60) Provisional application No. 63/252,746, filed on Oct. 6, 2021.
(60) Provisional application No. 63/144,252, filed on Feb. 1, 2021.

(51) Int. Cl. A61K 48/00; A61K 9/00; A61K 45/06; A61P 25/28; C12N 9/48; C12N 15/86

(52) U.S. Cl. CPC A61K 48/005; A61K 9/0085; A61K 45/06; A61K 48/0075; A61P 25/28; C12N 9/485; C12N 15/86; C12N 2750/14143; C12Y 304/14009

(57) ABSTRACT

Provided herein are methods and compositions for treatment of CLN2 Disease. Such compositions include a recombinant adeno-associated virus (rAAV), said rAAV comprising an AAV capsid, and a vector genome packaged therein, said vector genome comprising (a) an AAV 5′ inverted terminal repeat (ITR) sequence; (b) a promoter; (c) a CLN2 coding sequence encoding a human TPP1; (d) an AAV 3′ ITR. Also provided herein are methods of treating CLN2 Disease comprising administering to a subject in need thereof the rAAV described herein via more than one route. Also provide herein are pharmaceutical compositions comprising the rAAV described herein and related methods of treating CLN2 Disease.

### 1. PRIORITY

This application claims the benefit of priority to U.S. Ser. No. 63/144,252 filed Feb. 1, 2021 and U.S. Ser. No. 63/252,746 filed Oct. 6, 2021, each of which is incorporated herein by reference in its entirety.

### 2. REFERENCE TO SEQUENCE LISTING SUBMITTED ELECTRONICALLY

This application incorporates by reference a Sequence Listing submitted with this application as a text file entitled “12656-153-228_Sequence_Listing.txt” created on Jan. 31, 2022 and having a size of 36,807 bytes.

### 3. BACKGROUND OF THE INVENTION

The neuronal ceroid lipofuscinoses (NCLs) are a group of rare and inherited neurodegenerative disorders. They are considered the most common of the neurogenetic storage diseases, with the accumulation of autofluorescent lipopigments resembling ceroid and lipofuscin seen in patients. NCLs are associated with variable, yet progressive, symptoms, including abnormally increased muscle tone or spasm, blindness or vision problems, dementia, lack of muscle coordination, intellectual disability, movement disorder, seizures and unsteady walk. The frequency of this disease is approximately 1 per 12,500 individuals. There are three main types of NCL: adult (Kufs or Parry disease); juvenile and late infantile (Jansky-Bielschowsky disease). The neuronal ceroid lipofuscinoses (NCLs) originally were defined by their age of onset and clinical symptoms (as noted herein). However, they have since been reclassified on the basis of newer molecular findings, which have provided evidence of far more overlap for the different genetic variants than had previously been suggested by the clinical phenotypes.

At least twenty genes have been identified in association with NCL. NCL patients with CLN2 mutations are deficient in a pepstatin-insensitive lysosomal peptidase called tripeptidyl peptidase 1 (TPP1). TPP1 removes tripeptides from the N-terminal of polypeptides. Mutations have been reported in all 13 exons of the CLN2 gene. Some mutations result in a more protracted course. Although onset is usually in late infancy, later onset has been described. More than 58 mutations have been described in CLN2.

CLN2 disease, a form of CLN2 Disease, is a rare lysosomal storage disorder (LSD) with an estimated incidence of 0.07-0.51 per 100,000 live births (Augestad et al., 2006; Claussen et al., 1992; Mole et al., 2013; National CLN2 Disease Registry; Poupetova et al., 2010; Santorelli et al., 2013; Teixeira et al., 2003). CLN2 disease is a fatal autosomal recessive neurodegenerative LSD caused by mutations in the CLN2 gene, located on chromosome 11q15 and encoding for the soluble lysosomal enzyme tripeptidyl-peptidase-1 (TPP1). Mutations in the CLN2 gene, and subsequent deficiency in TPP1 enzymatic activity, result in lysosomal accumulation of storage material and neurodegeneration of the brain and retina (Liu et al., 1998; Wlodawer et al., 2003). CLN2 disease is characterized by early onset at 2-4 years of age with initial features usually including recurrent seizures (epilepsy) and difficulty coordinating movements (ataxia). The disease also results in the loss of previously acquired skills (developmental regression). Epilepsy is often refractory to medical therapy, and the general decay of psychomotor functions is rapid and uniform between the third and fifth birthday (Schulz et al., 2013) before premature death by mid-childhood (Nickel M et al., 2016; Worgall et al., 2007).

Enzyme replacement therapy (ERT) with recombinant TPP1 (Brineura® cerliponase alfa, BioMarin Pharmaceuticals) was recently approved in the United States (US) and European Union (EU) for the treatment of CLN2 disease and is administered as a biweekly infusion into the lateral ventricles via a permanently implanted device. The clinical benefit of Brineura® was designated to be limited to stabilization of motor function by the FDA, while the European Medicines Agency (EMA) determined that there was a positive impact on language skills as well (Brineura®, FDA Summary Basis of Approval; Brineura® European Public Assessment Report [EPAR]; Schulz et al., 2016). Brineura® requires specialized expertise for the implantation of a port directly into the brain and must be administered during a 4-hour infusion every two weeks in a healthcare setting by a trained professional knowledgeable in intracerebroventricular (ICV) administration. Repeat infusions are necessary in part due to the short CSF and lysosomal half-lives of Brineura® which are estimated to be 7 hours and 11.5 days, respectively (Brineura®, EPAR). Thus, there remains a significant unmet need for new therapies that can provide durable and long-term TPP1 enzymatic activity in the central nervous system (CNS) of patients with CLN2 disease, without the high patient burden and morbidities associated with repeat administration of ERT. Therefore, compositions useful for delivering and expressing TPP1 in subjects in need for treating CLN2 disease are needed. A one-time administration of recombinant adeno-associated virus (rAAV) expressing canine TPP1 (rAAV2.caTPP1) was shown to result in high expression of TPP1 predominantly in ependymal cells and secretion of the enzyme into the cerebrospinal fluid leading to clinical benefit. See Katz et al, Sci Transl Med. 2015 Nov. 11; 7(313): 313 ra180; and KATZ, et al, Gene therapy 2017 Feb. 24(4): 215-223, which are incorporated herein by reference. However AAV2 does not penetrate the brain parenchyma and does not target neurons, thus limiting the expected benefits compared to what can be achieved with novel neurotropic AAVs.

### 4. SUMMARY OF THE INVENTION

Provided herein is a method of treating CLN2 due to TPP1 deficiency in a subject comprising administering to the central nervous system of the subject in need thereof 1.25×10¹¹ or 4.5×10¹¹ genome copies per gram brain mass of a recombinant adeno-associated virus (rAAV) into the central nervous system (CNS), wherein said recombinant adeno-associated virus (rAAV) comprises an AAV capsid and a vector genome packaged therein, and wherein said vector genome comprises (a) an AAV 5′ inverted terminal repeat (ITR) sequence; (b) a promoter; (c) a CLN2 coding sequence encoding a human TPP1; and (d) an AAV 3′ ITR, wherein the method results in an improvement of symptoms of CLN2 disease. In some embodiments the improvement of symptoms of CLN2 disease comprises a less than 2-category decline in the 6-point combined Motor and Language domains of the CLN2 Clinical Rating Scale within 24 months after administration.

In some embodiments, the rAAV is administered intracerebroventricularly (ICV) or intracisternally (IC). In some embodiments, the brain mass of the subject is derived from the study participant's screening brain MRI.

In some embodiments, the coding sequence of (c) is a codon optimized human CLN2 set forth in SEQ ID NO: 3. In some embodiments, the coding sequence of (c) is SEQ ID NO: 3. In some embodiments, n the rAAV capsid is an AAV9 or a variant thereof. In some embodiments, the promoter is a chicken beta actin (CBA) promoter. In some embodiments, the promoter is a hybrid promoter comprising a CBA promoter sequence and cytomegalovirus enhancer elements. In some embodiments, the AAV 5′ ITR and/or AAV3′ ITR is from AAV2.

In some embodiments, the vector genome further comprises a polyA. In some embodiments, the polyA is a synthetic polyA or from bovine growth hormone (bGH), human growth hormone (hGH), SV40, rabbit β-globin (RGB), or modified RGB (mRGB).

In some embodiments, the vector genome further comprises an intron. In some embodiments, the intron is from CBA, human beta globin, IVS2, SV40, bGH, alpha-globulin, beta-globulin, collagen, ovalbumin, or p53.

In some embodiments, the vector genome further comprises an enhancer. In some embodiments, the enhancer is a CMV enhancer, an RSV enhancer, an APB enhancer, ABPS enhancer, an alpha mic/bik enhancer, TTR enhancer, en34, ApoE.

In some embodiments, the method results in a TPP1 activity in the cerebral spinal fluid of the subject that is at least about 50%, at least about 75%, at least about 80%, at least about 90%, or about the same, or greater than 100% of the biological activity level of the native TPP1 protein, or a natural variant or polymorph thereof which is not associated with disease. In some embodiments, the method results in a serum TPP1 activity of said subject that is at least about 50%, at least about 75%, at least about 80%, at least about 90%, or about the same, or greater than 100% of the biological activity level of the native TPP1 protein, or a natural variant or polymorph thereof which is not associated with disease.

In some embodiments, the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the combined Motor and Language domains of the CLN2 CRS. In some embodiments, the e method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the Language domains of the CLN2 CRS. In some embodiments, the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the Motor domains of the CLN2 CRS.

In some embodiments, the method results in a reduction in the frequency of seizures of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as recorded in the Caregiver Seizure Diary. In some embodiments, the method results in a reduction in the duration of seizures of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as recorded in the Caregiver Seizure Diary.

In some embodiments, the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the Pediatric Quality of Life Inventory (PedsQL) Generic Core Scale. In some embodiments, the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the PedsQL Family Impact Module.

In some embodiments, the method results in a decrease in the use of antiepileptic treatments of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline.

In some embodiments, the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by the Vineland Adaptive Behavior Scale III.

In some embodiments, the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by the Mullen Scale of Early Learning.

In some embodiments, the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by assessing retinal anatomy using Spectral Domain Optical Coherence Tomography (SD-OCT).

In some embodiments, the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by Clinician Global Impression of Severity. In some embodiments, the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by Clinician Global Impression of Change.

In some embodiments, the method results in an improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% in gait parameters compared to baseline as determined by GAITRite.

In some embodiments, the method further comprises administering immunosuppressive therapy to the subject. In some embodiments, the immunosuppressive therapy comprises administering corticosteroids, tacrolimus, and/or sirolimus.

In some embodiments, the subject is human. In some embodiments, the subject is between 4 months and 6 years of age. In some embodiments, the subject has a documented diagnosis of CLN2 disease due to TPP1 deficiency, confirmed by biochemical, molecular, or genetic methods.

### 5. BRIEF DESCRIPTION OF THE DRAWINGS

FIG. 1 is a schematic representation of the AAV.CB7.CI.hTPP1co.RBG vector genome. ITR represents an AAV2 inverted terminal repeat. CB7 represents a chicken beta actin promoter with cytomegalovirus enhancer. RBG PolyA represents a rabbit beta globin polyadenylation signal.

FIG. 2 provides a map of the production plasmid of the AAV.hTPP1co vector.

FIG. 3 provides a map of the AAV cis plasmid construct. ITR: inverted terminal repeat; CMV IE promoter: cytomegalovirus immediate-early promoter; CB promoter: chicken 3-actin promoter Chicken 3-actin intron; hCLN2: Human CLN2 cDNA; Rabbit globin poly A: Rabbit beta-globin polyadenylation signal; Kan-r: kanamycin resistance gene.

FIG. 4 provides a map of the AAV trans packaging plasmid construct.

FIG. 5 provides a map of the adenovirus helper plasmid.

FIG. 6 shows Biodistribution of Construct III in the deep and superficial brain regions of cynomolgus monkeys.

FIG. 7 shows Biodistribution of Construct III in the deep and superficial brain regions of cynomolgus monkeys.

FIG. 8 provides a CLN2 CRS-MX scoring flowchart.

FIG. 9 provides a CLN2 CRS-LX scoring flowchart for 2 to <3 Year old subjects.

FIG. 10 shows Construct III increased TPP1 concentration in the (A) serum and (B) CSF of non-human primates.

FIG. 11 shows Construct III led to dose-dependent increase in TPP1 concentration in (A) brain superficial samples and (B) deep brain samples of non-human primates.

FIG. 12 shows Construct III increased hTPP1 activity in the brain of TPP1m1J KO (A) male and (B) female mice (*p<0.05; **p<0.01. P-values are obtained using the 2-sided exact Wilcoxon rank-sum test, comparing each dosed group against an independent control group, with the null hypothesis of no difference between the two groups).

FIG. 13 shows Construct III increased lifespan of TPP1m1J KO (A) male and (B) female mice.

FIG. 14 shows Construct III decreased astrocytosis, microglial activation and SCMAS in the thalamus (S1BF) and cortex (VPM/VPL) of TPP1m1J KO mice after 9 weeks (**p=0.01, ***p-0.001, ****p<0.0001 vs WT; *p<0.05, **p<0.01, ***p<0.001, ****p<0.0001 vs vehicle treated TPP1^{m1J} KO, one way ANOVA, post hoc Bonferroni).

### 6. DETAILED DESCRIPTION OF THE INVENTION

Provided herein are methods and compositions for treatment of CLN2 Disease. Such compositions include a recombinant adeno-associated virus (rAAV), said rAAV comprising an AAV capsid, and a vector genome packaged therein, said vector genome comprising (a) an AAV 5′ inverted terminal repeat (ITR) sequence; (b) a promoter; (c) a CLN2 coding sequence encoding a human TPP1; (d) an AAV 3′ ITR (see Section 6.1). Also described herein are pharmaceutical compositions comprising the rAAV provided herein which may be used to treat CLN2 Disease (see Section 6.2) and methods of treating CLN2 Disease using the rAAV or the compositions provided herein (see Section 6.3).

### 6.1 Recombinant Adeno-Associated Virus (rAAV)

In certain embodiments, the AAV9.CB7.hCLN2 provided herein is described in the following embodiments. The methods and compositions described herein involve compositions and methods for delivering a CLN2 nucleic acid sequence encoding human tripeptidyl peptidase 1 (TPP1) protein to subjects in need thereof for the treatment of NCL. In one embodiment, such compositions involve codon optimization of the CLN2 coding sequence, such as that shown in SEQ ID NO: 3. It is desirable to increase the efficacy of the product, and thus, increase safety, since a lower dose of reagent may be used. Also encompassed herein are compositions which include the native CLN2 coding sequences, as shown in SEQ ID NO: 2.

The TPP1 gene, also known as CLN2, encodes Tripeptidyl-peptidase 1, a lysosomal serine protease with tripeptidyl-peptidase 1 activity. It is also thought to act as a non-specific lysosomal peptidase which generates tripeptides from the breakdown products produced by lysosomal proteinases and requires substrates with an unsubstituted N-terminus. As used herein, the terms “TPP1”, “CLN2”, and “Tripeptidyl-peptidase 1” are used interchangeably when referring to the coding sequence. The native nucleic acid sequence encoding human Tripeptidyl-peptidase 1 is reported at NCBI Reference Sequence NM_000391.3 and reproduced here in SEQ ID NO: 2. Two isoforms of human Tripeptidyl-peptidase 1 has been reported as UniProtKB/Swiss-Prot Accessions 014773-1 and 014773-2 (reproduced here as SEQ ID NOs: 1 and 4). Mutations in the CLN2 gene are associated with late-infantile NCL (LINCL) disease.

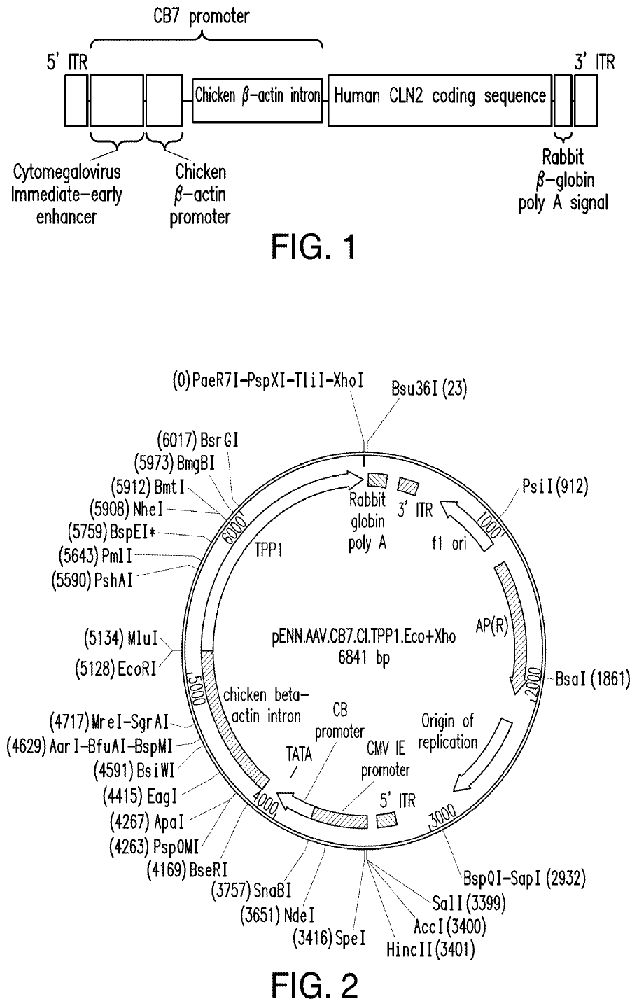

In certain embodiments, AAV.hTPP1co vectors may be designed as described in WO 2018209205A1. In certain embodiments, the human (h) TPP1-endcoding optimized cDNA may be custom-designed for optimal codon usage and synthesized. In certain embodiments, the hTPP1co cDNA as reproduced as SEQ ID NO: 3 may be then placed in a transgene expression cassette which was driven by a CB7 promoter, a hybrid between a cytomegalovirus (CMV) immediate early enhancer (C4) and the chicken beta actin promoter, while transcription from this promoter is enhanced by the presence of the chicken beta actin intron (CI) (FIG. 1 and FIG. 2). In certain embodiments, the polyA signal for the expression cassette is the rabbit beta-globin (RBG) polyA.

In certain embodiments, a 6841 bp production plasmid of AAV.hTPP1co vector (AAV.CB7.CI.hTPP1co.RBG) may be constructed with the hTPP1co expression cassette described herein flanked by AAV2 derived ITRs as well as resistance to Ampicillin as a selective marker (FIG. 2). In certain embodiments, a similar AAV.hTPP1co production plasmid with resistance to Kanamycin may also be constructed. In certain embodiments, the vectors derived from both plasmids may be single-stranded DNA genome with AAV2 derived ITRs flanking the hTPP1co expression cassette described herein.

In certain embodiments, the AAV.hTPP1co vectors may be made by triple transfection and formulated in excipient consisting of phosphate-buffered saline (PBS) containing and 0.001% Pluronic F68 (PF68). See, e.g. Mizukami, Hiroaki, et al., A Protocol for AAV vector production and purification, Diss. Di-vision of Genetic Therapeutics, Center for Molecular Medicine, 1998. In certain embodiments, the genome titers of the vector produced may be determined via droplet digital PCR (ddPCR). See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

Described herein is an exemplary AAV.hTPP1co vector, which is sometimes referred to herein as AAV9.CB7.hCLN2. The use of these terms is interchangeable. In addition, where, in one embodiment, the AAV9.CB7.hCLN2 vector is referred to, alternate embodiments are contemplated utilizing the components as described herein.

In one aspect, a codon optimized, engineered nucleic acid sequence encoding human (h) TPP1 is provided. In certain embodiments, an engineered human (h) TPP1 cDNA is provided herein (as SEQ ID NO: 3), which was designed to maximize translation as compared to the native TPP1 sequence (SEQ ID NO: 2). Preferably, the codon optimized TPP1 coding sequence has less than about 80% identity, preferably about 75% identity or less to the full-length native TPP1 coding sequence (SEQ ID NO: 2). In one embodiment, the codon optimized TPP1 coding sequence has about 74% identity with the native TPP1 coding sequence of SEQ ID NO: 2. In one embodiment, the codon optimized TPP1 coding sequence has about 70% identity with the native TPP1 coding sequence of SEQ ID NO: 2. In one embodiment, the codon optimized TPP1 coding sequence is characterized by improved translation rate as compared to native TPP1 following AAV-mediated delivery (e.g., rAAV). In one embodiment, the codon optimized TPP1 coding sequence shares less than about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or less identity to the full length native TPP1 coding sequence of SEQ ID NO: 2. In one embodiment, the codon optimized nucleic acid sequence is a variant of SEQ ID NO: 3. In another embodiment, the codon optimized nucleic acid sequence a sequence sharing about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or greater identity with SEQ ID NO: 3. In one embodiment, the codon optimized nucleic acid sequence is SEQ ID NO: 3. In another embodiment, the nucleic acid sequence is codon optimized for expression in humans. In other embodiments, a different TPP1 coding sequence is selected.

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Identity may be determined by preparing an alignment of the sequences and through the use of a variety of algorithms and/or computer programs known in the art or commercially available [e.g., BLAST, ExPASy; ClustalO; FASTA; using, e.g., Needleman-Wunsch algorithm, Smith-Waterman algorithm]. Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal Omega”, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

Codon-optimized coding regions can be designed by various different methods. This optimization may be performed using methods which are available on-line (e.g., GeneArt), published methods, or a company which provides codon optimizing services, e.g., DNA2.0 (Menlo Park, CA). One codon optimizing method is described, e.g., in US International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

By “engineered” is meant that the nucleic acid sequences encoding the TPP1 protein described herein are assembled and placed into any suitable genetic element, e.g., naked DNA, phage, transposon, cosmid, episome, etc., which transfers the TPP1 sequences carried thereon to a host cell, e.g., for generating non-viral delivery systems (e.g., RNA-based systems, naked DNA, or the like) or for generating viral vectors in a packaging host cell and/or for delivery to a host cells in a subject. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

As used herein, the term “host cell” may refer to the packaging cell line in which a recombinant AAV is produced from a production plasmid. In the alternative, the term “host cell” may refer to any target cell in which expression of the coding sequence is desired. Thus, a “host cell,” refers to a prokaryotic or eukaryotic cell that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to the cells employed to generate and package the viral vector or recombinant virus. In other embodiments herein, the term “host cell” refers to cultures of CNS cells of various mammalian species for in vitro assessment of the compositions described herein. Still in other embodiments, the term “host cell” is intended to reference the brain cells of the subject being treated in vivo for CLN2 Disease. Such host cells include epithelial cells of the CNS including ependyma, the epithelial lining of the brain ventricular system. Other host cells include neurons, astrocytes, oligoedendrocytes, and microglia.

In one embodiment, the nucleic acid sequence encoding TPP1 further comprises a nucleic acid encoding a tag polypeptide covalently linked thereto. The tag polypeptide may be selected from known “epitope tags” including, without limitation, a myc tag polypeptide, a glutathione-S-transferase tag polypeptide, a green fluorescent protein tag polypeptide, a myc-pyruvate kinase tag polypeptide, a His6 tag polypeptide, an influenza virus hemagglutinin tag polypeptide, a flag tag polypeptide, and a maltose binding protein tag polypeptide.

In another aspect, an expression cassette comprising a nucleic acid sequence that encodes TPP1 is provided. In one embodiment, the sequence is a codon optimized sequence. In another embodiment, the codon optimized nucleic acid sequence is SEQ ID NO: 3 encoding human TPP1.

As used herein, an “expression cassette” refers to a nucleic acid molecule which comprises the coding sequences for TPP1 protein, promoter, and may include other regulatory sequences therefor, which cassette may be packaged into the capsid of a viral vector (e.g., a viral particle). Typically, such an expression cassette for generating a viral vector contains the CLN2 sequences described herein flanked by packaging signals of the viral genome and other expression control sequences such as those described herein. For example, for an AAV viral vector, the packaging signals are the 5′ inverted terminal repeat (ITR) and the 3′ ITR. When packaged into the AAV capsid, the ITRs in conjunction with the expression cassette may be referred to herein as the “recombinant AAV (rAAV) genome” or “vector genome”. In one embodiment, an expression cassette comprises a codon optimized nucleic acid sequence that encodes TPP1 protein. In one embodiment, the cassette provides the codon optimized CLN2 operatively associated with expression control sequences that direct expression of the codon optimized nucleic acid sequence that encodes TPP1 in a host cell.

In another embodiment, an expression cassette for use in an AAV vector is provided. In that embodiment, the AAV expression cassette includes at least one AAV inverted terminal repeat (ITR) sequence. In another embodiment, the expression cassette comprises 5′ ITR sequences and 3′ ITR sequences. In one embodiment, the 5′ and 3′ ITRs flank the codon optimized nucleic acid sequence that encodes TPP1, optionally with additional sequences which direct expression of the codon optimized nucleic acid sequence that encodes TPP1 in a host cell. Thus, as described herein, a AAV expression cassette is meant to describe an expression cassette as described above flanked on its 5′ end by a 5′AAV inverted terminal repeat sequence (ITR) and on its 3′ end by a 3′ AAV ITR. Thus, this rAAV genome contains the minimal sequences required to package the expression cassette into an AAV viral particle, i.e., the AAV 5′ and 3′ ITRs. The AAV ITRs may be obtained from the ITR sequences of any AAV, such as described herein. These ITRs may be of the same AAV origin as the capsid employed in the resulting recombinant AAV, or of a different AAV origin (to produce an AAV pseudotype). In one embodiment, the ITR sequences from AAV2, or the deleted version thereof (AITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, the AAV vector genome comprises an AAV 5′ ITR, the TPP1 coding sequences and any regulatory sequences, and an AAV 3′ ITR. However, other configurations of these elements may be suitable. A shortened version of the 5′ ITR, termed AITR, has been described in which the D-sequence and terminal resolution site (trs) are deleted. In other embodiments, the full-length AAV 5′ and 3′ ITRs are used. Each rAAV genome can be then introduced into a production plasmid.

As used herein, the term “regulatory sequences”, “transcriptional control sequence” or “expression control sequence” refers to DNA sequences, such as initiator sequences, enhancer sequences, and promoter sequences, which induce, repress, or otherwise control the transcription of protein encoding nucleic acid sequences to which they are operably linked.

As used herein, the term “operably linked” or “operatively associated” refers to both expression control sequences that are contiguous with the nucleic acid sequence encoding the TPP1 and/or expression control sequences that act in trans or at a distance to control the transcription and expression thereof.

In one aspect, a vector comprising any of the expression cassettes described herein is provided. As described herein, such vectors can be plasmids of variety of origins and are useful in certain embodiments for the generation of recombinant replication defective viruses as described further herein.

A “vector” as used herein is a nucleic acid molecule into which an exogenous or heterologous or engineered nucleic acid transgene may be inserted which can then be introduced into an appropriate host cell. Vectors preferably have one or more origin of replication, and one or more site into which the recombinant DNA can be inserted. Vectors often have means by which cells with vectors can be selected from those without, e.g., they encode drug resistance genes. Common vectors include plasmids, viral genomes, and (primarily in yeast and bacteria) “artificial chromosomes.” Certain plasmids are described herein.

In one embodiment, the vector is a non-viral plasmid that comprises an expression cassette described thereof, e.g., “naked DNA”, “naked plasmid DNA”, RNA, and mRNA; coupled with various compositions and nano particles, including, e.g., micelles, liposomes, cationic lipid—nucleic acid compositions, poly-glycan compositions and other polymers, lipid and/or cholesterol-based—nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference. Such non-viral TPP1 vector may be administered by the routes described herein. The viral vectors, or non-viral vectors, can be formulated with a physiologically acceptable carrier for use in gene transfer and gene therapy applications.

In another embodiment, the vector is a viral vector that comprises an expression cassette described therein. “Virus vectors” are defined as replication defective viruses containing the exogenous or heterologous CLN2 nucleic acid transgene. In one embodiment, an expression cassette as described herein may be engineered onto a plasmid which is used for drug delivery or for production of a viral vector. Suitable viral vectors are preferably replication defective and selected from amongst those which target brain cells. Viral vectors may include any virus suitable for gene therapy, including but not limited to adenovirus; herpes virus; lentivirus; retrovirus; parvovirus, etc. However, for ease of understanding, the adeno-associated virus is referenced herein as an exemplary virus vector.

A “replication-defective virus” or “viral vector” refers to a synthetic or recombinant viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

In another embodiment, a recombinant adeno-associated virus (rAAV) vector is provided. The rAAV compromises an AAV capsid, and a vector genome packaged therein.

The vector genome comprises, in one embodiment: (a) an AAV 5′ inverted terminal repeat (ITR) sequence; (b) a promoter; (c) a coding sequence encoding a human TPP1; and (d) an AAV 3′ ITR. In another embodiment, the vector genome is the expression cassette described herein. In one embodiment, the CLN2 sequence encodes a full length TPP1 protein. In one embodiment, the TPP1 sequence is the protein sequence of SEQ ID NO: 1. In another embodiment, the coding sequence is SEQ ID NO: 3 or a variant thereof.

Adeno-associated virus (AAV), a member of the Parvovirus family, is a small nonenveloped, icosahedral virus with single-stranded linear DNA genomes of 4.7 kilobases (kb) to 6 kb. Among known AAV serotypes are AAV1, AAV2, AAV3, AAV4, AAV5, AAV6, AAV7, AAV8, AAV9 and others. The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, VA). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

Fragments of AAV may be readily utilized in a variety of vector systems and host cells. Among desirable AAV fragments are the cap proteins, including the vp1, vp2, vp3 and hypervariable regions, the rep proteins, including rep 78, rep 68, rep 52, and rep 40, and the sequences encoding these proteins. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a novel AAV sequence of the invention (e.g., a fragment of a vp1 capsid protein) in combination with heterologous sequences which may be obtained from another AAV serotype (known or novel), non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. In one embodiment, a vector contains AAV9 cap and/or rep sequences. See, U.S. Pat. No. 7,906,111, which is incorporated by reference herein.

In one embodiment, an AAV vector having AAV9 capsid characterized by the amino acid sequence of SEQ ID NO: 6, is provided herein, in which a nucleic acid encoding a classic late infantile neuronal ceroid lipofuscinosis 2 (CLN2) gene under control of regulatory sequences directing expression thereof in patients in need thereof.

As used herein, an “AAV9 capsid” is characterized by DNAse-resistant particle which is an assembly of about 60 variable proteins (vp) which are typically expressed as alternative splice variants resulting in proteins of different length of SEQ ID NO: 6. See also Genbank Accession No. AAS99264.1, which is incorporated herein by reference. See, also U.S. Pat. No. 7,906,111 and WO 2005/033321. As used herein “AAV9 variants” include those described in, e.g., WO2016/049230, U.S. Pat. No. 8,927,514, US 2015/0344911, and U.S. Pat. No. 8,734,809. The amino acid sequence is reproduced in SEQ ID NO: 6 and the coding sequence is reproduced in SEQ ID NO: 7. In one embodiment, the AAV9 capsid includes a capsid encoded by SEQ ID NO: 7, or a sequence sharing at least about 90%, 95%, 95%, 98% or 99% identity therewith.

The largest protein, vp1, is generally the full-length of the amino acid sequence of SEQ ID NO: 6 (aa 1-736 of SEQ ID NO: 6). In certain embodiments, the AAV9 vp2 protein has the amino acid sequence of 138 to 736 of SEQ ID NO: 6. In certain embodiments, the AAV9 vp3 has the amino acid sequence of 203 to 736 of SEQ ID NO: 6. In certain embodiments, the vp1, 2 or 3 proteins may be have truncations (e.g., 1 or more amino acids at the N-terminus or C-terminus). An AAV9 capsid is composed of about 60 vp proteins, in which vp1, vp2 and vp3 are present in a ratio of about 1 vp, to about 1 vp2, to about 10 to 20 vp3 proteins within the assembled capsid. This ratio may vary depending upon the production system used. In certain embodiments, an engineered AAV9 capsid may be generated in which vp2 is absent.

It is within the skill in the art to design nucleic acid sequences encoding this AAV9 capsid, including DNA (genomic or cDNA), or RNA (e.g., mRNA). In certain embodiments, the nucleic acid sequence encoding the AAV9 vp1 capsid protein is provided in SEQ ID NO: 7. In other embodiments, a nucleic acid sequence of 70% to 99.9% identity to SEQ ID NO: 7 may be selected to express the AAV9 capsid. In certain other embodiments, the nucleic acid sequence is at least about 75% identical, at least 80% identical, at least 85%, at least 90%, at least 95%, at least 97% identical, or at least 99% to 99.9% identical to SEQ ID NO: 7.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics, Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78(10): 6381-6388, which identifies Clades A, B, C, D, E and F, and provides nucleic acid sequences of novel AAV, GenBank Accession Numbers AY530553 to AY530629. See, also, WO 2005/033321. AAV9 is further characterized by being within Clade F. Other Clade F AAV include AAVhu31 and AAVhu32.

As used herein, relating to AAV, the term variant means any AAV sequence which is derived from a known AAV sequence, including those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV9 capsid. When determining the percent identity of an AAV capsid, the comparison may be made over any of the variable proteins (e.g., vp1, vp2, or vp3). In one embodiment, the AAV capsid shares at least 95% identity with the AAV9 over the vp1, vp2 or vp3.

As used herein, “artificial AAV” means, without limitation, an AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/9 and AAV2/rh.10 are exemplary pseudotyped vectors.

In another embodiment, a self-complementary AAV is used. “Self-complementary AAV” refers a plasmid or vector having an expression cassette in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same host cell or subject, but which is present in a non-natural state, e.g. a different copy number, or under the control of different regulatory elements.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence with which the protein or nucleic acid in question is not found in the same relationship to each other in nature.

In still another embodiment, the expression cassette, including any of those described herein is employed to generate a recombinant AAV genome.

In one embodiment, the expression cassette described herein is engineered into a suitable genetic element (vector) useful for generating viral vectors and/or for delivery to a host cell, e.g., naked DNA, phage, transposon, cosmid, episome, etc., which transfers the CLN2 sequences carried thereon. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY.

For packaging an expression cassette or rAAV genome or production plasmid into virions, the ITRs are the only AAV components required in cis in the same construct as the expression cassette. In one embodiment, the coding sequences for the replication (rep) and/or capsid (cap) are removed from the AAV genome and supplied in trans or by a packaging cell line in order to generate the AAV vector.

Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In a specific embodiment, the producer cell line or packaging cell line is a suspension cell line such that the AAV viral vectors described herein can be manufactured by growing the producer cell line or packaging cell line in suspension culture. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

In yet another system, the expression cassette flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety.

In one aspect, provided herein is a method of manufacturing an rAAV described herein, comprising growing in suspension culture a suspension cell line that is capable of producing the rAAV.

In certain embodiments, the suspension cell line is derived from an adherent cell line by adaptation of cells into suspension culture using serum-free and animal component-free culture medium. In a specific embodiment, the suspension cell line is HEK293 suspension cell line.

The methods used to construct any embodiment of this invention are known to those with skill in nucleic acid manipulation and include genetic engineering, recombinant engineering, and synthetic techniques. See, e.g., Green and Sambrook et al, Molecular Cloning: A Laboratory Manual, Cold Spring Harbor Press, Cold Spring Harbor, NY (2012). Similarly, methods of generating rAAV virions are well known and the selection of a suitable method is not a limitation on the present invention. See, e.g., K. Fisher et al, (1993) J. Virol., 70:520-532 and U.S. Pat. No. 5,478,745.

“Plasmids” generally are designated herein by a lower case p preceded and/or followed by capital letters and/or numbers, in accordance with standard naming conventions that are familiar to those of skill in the art. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

In one embodiment, the production plasmid is that described herein, or as described in WO2012/158757, which is incorporated herein by reference. Various plasmids are known in the art for use in producing rAAV vectors, and are useful herein. The production plasmids are cultured in the host cells which express the AAV cap and/or rep proteins. In the host cells, each rAAV genome is rescued and packaged into the capsid protein or envelope protein to form an infectious viral particle.

In one aspect, a production plasmid comprising an expression cassette described above is provided. In one embodiment, the production plasmid is that shown in FIG. 2. This plasmid is used in the examples for generation of the rAAV-human codon optimized TPP1 vector. Such a plasmid is one that contains a 5′ AAV ITR sequence; a selected promoter; a polyA sequence; and a 3′ ITR; additionally, it also contains an intron sequence, such as the chicken beta-actin intron. An exemplary schematic is shown in FIG. 1. In a further embodiment, the intron sequence keeps the rAAV vector genome with a size between about 3 kilobases (kb) to about 6 kb, about 4.7 kb to about 6 kb, about 3 kb to about 5.5 kb, or about 4.7 kb to 5.5 kb. An example of a production plasmid which includes the TPP1 encoding sequence can be found in SEQ ID NO: 5. In another embodiment, the production plasmid is modified to optimized vector plasmid production efficiency. Such modifications include addition of other neutral sequences, or inclusion of a lambda stuffer sequence to modulate the level of supercoil of the vector plasmid. Such modifications are contemplated herein. In other embodiments, terminator and other sequences are included in the plasmid.

In certain embodiments, the rAAV expression cassette, the vector (such as rAAV vector), the virus (such as rAAV), and/or the production plasmid comprises AAV inverted terminal repeat sequences, a codon optimized nucleic acid sequence that encodes TPP1, and expression control sequences that direct expression of the encoded proteins in a host cell. In other embodiments, the rAAV expression cassette, the virus, the vector (such as rAAV vector), and/or the production plasmid further comprise one or more of an intron, a Kozak sequence, a polyA, post-transcriptional regulatory elements and others. In one embodiment, the post-transcriptional regulatory element is Woodchuck Hepatitis Virus (WHP) Posttranscriptional Regulatory Element (WPRE).

The expression cassettes, vectors and plasmids include other components that can be optimized for a specific species using techniques known in the art including, e.g., codon optimization, as described herein. The components of the cassettes, vectors, plasmids and viruses or other compositions described herein include a promoter sequence as part of the expression control sequences. In another embodiment, the promoter is cell-specific. The term “cell-specific” means that the particular promoter selected for the recombinant vector can direct expression of the optimized TPP1 coding sequence in a particular cell or tissue type. In one embodiment, the promoter is specific for expression of the transgene in ependyma, the epithelial lining of the brain ventricular system. In another embodiment, the promoter is specific for expression in a brain cell selected from neurons, astrocytes, oligoedendrocytes, and microglia. In one embodiment, the promoter is modified to add one or more restriction sites to facilitate cloning.

In another embodiment, the promoter is a ubiquitous or constitutive promoter. An example of a suitable promoter is a hybrid chicken 3-actin (CBA) promoter with cytomegalovirus (CMV) enhancer elements, such as the sequence shown in SEQ ID NO: 5 at nt 3396 to 4061. In another embodiment, the promoter is the CB7 promoter. Other suitable promoters include the human 3-actin promoter, the human elongation factor-1α promoter, the cytomegalovirus (CMV) promoter, the simian virus 40 promoter, and the herpes simplex virus thymidine kinase promoter. See, e.g., Damdindorj et al, (August 2014) A Comparative Analysis of Constitutive Promoters Located in Adeno-Associated Viral Vectors. PLoS ONE 9(8): e106472. Still other suitable promoters include viral promoters, constitutive promoters, regulatable promoters [see, e.g., WO 2011/126808 and WO 2013/04943]. Alternatively a promoter responsive to physiologic cues may be utilized in the expression cassette, rAAV genomes, vectors, plasmids and viruses described herein. In one embodiment, the promoter is of a small size, under 1000 bp, due to the size limitations of the AAV vector. In another embodiment, the promoter is under 400 bp. Other promoters may be selected by one of skill in the art.

In a further embodiment, the promoter is selected from SV40 promoter, the dihydrofolate reductase promoter, a phage lambda (PL) promoter, a herpes simplex viral (HSV) promoter, a tetracycline-controlled trans-activator-responsive promoter (tet) system, a long terminal repeat (LTR) promoter, such as a RSV LTR, MoMLV LTR, BIV LTR or an HIV LTR, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter. The promoter sequences thereof are known to one of skill in the art or available publically, such as in the literature or in databases, e.g., GenBank, PubMed, or the like.

In another embodiment, the promoter is an inducible promoter. The inducible promoter may be selected from known promoters including the rapamycin/rapalog promoter, the ecdysone promoter, the estrogen-responsive promoter, and the tetracycline-responsive promoter, or heterodimeric repressor switch. See, Sochor et al, An Autogenously Regulated Expression System for Gene Therapeutic Ocular Applications. Scientific Reports, 2015 Nov. 24; 5: 17105 and Daber R, Lewis M., A novel molecular switch. J Mol Biol. 2009 Aug. 28; 391(4):661-70, Epub 2009 Jun. 21 which are both incorporated herein by reference in their entirety.

In other embodiments, the expression cassette, vector, plasmid and virus described herein contain other appropriate transcription initiation, termination, enhancer sequences, efficient RNA processing signals such as splicing and polyadenylation (polyA) signals; TATA sequences; sequences that stabilize cytoplasmic mRNA; sequences that enhance translation efficiency (i.e., Kozak consensus sequence); introns; sequences that enhance protein stability; and when desired, sequences that enhance secretion of the encoded product. The expression cassette or vector may contain none, one or more of any of the elements described herein.

Examples of suitable polyA sequences include, e.g., a synthetic polyA or from bovine growth hormone (bGH), human growth hormone (hGH), SV40, rabbit β-globin (RGB), or modified RGB (mRGB). In a further embodiment, the poly A has a nucleic acid sequence from nt 33 to 159 of SEQ ID NO: 5.

Examples of suitable enhancers include, e.g., the CMV enhancer, the RSV enhancer, the alpha fetoprotein enhancer, the TTR minimal promoter/enhancer, LSP (TH-binding globulin promoter/alpha1-microglobulin/bikunin enhancer), an APB enhancer, ABPS enhancer, an alpha mic/bik enhancer, TTR enhancer, en34, ApoE amongst others.

In one embodiment, a Kozak sequence is included upstream of the TPP1 coding sequence to enhance translation from the correct initiation codon. In another embodiment, CBA exon 1 and intron are included in the expression cassette. In one embodiment, the TPP1 coding sequence is placed under the control of a hybrid chicken R actin (CBA) promoter. This promoter consists of the cytomegalovirus (CMV) immediate early enhancer, the proximal chicken R actin promoter, and CBA exon 1 flanked by intron 1 sequences.

In another embodiment, the intron is selected from CBA, human beta globin, IVS2, SV40, bGH, alpha-globulin, beta-globulin, collagen, ovalbumin, p53, or a fragment thereof.

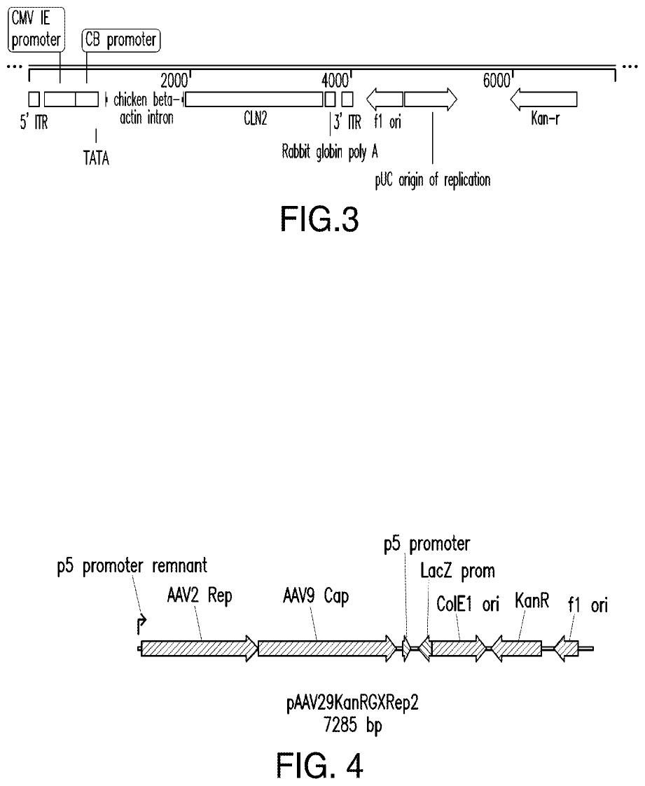

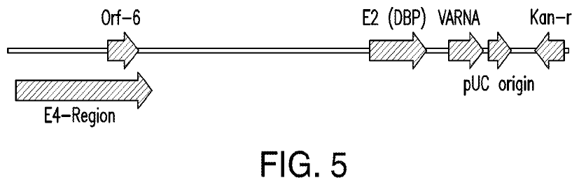

In one embodiment, the expression cassette, the vector, the plasmid and the virus contain a 5′ ITR, chicken beta-actin (CBA) promoter, CMV enhancer, CBA exon 1 and intron, human codon optimized CLN2 sequence, rabbit globin poly A and 3′ ITR. In a further embodiment, the expression cassette includes nt 1 to 4020 of SEQ ID NO: 8. In yet a further embodiment, the 5′ ITR has a nucleic acid sequence from nt 3199 to nt 3328 of SEQ ID NO: 5 and the 3′ITR has a nucleic acid sequence from nt 248 to nt 377 of SEQ ID NO: 5. In a further embodiment, the production plasmid has a sequence of SEQ ID NO: 5, also shown in FIGS. 1-5.

In particular embodiments, the rAAV is Construct III, which comprises, in 5′ to 3′ order: (1) a 5′ AAV2 ITR; (2) a CB7 promoter comprising (i) CMV immediate early enhancer, (ii) a chicken 3-actin promoter, and (iii) a chicken 3-actin intron; (3) an expression cassette comprising a human CLN2 transgene); (4) a rabbit β-globin poly A signal; and (5) a 3′AAV2 ITR. A schematic of Construct III is shown in FIG. 1.

In one aspect, a coding sequence is provided which encodes a functional TPP1 protein. By “functional hTPP1”, is meant a gene which encodes an TPP1 protein which provides at least about 50%, at least about 75%, at least about 80%, at least about 90%, or about the same, or greater than 100% of the biological activity level of the native TPP1 protein, or a natural variant or polymorph thereof which is not associated with disease.

In certain embodiments, the AAV9.CLN2 vector is produced. A number of suitable purification methods may be selected. Examples of suitable purification methods are described, e.g., International Patent Application No. PCT/US2016/065970, filed Dec. 9, 2016 and its priority documents, U.S. Patent Application Nos. 62/322,071, filed Apr. 13, 2016 and 62/226,357, filed Dec. 11, 2015 and entitled “Scalable Purification Method for AAV9”, which is incorporated by reference herein.

### 6.2 Pharmaceutical Compositions

In another aspect, also provided herein are pharmaceutical compositions comprising an rAAV described herein.

In certain embodiments, the pharmaceutical compositions provided herein comprises (a) a recombinant adeno-associated virus (rAAV), (b) sodium chloride, (c) magnesium chloride, (d) potassium chloride, (e) dextrose, (f) poloxamer 188, (g) sodium phosphate monobasic, and (h) sodium phosphate dibasic. In certain embodiments, the pharmaceutical composition further comprises calcium chloride. In certain embodiments, the rAAV is Construct III.

In certain embodiments, the rAAV in the pharmaceutical composition can be any rAAV that is known in the art. In certain embodiments, the rAAV in the pharmaceutical composition is any rAAV that is disclosed in the following patent applications, PCT/US2017/027650 (published as International Publication No.: WO 2017/181021), PCT/US2018/027568 (published as International Publication No.: WO 2018/191666), PCT/US2018/015910 (published as International Publication No.: WO 2018/144441), PCT/US2018/052855 (published as International Publication No.: WO 2019/067540), PCT/US2019/042205, PCT/US2019/043631, WO 2019079494 A1, WO 2019164854 A1, WO 2019079496 A2, US 20190211091A1, US 2019038777 A1, US 2018289839 A1, US 2019127455 A1, KR 20160010526 A, KR 20190086503 A, TW 201903146 A, WO 2019204514 A1, WO 2019204514 A1, WO 2019191114 A1, WO 2019169004 A1, WO 2019168961 A1, WO 2019164854 A1, WO 2019113224 A1, WO 2019108856 A1, WO 2019108857 A1, WO 2019060662 A1, WO 2019035066 A1, WO 2019036484 A1, WO 2019010335 A1, WO 2018232149 A1, WO 2018218359 A1, WO 2018209205 A1, WO 2018204626 A1, WO 2018200542 A1, WO 2018200419 A1, WO 2018191490 A1, WO 2018183293 A1, WO 2018160849 A1, WO 2018160582 A8, WO 2018160573 A1, WO 2018160585 A2, WO 2018152485 A1, WO 2018144709 A2, WO 2018126112 A1, WO 2018126116 A1, WO 2018059549 A1, WO 2018057916 A1, WO 2018022905 A2, WO 2018022511 A1, WO 2018009814 A1, WO 2017196814 A1, WO 2017184463 A1, WO 2017181068 A1, WO 2017180936 A1, WO 2017180854 A1, WO 2017180857 A1, WO 2017151884 A1, WO 2017151823 A1, WO 2017147180 A1, WO 2017136500 A1, WO 2017136533 A1, WO 2017120294 A1, WO 2017114497 A1, WO 2017106345 A1, WO 2017106354 A1, WO 2017106326 A1, WO 2017106244 A1, WO 2017106202 A2, WO 2017100676 A1, WO 2017100704 A1, WO 2017100674 A1, WO 2017100682 A1, WO 2017160360 A9, WO 2017087900 A1, WO 2017079656 A2, WO 2017062750 A1, WO 2017053732 A2, WO 2017040524 A1, WO 2017040528 A1, WO 2017024198 A1, WO 2017015102 A1, WO 2016196328 A1, WO 2016200543 A8, WO 2016179034 A2, WO 2016176191 A1, WO 2016176212 A1, WO 2016073556 A1, WO 2016019364 A1, WO 2015175639 A1, WO 2015164723 A1, WO 2015138870 A2, WO 2015138357 A2, WO 2015066627 A1, WO 2015009575 A1, WO 2015012924 A2, WO 2014151341 A1, WO 2014151265 A1, WO 2014124282 A1, WO 2014059068 A1, WO 2014052693 A2, WO 2014012025 A2, WO 2013173702 A2, WO 2013162748 A1, WO 2013142337 A1, WO 2014011210 A1, WO 2013049493 A1, WO 2012158757 A1, WO 2012145572 A1, WO 2012112832 A1, WO 2012071318 A2, WO 2011126808 A9, WO 2011112554 A1, WO 2011060233 A1, WO 2011041502 A1, WO 2011038187 A1, WO 2011038063 A1, WO 2010138675 A1, WO 2010127097 A1, WO 2010102140 A1, WO 2010056759 A1, WO 2010051367 A1, WO 2010062562 A1, WO 2010040135 A1, WO 2010011642 A2, WO 2010008782 A1, WO 2009134681 A2, WO 2009136977 A2, WO 2009105084 A2, WO 2009073104 A2, WO 2009073103 A2, WO 2008150459 A1, WO 2008140812 A2, WO 2008085486 A1, WO 2008079172 A2, WO 2008019131 A2, WO 2008013928 A2, WO 2007130455 A2, WO 2007127264 A2, WO 2008027084 A2, WO 2007106476 A2, WO 2007070705 A2, WO 2007024708 A2, WO 2007002285 A2, WO 2006110689 A2, WO 2006102072 A2, WO 2006039218 A2, WO 2006078279 A2, WO 2005118611 A2, WO 2005062957 A2, WO 2005033321 A2, WO 2005030292 A2, WO 2005027995 A2, WO 2005018431 A2, WO 2005001103 A2, WO 2004108922 A3, WO 2004094606 A2, WO 2004009769 A2, WO 03093460 A1, WO 03057171 A2, WO 03046124 A2, WO 03052051 A3, WO 03052052 A3, WO 03042397 A3, WO 03038062 A2, WO 03024502 A2, WO 03014367 A1, WO 03000851 A2, WO 02100317 A2, WO 02082904 A2, WO 0230410 A2, WO 0220718 A2, WO 0210410 A1, WO 0183692 A2, WO 0174163 A1, WO 0172329 A1, WO 0123001 A2, WO 0123597 A9, WO 0057837 A2, WO 0055342 A1, WO 0028061 A2, WO 9944645 A1, WO 9943360 A1, WO 9931982 A1, WO 9915677 A1, WO 9914354 A1, WO 9915685 A1, WO 9910013 A1, WO 9639530 A3, WO 9639416 A1, WO 9626286 A1, and WO 9613598 A2 (all publications, patents and patent applications referred to herein are incorporated by reference in their entirety for rAAVs that may be used).

In certain embodiments, the rAAV in the pharmaceutical composition may be selected from the group consisting of RGX-121 (REGENXBIO Inc.), RGX-111 (REGENXBIO Inc.), RGX-314 (REGENXBIO Inc.), Construct III (REGENXBIO Inc.), RGX-501 (REGENXBIO Inc.), Glybera® (alipogene tiparvovec) (uniQure), Voretigene neparvovec (SPK-RPE65) (Spark Therapeutics; MieraGTx UK II Ltd/Syne Qua Non Ltd/UCL), rAAV2-CBSB-hRPE65 (UPenn; NEI), rAAV2-hRPE65 (HMO), SPK-CHM (Spark Therapeutics), CNGA3-ACHM (AGTC), CNGB3-ACHM (AGTC), scAAV2-P1ND4 (NEI), XLRS gene therapy (Biogen/AGTC), BMN-270 (Biomarin), SB-525 (Sangamo), DTX101 (Dimension Therapeutics), SPK-9001 (SPK-FIX) (Spark Therapeutics/Pfizer), AMT-060 (uniQure/St. Jude's Hospital), SB-FIX (Sangamo), scAAV2/8-LP1-hFIXco (St. Jude's Hospital/UCL), ADVM-043 (Adverum), AVXS-101 (AveXis), rAAVrh74.MCK. micro-Dystrophin (NICHD), LGMD2D (NCH), rAAV1.CMV. huFollistatin344 (NCH), rAAVrh74.MHCK7.DYSF.DV (NCH), ART-102 (Arthrogen), Intracerebral gene therapy (INSERM), CERE-110 (Ceregene), CERE-120 (Ceregene/Sangamo), AAV-hAADC (NIH), AAV2CUhCLN2 (Weill Cornell University; Abeona Therapeutics), SAF-301 (Lysogene), DTX301 (Dimension Therapeutics), and TT-034 (Tacere Therapeutics) (see Naso et al. BioDrugs. 2017; 31(4): 317-334).

In certain embodiments, the rAAV in the pharmaceutical compositions may comprise components from one or more adeno-associated virus serotypes selected from the group consisting of AAV1, AAV2, AAV2tYF, AAV3, AAV4, AAV5, AAV6, AAV7, AAV8, AAV9, AAV10, AAV11, and AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16. In some embodiments, the rAAV in the pharmaceutical composition comprises a capsid protein of the AAV8 or AAV9 serotype. In preferred embodiments, the rAAV in the pharmaceutical composition comprises components from AAV9.

In certain embodiments, the pharmaceutical composition comprise multiple compounds. In certain embodiments, the compounds are in different hydrate forms, for example the hydrate forms selected from the group consisting of but not limited to anhydrous, monohydrate, dihydrate, 3-hydrate, 4-hydrate, 5-hydrate, 6-hydrate, 7-hydrate, 8-hydrate, 9-hydrate, and 10-hydrate forms.

In certain embodiments, the weight/volume concentration of a compound in the pharmaceutical composition may be expressed based on the compound in anhydrous form having a molar amount that is equivalent to the compound in a different hydrate form. In certain embodiments, the anhydrous form may not exist in nature.

In certain embodiments, the compound in certain hydrate form in the pharmaceutical composition may represent the same compound in a different hydrate form that has the equivalent molar amount.

In certain embodiments, the pharmaceutical composition comprises calcium chloride, for example calcium chloride in dihydrate form. In other embodiments, the pharmaceutical composition does not contain calcium chloride.

In certain embodiments, the pH of the pharmaceutical composition is about 7.4. In certain embodiments, the pH of the pharmaceutical composition is about 6.0 to 8.8. In certain embodiments, the pH of the pharmaceutical composition is about 6.0 to 9.0. In certain embodiments, the pH of the pharmaceutical composition is about 6.0. In certain embodiments, the pH of the pharmaceutical composition is about 6.1. In certain embodiments, the pH of the pharmaceutical composition is about 6.2. In certain embodiments, the pH of the pharmaceutical composition is about 6.3. In certain embodiments, the pH of the pharmaceutical composition is about 6.4. In certain embodiments, the pH of the pharmaceutical composition is about 6.5. In certain embodiments, the pH of the pharmaceutical composition is about 6.6. In certain embodiments, the pH of the pharmaceutical composition is about 6.7. In certain embodiments, the pH of the pharmaceutical composition is about 6.8. In certain embodiments, the pH of the pharmaceutical composition is about 6.9. In certain embodiments, the pH of the pharmaceutical composition is about 7.0. In certain embodiments, the pH of the pharmaceutical composition is about 7.1. In certain embodiments, the pH of the pharmaceutical composition is about 7.2. In certain embodiments, the pH of the pharmaceutical composition is about 7.3. In certain embodiments, the pH of the pharmaceutical composition is about 7.4. In certain embodiments, the pH of the pharmaceutical composition is about 7.5. In certain embodiments, the pH of the pharmaceutical composition is about 7.6. In certain embodiments, the pH of the pharmaceutical composition is about 7.7. In certain embodiments, the pH of the pharmaceutical composition is about 7.8. In certain embodiments, the pH of the pharmaceutical composition is about 7.9. In certain embodiments, the pH of the pharmaceutical composition is about 8.0. In certain embodiments, the pH of the pharmaceutical composition is about 8.1. In certain embodiments, the pH of the pharmaceutical composition is about 8.2. In certain embodiments, the pH of the pharmaceutical composition is about 8.3. In certain embodiments, the pH of the pharmaceutical composition is about 8.4. In certain embodiments, the pH of the pharmaceutical composition is about 8.5. In certain embodiments, the pH of the pharmaceutical composition is about 8.6. In certain embodiments, the pH of the pharmaceutical composition is about 8.7. In certain embodiments, the pH of the pharmaceutical composition is about 8.8. In certain embodiments, the pH of the pharmaceutical composition is about 8.9. In certain embodiments, the pH of the pharmaceutical composition is about 9.0.

In certain embodiments, the pH of the pharmaceutical composition is 7.4. In certain embodiments, the pH of the pharmaceutical composition is 6.0 to 8.8. In certain embodiments, the pH of the pharmaceutical composition is 6.0 to 9.0. In certain embodiments, the pH of the pharmaceutical composition is 6.0. In certain embodiments, the pH of the pharmaceutical composition is 6.1. In certain embodiments, the pH of the pharmaceutical composition is 6.2. In certain embodiments, the pH of the pharmaceutical composition is 6.3. In certain embodiments, the pH of the pharmaceutical composition is 6.4. In certain embodiments, the pH of the pharmaceutical composition is 6.5. In certain embodiments, the pH of the pharmaceutical composition is 6.6. In certain embodiments, the pH of the pharmaceutical composition is 6.7. In certain embodiments, the pH of the pharmaceutical composition is 6.8. In certain embodiments, the pH of the pharmaceutical composition is 6.9. In certain embodiments, the pH of the pharmaceutical composition is 7.0. In certain embodiments, the pH of the pharmaceutical composition is 7.1. In certain embodiments, the pH of the pharmaceutical composition is 7.2. In certain embodiments, the pH of the pharmaceutical composition is 7.3. In certain embodiments, the pH of the pharmaceutical composition is 7.4. In certain embodiments, the pH of the pharmaceutical composition is 7.5. In certain embodiments, the pH of the pharmaceutical composition is 7.6. In certain embodiments, the pH of the pharmaceutical composition is 7.7. In certain embodiments, the pH of the pharmaceutical composition is 7.8. In certain embodiments, the pH of the pharmaceutical composition is 7.9. In certain embodiments, the pH of the pharmaceutical composition is 8.0. In certain embodiments, the pH of the pharmaceutical composition is 8.1. In certain embodiments, the pH of the pharmaceutical composition is 8.2. In certain embodiments, the pH of the pharmaceutical composition is 8.3. In certain embodiments, the pH of the pharmaceutical composition is 8.4. In certain embodiments, the pH of the pharmaceutical composition is 8.5. In certain embodiments, the pH of the pharmaceutical composition is 8.6. In certain embodiments, the pH of the pharmaceutical composition is 8.7. In certain embodiments, the pH of the pharmaceutical composition is 8.8. In certain embodiments, the pH of the pharmaceutical composition is 8.9. In certain embodiments, the pH of the pharmaceutical composition is 9.0.

In certain embodiments, the pharmaceutical composition provided herein comprises a recombinant adeno-associated virus (rAAV) and one or more compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and one compound selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and two compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and three compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and four compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and five compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and six compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, the pharmaceutical composition provided herein comprises a recombinant adeno-associated virus (rAAV) and all seven compounds selected from the group consisting of sodium chloride, magnesium chloride, potassium chloride, dextrose, poloxamer 188, sodium phosphate monobasic, and sodium phosphate dibasic. In certain embodiments, the pharmaceutical composition further comprises calcium chloride.

In certain embodiments, provided herein is a pharmaceutical composition comprising:
(a) a recombinant adeno-associated virus (rAAV),
(b) sodium chloride,
(c) magnesium chloride,
(d) potassium chloride,
(e) dextrose,
(f) poloxamer 188,
(g) sodium phosphate monobasic, and
(h) sodium phosphate dibasic,
wherein said recombinant adeno-associated virus (rAAV) comprises an AAV capsid and a vector genome packaged therein, and wherein said vector genome comprising: (i) an AAV 5′ inverted terminal repeat (ITR) sequence; (ii) a promoter; (iii) a CLN2 coding sequence encoding a human TPP1; and (iv) an AAV 3′ ITR. In some embodiments, the rAAV is Construct III.

In certain embodiments, the pharmaceutical composition further comprising calcium chloride.

In certain embodiments, said sodium chloride, said magnesium chloride, said potassium chloride, said dextorse, said poloxamer 188, said sodium phosphate monobasic, said sodium phosphate dibasic, and said calcium chloride are each in anhydrous, monohydrate, dihydrate, 3-hydrate, 4-hydrate, 5-hydrate, 6-hydrate, 7-hydrate, 8-hydrate, 9-hydrate, or 10-hydrate form.

In certain embodiments, the pharmaceutical composition comprises
(a) said rAAV,
(b) sodium chloride at a concentration of about 8.77 g/L,
(c) magnesium chloride 6-hydrate, at a concentration of about 0.244 g/L,
(d) potassium chloride at a concentration of about 0.224 g/L,
(e) calcium chloride dihydrate at a concentration of about 0.206 g/L,
(f) dextorse anhydrous at a concentration of about 0.793 g/L,
(g) poloxamer 188 at a concentration of about 0.001% (volume/volume),
(h) sodium phosphate monobasic monohydrate at a concentration of about 0.0278 g/L, and
(i) sodium phosphate dibasic anhydrous at a concentration of about 0.114 g/L.

In certain embodiments, the vector genome concentration (VGC) of the pharmaceutical composition is about 1×10¹¹ GC/mL, about 3×10¹¹ GC/mL, about 6×10¹¹ GC/mL, about 1×10¹² GC/mL, about 3×10¹² GC/mL, about 6×10¹² GC/mL, about 1×10¹³ GC/mL, about 2×10¹³ GC/mL, about 3×10¹³ GC/mL, about 4×10¹³ GC/mL, about 5×10¹³ GC/mL, about 6×10¹³ GC/mL, about 7×10¹³ GC/mL, about 8×10¹³ GC/mL, about 9×10¹³ GC/mL, or about 1×10¹⁴ GC/mL, about 3×10¹⁴ GC/mL, about 6×10¹⁴ GC/mL, or about 1×10¹⁵ GC/mL. In certain embodiments, the vector genome concentration (VGC) of the pharmaceutical composition is 1×10¹¹ GC/mL, 3×10¹¹ GC/mL, 6×10¹¹ GC/mL, 1×10¹² GC/mL, 3×10¹² GC/mL, 6×10¹² GC/mL, 1×10¹³ GC/mL, 2×10¹³ GC/mL, about 3×10¹³ GC/mL, 4×10¹³ GC/mL, 5×10¹³ GC/mL, 6×10¹³ GC/mL, 7×10¹³ GC/mL, 8×10¹³ GC/mL, 9×10¹³ GC/mL, or 1×10¹⁴ GC/mL, 3×10¹⁴ GC/mL, 6×10¹⁴ GC/mL, or 1×10¹⁵ GC/mL.

In certain embodiments, the pH of the pharmaceutical composition is in a range from about 6.0 to about 9.0. In certain embodiments, the pH of the pharmaceutical composition is about 7.4.

In certain embodiments, the rAAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable to freeze/thaw cycles than the same recombinant rAAV in a reference pharmaceutical composition. In certain embodiments, the stability of the recombinant AAV is determined by an assay or assays disclosed in section 6.2.1.

In certain embodiments, the stability of said rAAV in the pharmaceutical composition is determined by
(a) the infectivity of rAAV,
(b) the levels of aggregation of rAAV, or
(c) the levels of free DNA released by the rAAV.

In certain embodiments, the pharmaceutical composition is a liquid composition. In certain embodiments, the pharmaceutical composition is a frozen composition. In certain embodiments, the pharmaceutical composition is a lyophilized composition or a reconstituted lyophilized composition.

In certain embodiments, the pharmaceutical composition has a property that is suitable for intracerebroventricular (ICV), intracisternal (IC), intrathecal-lumbar, intracranial, intravenous, intravascular, intraarterial, intramuscular, intraocular, intramuscular, subcutaneous, or intradermal administration.

In certain embodiments, the coding sequence of (iii) of the rAAV in the pharmaceutical composition is a codon optimized human CLN2, which is at least 70% identical to the native human coding sequence of SEQ ID NO: 2. In certain embodiments, the coding sequence of (iii) of the rAAV in the pharmaceutical composition is SEQ ID NO: 3.

In certain embodiments, the rAAV capsid of the rAAV in the pharmaceutical composition is an AAV9 or a variant thereof.

In certain embodiments, the promoter of the rAAV in the pharmaceutical composition is a chicken beta actin (CBA) promoter. In certain embodiments, the promoter of the rAAV in the pharmaceutical composition is a hybrid promoter comprising a CBA promoter sequence and cytomegalovirus enhancer elements.

In certain embodiments, the AAV 5′ ITR and/or AAV3′ ITR of the rAAV in the pharmaceutical composition is from AAV2.

In certain embodiments, the vector genome of the rAAV in the pharmaceutical composition further comprises a polyA. In certain embodiments, the polyA is a synthetic polyA or from bovine growth hormone (bGH), human growth hormone (hGH), SV40, rabbit β-globin (RGB), or modified RGB (mRGB).

In certain embodiments, the vector genome of the rAAV in the pharmaceutical composition further comprises an intron. In certain embodiments, the intron is from CBA, human beta globin, IVS2, SV40, bGH, alpha-globulin, beta-globulin, collagen, ovalbumin, or p53.

In certain embodiments, the vector genome of the rAAV in the pharmaceutical composition further comprises an enhancer. In certain embodiments, the enhancer is a CMV enhancer, an RSV enhancer, an APB enhancer, ABPS enhancer, an alpha mic/bik enhancer, TTR enhancer, en34, ApoE.

In certain embodiments, the vector genome of the rAAV in the pharmaceutical composition is about 3 kilobases to about 5.5 kilobases in size. In certain embodiments, the vector genome of the rAAV in the pharmaceutical composition is about 4 kilobases in size.

In certain embodiments, the rAAV in the pharmaceutical composition is manufactured using a method comprising growing in suspension culture a suspension cell line that is capable of producing the rAAV.

In yet another aspect, provide herein is a kit comprising one or more containers and instructions for use, wherein the one or more containers comprise the pharmaceutical composition provided herein.

In certain embodiments, the pharmaceutical composition comprises poloxamer 188 at a concentration of 0.001% (weight/volume, 0.01 g/L). In certain embodiments, the pharmaceutical composition comprises poloxamer 188 at a concentration of 0.0005% (weight/volume, 0.005 g/L) to 0.05% (weight/volume, 0.5 g/L. In certain embodiments, the pharmaceutical composition comprises poloxamer 188 at a concentration of 0.0001% (weight/volume, 0.001 g/L) to 0.01% (weight/volume, 0.1 g/L). In certain embodiments, the pharmaceutical composition comprises poloxamer 188 at a concentration of 0.0005% (weight/volume, 0.005 g/L) to 0.001% (weight/volume, 0.01 g/L). In certain embodiments, the pharmaceutical composition comprises poloxamer 188 at a concentration of 0.001% (weight/volume, 0.01 g/L) to 0.05% (weight/volume, 0.5 g/L). In certain embodiments, the pharmaceutical composition comprises poloxamer 188 at a concentration of 0.0005% (weight/volume, 0.005 g/L). In certain embodiments, the pharmaceutical composition comprises poloxamer 188 at a concentration of 0.0006% (weight/volume, 0.006 g/L). In certain embodiments, the pharmaceutical composition comprises poloxamer 188 at a concentration of 0.0007% (weight/volume, 0.007 g/L). In certain embodiments, the pharmaceutical composition comprises poloxamer 188 at a concentration of 0.0008% (weight/volume, 0.008 g/L). In certain embodiments, the pharmaceutical composition comprises poloxamer 188 at a concentration of 0.0009% (weight/volume, 0.009 g/L). In certain embodiments, the pharmaceutical composition comprises poloxamer 188 at a concentration of 0.001% (weight/volume, 0.01 g/L). In certain embodiments, the pharmaceutical composition comprises poloxamer 188 at a concentration of 0.002% (weight/volume, 0.02 g/L). In certain embodiments, the pharmaceutical composition comprises poloxamer 188 at a concentration of 0.003% (weight/volume, 0.03 g/L). In certain embodiments, the pharmaceutical composition comprises poloxamer 188 at a concentration of 0.004% (weight/volume, 0.04 g/L). In certain embodiments, the pharmaceutical composition comprises poloxamer 188 at a concentration of 0.005% (weight/volume, 0.05 g/L). In certain embodiments, the pharmaceutical composition comprises poloxamer 188 at a concentration of 0.01% (weight/volume, 0.1 g/L). In certain embodiments, the pharmaceutical composition comprises poloxamer 188 at a concentration of 0.05% (weight/volume, 0.5 g/L).

As used herein and unless otherwise specified, the term “about” means within plus or minus 10% of a given value or range.

The pharmaceutical compositions described herein are designed for delivery to subjects in need thereof by any suitable route or a combination of different routes.

In yet other aspects, these nucleic acid sequences, vectors, expression cassettes and rAAV viral vectors are useful in a pharmaceutical composition, which also comprises a pharmaceutically acceptable carrier, excipient, buffer, diluent, surfactant, preservative and/or adjuvant, etc. Such pharmaceutical compositions are used to express the optimized TPP1 in the host cells through delivery by such recombinantly engineered AAVs or artificial AAVs.

To prepare these pharmaceutical compositions containing the nucleic acid sequences, vectors, expression cassettes and rAAV viral vectors, the sequences or vectors or viral vector is preferably assessed for contamination by conventional methods and then formulated into a pharmaceutical composition suitable for administration to the patient. Such formulation involves the use of a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, surfactant, or excipient etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20.

In one exemplary specific embodiment, the composition of the carrier or excipient contains 180 mM NaCl, 10 mM NaPi, pH7.3 with 0.0001%-0.01% Pluronic F68 (PF68). The exact composition of the saline component of the buffer ranges from 160 mM to 180 mM NaCl. Optionally, a different pH buffer (potentially HEPES, sodium bicarbonate, TRIS) is used in place of the buffer specifically described. Still alternatively, a buffer containing 0.9% NaCl is useful.

In one embodiment, a method of generating a recombinant rAAV comprises obtaining a plasmid containing an AAV expression cassette as described above and culturing a packaging cell carrying the plasmid in the presence of sufficient viral sequences to permit packaging of the AAV viral genome into an infectious AAV envelope or capsid. Specific methods of rAAV vector generation are described above and may be employed in generating a rAAV vector that can deliver the codon optimized CLN2 in the expression cassettes and genomes described herein.

In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate contaminating host DNA from the production process. The DNase resistant particles are then subjected to heat treatment to release the genome from the capsid. The released genomes are then quantitated by real-time PCR using primer/probe sets targeting specific region of the viral genome (for example poly A signal). Another suitable method for determining genome copies are the quantitative-PCR (qPCR), particularly the optimized qPCR or digital droplet PCR [Lock Martin, et al, Human Gene Therapy Methods. April 2014, 25(2): 115-125. doi:10.1089/hgtb.2013.131, published online ahead of editing Dec. 13, 2013]. Alternatively, ViroCyt3100 can be used for particle quantitation, or flow cytometry. In another method, the effective dose of a recombinant adeno-associated virus carrying a nucleic acid sequence encoding the optimized TPP1 coding sequence is measured as described in S. K. McLaughlin et al, 1988 J. Virol., 62:1963, which is incorporated by reference in its entirety.

The replication-defective virus compositions can be formulated in dosage units to contain an amount of replication-defective virus that is in the range of about 1.0×10⁹ GC to about 9×10¹⁵ GC (to treat an average subject of 70 kg in body weight) including all integers or fractional amounts within the range, and preferably 1.0×10¹² GC to 2.7×10¹⁵ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 2.7×10¹⁵ GC per dose including all integers or fractional amounts within the range.

In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal or intracisternal delivery, a pH within this range may be desired; whereas for intravenous delivery, a pH of 6.8 to about 7.2 may be desired. In one embodiment, the pH is about 7.3. However, other pHs within the broadest ranges and these subranges may be selected for other route of delivery.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 (BASF), also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In one example, the formulation may contain, e.g., buffered saline solution comprising one or more of sodium chloride, sodium bicarbonate, dextrose, magnesium sulfate (e.g., magnesium sulfate·7H2O), potassium chloride, calcium chloride (e.g., calcium chloride·2H2O), dibasic sodium phosphate, and mixtures thereof, in water. Suitably, for intrathecal or intracisternal delivery, the osmolarity is within a range compatible with cerebrospinal fluid (e.g., about 275 to about 290); see, e.g., emedicine.medscape.com/article/2093316-overview. Optionally, for intrathecal or intracisternal delivery, a commercially available diluent may be used as a suspending agent, or in combination with another suspending agent and other optional excipients. See, e.g., Elliotts B® solution (Lukare Medical). In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

In another embodiment, the composition includes a carrier, solvent, stabilizer, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo.

In one embodiment, the AAV9.CB7.hCLN2 drug product proposed configuration is a 1 mL frozen solution of AAV9.CB7.hCLN2 vector in formulation buffer contained in a 2 mL vial. The proposed formulation buffer is 150 mM sodium chloride, 1.2 mM magnesium chloride, 3 mM potassium chloride, 1.4 mM calcium chloride, 1 mM sodium phosphate, 4.4 mM dextrose, and 0.001% poloxamer 188, pH 7.3. The proposed quantitative composition of AAV9.CB7.hCLN2 drug product is provided in Table 1 below.

TABLE 1
Proposed Quantitative Composition of AAV9.CB7.hCLN2
Solution for Injection, 1 mL/Vial

| | | | Amount | |
|---|---|---|---|---|
| Material | Function | Grade | (per vial) | |
| AAV9.CB7. | Active | GMP | >1 × 10¹³ | GC/mL |
| hCNLN2 | Substance | | | |
| Sodium | Stabiliser | USP/Ph. | 8.76 | mg/mL |
| Chloride | | Eur./JP/BP/FCC | | |
| Magnesium | Stabiliser | USP/Ph. | 0.11 | mg/mL |
| Chloride | | Eur./JP/BP/FCC | | |
| Potassium | Stabiliser | USP/Ph. | 0.22 | mg/mL |
| Chloride | | Eur./JP/BP/FCC | | |
| Calcium | Stabiliser | USP/Ph. | 0.16 | mg/mL |
| Chloride | | Eur./JP/BP/FCC | | |
| Sodium | Stabiliser | USP/Ph. | 0.16 | mg/mL |
| Phosphate | | Eur./JP/BP/FCC | | |
| Dextrose | Stabiliser | USP/Ph. | 0.79 | mg/mL |
| | | Eur./JP/BP/FCC | | |
| Poloxamer | Surfactant | GMP | 0.001 | mL |
| 188 | | | | |
| Water for | Solvent | USP/Ph. Eur. | q.s. to 1.0 | mL/vial |
| Injection | | | | |

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intrathecal delivery. In one embodiment, intrathecal delivery encompasses an injection into the spinal canal, e.g., the subarachnoid space. In one embodiment, the route of delivery is intracerebroventricular injection (ICV). In another embodiment, the route of delivery is intrathecal-lumbar (IT-L) delivery. In yet another embodiment, the route of delivery is intracisternal (IC) injection (i.e., intrathecal delivery via image-guided suboccipital puncture into the cisterna magna).

The viral vectors described herein may be used in preparing a medicament for delivering hTPP1 to a subject (e.g., a human patient) in need thereof, supplying functional TPP1 to a subject, and/or for treating CLN2 Disease. A course of treatment may optionally involve repeat administration of the same viral vector (e.g., an AAV9 vector) or a different viral vector (e.g., an AAV9 and an AAVrh10). Still other combinations may be selected using the viral vectors and non-viral delivery systems described herein.

The hTPP1 cDNA sequences described herein can be generated in vitro and synthetically, using techniques well known in the art. For example, the PCR-based accurate synthesis (PAS) of long DNA sequence method may be utilized, as described by Xiong et al, PCR-based accurate synthesis of long DNA sequences, Nature Protocols 1, 791-797 (2006). A method combining the dual asymmetrical PCR and overlap extension PCR methods is described by Young and Dong, Two-step total gene synthesis method, Nucleic Acids Res. 2004; 32(7): e59. See also, Gordeeva et al, J Microbiol Methods. Improved PCR-based gene synthesis method and its application to the Citrobacter freundii phytase gene codon modification. 2010 May; 81(2):147-52. Epub 2010 Mar. 10; see, also, the following patents on oligonucleotide synthesis and gene synthesis, Gene Seq. 2012 April; 6(1):10-21; U.S. Pat. Nos. 8,008,005; and 7,985,565. Each of these documents is incorporated herein by reference. In addition, kits and protocols for generating DNA via PCR are available commercially. These include the use of polymerases including, without limitation, Taq polymerase; OneTaq® (New England Biolabs); Q5@ High-Fidelity DNA Polymerase (New England Biolabs); and GoTaq® G2 Polymerase (Promega). DNA may also be generated from cells transfected with plasmids containing the hOTC sequences described herein. Kits and protocols are known and commercially available and include, without limitation, QIAGEN plasmid kits; Chargeswitch® Pro Filter Plasmid Kits (Invitrogen); and GenElute™ Plasmid Kits (Sigma Aldrich). Other techniques useful herein include sequence-specific isothermal amplification methods that eliminate the need for thermocycling. Instead of heat, these methods typically employ a strand-displacing DNA polymerase, like Bst DNA Polymerase, Large Fragment (New England Biolabs), to separate duplex DNA. DNA may also be generated from RNA molecules through amplification via the use of Reverse Transcriptases (RT), which are RNA-dependent DNA Polymerases. RTs polymerize a strand of DNA that is complimentary to the original RNA template and is referred to as cDNA. This cDNA can then be further amplified through PCR or isothermal methods as outlined above. Custom DNA can also be generated commercially from companies including, without limitation, GenScript; GENEWIZ®; GeneArt® (Life Technologies); and Integrated DNA Technologies.

The nucleic acid molecules, the expression cassette and/or vectors described herein may be delivered in a single composition or multiple compositions. Optionally, two or more different AAV may be delivered, or multiple viruses [see, e.g., WO 2011/126808 and WO 2013/049493]. In another embodiment, multiple viruses may contain different replication-defective viruses (e.g., AAV and adenovirus), alone or in combination with proteins.

### 6.2.1 Assays Related to Pharmaceutical Compositions

The skilled artisan may use the assays as described herein and/or techniques known in the art to study the composition and methods described herein, for example to test the formulations provided herein. More details on the assays are provided in Examples 3 and 4. Examples 3 and 4 also demonstrate in more detail how such assays can be used to test the formulations provided herein.

As described in Li et al., 2019 Cell & Gene Therapy Insights, 5(4):537-547 (incorporated by references herein in its entirety), exemplary assays include but are not limited the following: (1) Digital Droplet PCR (ddPCR) for Genome Copy Determinations; (2) Genome Content and % Full Capsid Analysis of AAV by Spectrophotometry; (3) Size Exclusion Chromatography to Determine DNA Distribution and Purity in Capsid; (4) Assessing Capsid Viral Protein Purity Using Capillary Electrophoresis; (5) In Vitro Potency Methods-Relative Infectivity as a Reliable Method for Quantifying Differences in the Infectivity of AAV Vectors in vitro; and (6) Analytical Ultracentrifugation (AUC) to Determine Capsid Empty/Full Ratios and Size Distributions.

Controlled freeze/thaw cycles can be run in the lyophilizer. Vials can be well-spaced on the shelves and 4 vials of buffer can be thermocoupled.

A temperature stress development stability study can be conducted at 1.0×10¹² GC/mL over 4 days at 37° C. to evaluate the relative stability of formulations provided herein.

Assays can be used to assess stability include but are not limited to in vitro relative potency (IVRP), vector genome concentration (VGC by ddPCR), free DNA by dye fluorescence, dynamic light scattering, appearance, and pH.

Long-term development stability studies can be carried out for 12 months to demonstrate maintenance of in-vitro relative potency and other quality at −80° C. (≤−60° C.) and −20° C. (−25° C. to −15° C.) in the formulations provided herein.

To relate the ddPCR GC titer to gene expression, an in vitro bioassay may be performed by transducing HEK293 cells and assaying the cell culture supernatant for anti-VEGF Fab protein levels. HEK293 cells are plated onto three poly-D-lysine-coated 96-well tissue culture plates overnight. The cells are then pre-infected with wild-type human Ad5 virus followed by transduction with three independently prepared serial dilutions of Construct II reference standard and test article, with each preparation plated onto separate plates at different positions. On the third day following transduction, the cell culture media is collected from the plates and measured for VEGF-binding Fab protein levels via ELISA. For the ELISA, 96-well ELISA plates coated with VEGF are blocked and then incubated with the collected cell culture media to capture anti-VEGF Fab produced by HEK293 cells. Fab-specific anti-human IgG antibody is used to detect the VEGF-captured Fab protein. After washing, horseradish peroxidase (HRP) substrate solution is added, allowed to develop, stopped with stop buffer, and the plates are read in a plate reader. The absorbance or OD of the HRP product is plotted versus log dilution, and the relative potency of each test article is calculated relative to the reference standard on the same plate fitted with a four-parameter logistic regression model after passing the parallelism similarity test, using the formula: EC50 reference+EC50 test article. The potency of the test article is reported as a percentage of the reference standard potency, calculated from the weighted average of the three plates.

To relate the ddPCR GC titer to functional gene expression, an in vitro bioassay may be performed by transducing HEK293 cells and assaying for transgene (e.g. enzyme) activity. HEK293 cells are plated onto three 96-well tissue culture plates overnight. The cells are then pre-infected with wild-type human adenovirus serotype 5 virus followed by transduction with three independently prepared serial dilutions of enzyme reference standard and test article, with each preparation plated onto separate plates at different positions. On the second day following transduction, the cells are lysed, treated with low pH to activate the enzyme, and assayed for enzyme activity using a peptide substrate that yields increased fluorescence signal upon cleavage by transgene (enzyme). The fluorescence or RFU is plotted versus log dilution, and the relative potency of each test article is calculated relative to the reference standard on the same plate fitted with a four-parameter logistic regression model after passing the parallelism similarity test, using the formula: EC50 reference+EC50 test article. The potency of the test article is reported as a percentage of the reference standard potency, calculated from the weighted average of the three plates.

Vector genome concentration GC can also be evaluated using ddPCR.

Free DNA can be determined by fluorescence of SYBR® Gold nucleic acid gel stain (‘SYBR Gold dye’) that is bound to DNA. The fluorescence can be measured using a microplate reader and quantitated with a DNA standard. The results in ng/μL can be reported.

Two approaches can be used to estimate the total DNA in order to convert the measured free DNA in ng/μL to a percentage of free DNA. In the first approach the GC/mL (OD) determined by UV-visible spectroscopy was used to estimate the total DNA in the sample, where M is the molecular weight of the DNA and 1×10⁶ is a unit conversion factor:

Total DNA (ng/μL) estimated=1×10⁶×GC/mL (OD)×M (g/mol)/6.02×10²³

In the second approach, the sample can be heated to 85° C. for 20 min with 0.05% poloxamer 188 and the actual DNA measured in the heated sample by the SYBR Gold dye assay can be used as the total. This therefore has the assumption that all the DNA was recovered and quantitated. For example, the determination of total DNA by the SYBR gold dye (relative to the UV reading) can be found to be 131% for the Construct II dPBS formulation and 152% for the Construct II modified dPBS with sucrose formulation (This variation in the conversion of ng/μL to percentage of free DNA can be captured as a range in the reported results). For trending, either the raw ng/μL can be used or the percentage determined by a consistent method can be used.

Size Exclusion Chromatography (SEC) can be performed using a Sepax SRT SEC-1000 Peek column (PN 215950P-4630, SN: 8A11982, LN: BT090, 5 μm 1000 A, 4.6×300 mm) on Waters Acquity Arc Equipment ID 0447 (C3PO), with a 25 mm pathlength flowcell. The mobile phase can be, for example, 20 mM sodium phosphate, 300 mM NaCl, 0.005% poloxamer 188, pH 6.5, with a flow rate of 0.35 mL/minute for 20 minutes, with the column at ambient temperature. Data collection can be performed with 2 point/second sampling rate and 1.2 nm resolution with 25 point mean smoothing at 214, 260, and 280 nm. The ideal target load can be 1.5E11 GC. The samples can be injected with 50 μL, about ⅓ of the ideal target or injected with 5 μL.

Dynamic light scattering (DLS) can be performed on a Wyatt DynaProIII using Corning 3540 384 well plates with a 30 μL sample volume. Ten acquisitions each for 10 s can be collected per replicate and there were three replicate measurements per sample. The solvent can be set according to the solvent used in the samples, for example ‘PBS’ for Construct II in dPBS and ‘4% sucrose’ for the Construct II in modified dPBS with sucrose samples. Results not meeting data quality criteria (baseline, SOS, noise, fit) can be ‘marked’ and excluded from the analysis. The low delay time cutoff can be changed from 1.4 μs to 10 μs for the modified dPBS with sucrose samples to eliminate the impact of the sucrose excipient peak at about 1 nm on causing artifactually low cumulants analysis diameter results.

Low temperature Differential Scanning Calorimetry (low-temp DSC) can be run using a TA Instruments DSC250. About 20 μL of sample can be loaded into a Tzero pan and crimped with a Tzero Hermetic lid. Samples can be equilibrated at 25° C. for 2 min, then cooled at 5° C./min to −60° C., equilibrated for 2 min, then heated at 5° C./min to 25° C. Heat flow data can be collected in conventional mode.

The pH of different formulation buffers was monitored with INLAB COOL PRO-ISM low temperature pH probe, which can detect pH down to −30° C. One milliliter buffer was placed in 15 mL Falcon tube and then the pH probe was submerged in the buffer. A piece of parafilm was used to seal the gap between Falcon tube and pH probe to avoid contamination and evaporation. The probe along with the Falcon tube was placed in −20 AD freezer. The pH and temperature of the buffer were recorded every 2.5 min for around 20 hour or until the pH versus temperature behavior achieved repeating pattern. The temperature change caused by the automatic defrosting process created a stress condition for buffer pH stability.

The osmometer uses the technique of freezing-point depression to measure osmolality. Calibration of the instrument can be performed using 50 mOsm/kg, 850 mOsm/kg, and 2000 mOsm/kg NIST traceable standards. The reference solution of 290 mOsm/kg can be used to determine the system suitability of the osmometer.

The density can be measured with Anton Paar DMA500 densitometer, using water as reference. The densitometer can be washed with water and then methanol, followed by air-drying between samples.

Viscosity can be measured using methods known in the art, for example methods provide in the United States Pharmacopeia (USP) published in 2019 and previous versions thereof (incorporated by reference herein in their entirety).

TCID₅₀ infectious titer assay as described in Frangois, et al. Molecular Therapy Methods & Clinical Development (2018) Vol. 10, pp. 223-236 (incorporated by reference herein in its entirety) can be used. Relative infectivity assay as described in Provisional Application 62/745,859 filed Oct. 15, 2018) can be used.

Exemplary methods are described in Croyle et al., 2001, Gene Ther. 8(17):1281-90 (incorporated by reference in its entirety herein).

### 6.3 Method of Treating CLN2 Disease

In another aspect, provided herein are methods for treating CLN2 Disease in a subject, comprising administering to the subject an rAAV or a pharmaceutical composition described herein.

As used herein, the terms “Late Infantile Neuronal Ceroid Lipofuscinosis Type 2 (CLN2)” or “CLN2 disease” or “CLN2 Batten disease” are used interchangeably and refer to a disease caused by a defect in the TPP1 gene. CL.N2 disease is one ofa group of disorders known as neuronal ceroid lipofascinoses (NCILs which may also be collectively referred to as CLN2 Disease.

Neuronal ceroid-lipofuscinoses (NCLs), are a group of inherited, neurodegenerative, lysosomal storage disorders characterized by progressive intellectual and motor deterioration, seizures, and early death. Visual loss is a feature of most forms. Clinical phenotypes have been characterized traditionally according to the age of onset and order of appearance of clinical features into infantile, late-infantile, juvenile, adult, and Northern epilepsy (also known as progressive epilepsy with mental retardation [EPMR]). There is however genetic and allelic heterogeneity; a proposed new nomenclature and classification system has been developed to take into account both the responsible gene and the age at disease onset; for example, CLN2 disease, classic late infantile. The first symptoms typically appear between age two and four years, usually starting with epilepsy, followed by regression of developmental milestones, myoclonic ataxia, and pyramidal signs. Visual impairment typically appears at age four to six years and rapidly progresses to light/dark awareness only. Life expectancy ranges from age six years to early teenage.

In certain embodiments, the subject has a documented diagnosis of CLN2 disease due to TPP1 deficiency. The diagnosis may be confirmed by biochemical, molecular, or genetic methods.

In certain embodiments of this invention, a subject has neuronal ceroid lipofuscinosis (NCL), for which the components, compositions and methods of this invention are designed to treat.

In some embodiments, a method for treating CLN2 Disease caused by a defect in the CLN2 gene comprises delivering to a subject in need thereof a vector (such as rAAV) which encodes TPP1, as described herein. In one embodiment, a method of treating a subject having CLN2 Disease with a rAAV described herein (e.g., Construct III) is provided. Also provided herein are methods of treating CLN2 Disease comprising administering to a subject in need thereof the rAAV described herein via more than one route. In certain embodiments, said rAAV is administered in a therapeutically effective amount.

As used herein, the term “subject” as used herein means a mammalian animal, including a human, a veterinary or farm animal, a domestic animal or pet, and animals normally used for clinical research. In one embodiment, the subject of these methods and compositions is a human. Still other suitable subjects include, without limitation, murine, rat, canine, feline, porcine, bovine, ovine, non-human primate and others. As used herein, the term “subject” is used interchangeably with “patient”. In certain embodiments, said subject is human. In certain embodiments, the subject is between 4 months and 6 years of age.

As used herein, the term “treatment” or “treating” is defined encompassing administering to a subject one or more compounds or compositions described herein for the purposes of amelioration of one or more symptoms of CLN2 Disease. “Treatment” can thus include one or more of reducing onset or progression of neuronal ceroid lipofuscinosis (NCL), preventing disease, reducing the severity of the disease symptoms, or retarding their progression, including the progression of blindness, removing the disease symptoms, delaying onset of disease or monitoring progression of disease or efficacy of therapy in a given subject.

### 6.3.1 Dosage and Route of Administration

The pharmaceutical compositions described herein (e.g., described in section 6.2) or the rAAV described herein (e.g., described in section 6.1) may be administered to a subject in need thereof by any suitable route or a combination of different routes. In some embodiments, direct delivery to the brain (optionally via intrathecal, intracisternal, ICV or IT-L injection), or delivery via systemic routes is employed, e.g., intravascular, intraarterial, intraocular, intravenous, intramuscular, subcutaneous, intradermal, and other parental routes of administration. The Routes of administration may be combined, if desired. In some embodiments, the administration is repeated periodically.

In one embodiment, a method of treatment described herein comprises delivering the rAAV or the composition by intrathecal injection. In another embodiment, ICV injection to the subject is employed. In another embodiment, intrathecal-lumbar (IT-L) injection to the subject is employed. In one embodiment, the method involves delivering the composition via intracisternal (IC) injection (i.e., intrathecal delivery via image-guided suboccipital puncture into the cisterna magna). As used herein, the term intrathecal may, in some embodiments, refer to intracisternal injection. In still another method, intravascular injections may be employed. In another embodiment, intramuscular injection is employed.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna.

As used herein, the terms “intracisternal delivery” or “intracisternal administration” refer to a route of administration for drugs directly into the cerebrospinal fluid of the cisterna magna cerebello medularis, more specifically via a suboccipital puncture or by direct injection into the cisterna magna or via permanently positioned tube. A device which is useful for delivering the compositions described herein into cerebrospinal fluid is described in PCT/US2017/16133, which is incorporated herein by reference.

Provided herein in one aspect, is a method of treating CLN2 Disease in a subject comprising administering to a subject in need thereof an rAAV or a composition described herein via a first route and a second route, and said first route and said second route are into the central nervous system (CNS), and said first route is into the brain region and said second route is into the spinal cord region, and said rAAV comprises an AAV capsid and a vector genome packaged therein, and wherein said vector genome comprising: (a) an AAV 5′ inverted terminal repeat (ITR) sequence; (b) a promoter; (c) a CLN2 coding sequence encoding a human TPP1; and (d) an AAV 3′ ITR. In some embodiments, the rAAV is Construct III.

In certain embodiments, the brain region may be the intrathecal space covering the brain. In certain embodiments, the brain region may be the cerebral ventricles. In certain embodiments, the brain region may be the cisterna magna. In certain embodiments, delivery into the brain region may be delivering into the cerebrospinal fluid (CSF).

In certain embodiments, the spinal cord region may be the intrathecal space around the spinal cord. In certain embodiments, the spinal cord region may be the spinal canal. In certain embodiments, the spinal cord region may be the subarachnoid space. In certain embodiments, delivery into the spinal cord region may be delivering into the cerebrospinal fluid (CSF).

In certain embodiments, the first route is intracerebroventricular (ICV) or intracisternal (IC). In other embodiments, the first route is an administration route into the brain region that is other than intracerebroventricular (ICV) or intracisternal (IC).

In certain embodiments, the second route is intrathecal-lumbar (IT-L). In other embodiments, the first route is an administration route into the spinal cord region that is other than intrathecal-lumbar (IT-L).

In certain embodiments, the method further comprises administering to the subject the rAAV or the composition via a third route, wherein the third route is selected from the group consisting of intracerebroventricular (ICV), intracisternal (IC), intrathecal-lumbar, intracranial, intravenous, intravascular, intraarterial, intramuscular, intraocular, subcutaneous, and intradermal. In certain embodiments, the third route delivers the rAAV to the liver. In a specific embodiment, said third route is intravenous.

In certain embodiments, the method of treating CLN2 Disease in a subject comprises co-administering to a subject in need thereof an rAAV or a composition provided herein via intracerebroventricular (ICV) and intrathecal-lumbar (IT-L) routes. In certain embodiments, the method of treating CLN2 Disease in a subject comprises co-administering to a subject in need thereof an rAAV or a composition provided herein via intracisternal (IC) and intrathecal-lumbar (IT-L) routes. In certain embodiments, the method of treating CLN2 Disease in a subject comprises co-administering to a subject in need thereof an rAAV or a composition provided herein via intracerebroventricular (ICV), intrathecal-lumbar (IT-L), and intravenous routes. In certain embodiments, the method of treating CLN2 Disease in a subject comprises co-administering to a subject in need thereof an rAAV or a composition provided herein via intracisternal (IC), intrathecal-lumbar (IT-L), and intravenous routes.

In another aspect, provided is a method of treating CLN2 Disease in a subject comprising administering to a subject in need thereof an rAAV or a composition provided herein via a first route and a second route, wherein the first route is into the central nervous system (CNS), and the second route delivers the rAAV outside of the CNS, and the rAAV comprises an AAV capsid and a vector genome packaged therein, and wherein said vector genome comprising: (a) an AAV 5′ inverted terminal repeat (ITR) sequence; (b) a promoter; (c) a CLN2 coding sequence encoding a human TPP1; and (d) an AAV 3′ ITR. In certain embodiments, the first route is intrathecal-lumbar (IT-L), intracerebroventricular (ICV) or intracisternal (IC). In certain embodiments, the second route is selected from the group consisting of intravenous, intravascular, intraarterial, intramuscular, intraocular, subcutaneous, and intradermal. In a specific embodiment, the second route is intravenous.

In another aspect, provided is a method of treating CLN2 Disease in a subject comprising administering to a subject in need thereof an rAAV or a composition provided herein via a first route and a second route, wherein the first route is into the central nervous system (CNS), and the second route delivers the rAAV to the liver, and the rAAV or composition provided herein comprises an AAV capsid and a vector genome packaged therein, and wherein said vector genome comprising: (a) an AAV 5′ inverted terminal repeat (ITR) sequence; (b) a promoter; (c) a CLN2 coding sequence encoding a human TPP1; and (d) an AAV 3′ ITR. In certain embodiments, the first route is intrathecal-lumbar (IT-L), intracerebroventricular (ICV) or intracisternal (IC). In other embodiments, the first route is an administration route into the CNS that is other than intrathecal-lumbar (IT-L), intracerebroventricular (ICV) or intracisternal (IC). In certain embodiments, the second route is selected from the group consisting of intravenous, intravascular, intraarterial, intramuscular, intraocular, subcutaneous, and intradermal. In a specific embodiment, the second route is intravenous. In other embodiments, the second route is an administration route delivering the rAAV to the liver that is other than intravenous, intravascular, intraarterial, intramuscular, intraocular, subcutaneous, and intradermal.

In certain embodiments, the method of treating CLN2 Disease in a subject comprises co-administering to a subject in need thereof an rAAV or a composition provided herein into the CNS and intravenous route. In certain embodiments, the method of treating CLN2 Disease in a subject comprises co-administering to a subject in need thereof an rAAV or a composition provided herein via intrathecal and intravenous routes. In certain embodiments, the method of treating CLN2 Disease in a subject comprises co-administering to a subject in need thereof an rAAV or a composition provided herein via intrathecal-lumbar (IT-L) and intravenous routes. In certain embodiments, the method of treating CLN2 Disease in a subject comprises co-administering to a subject in need thereof an rAAV or a composition provided herein via intracerebroventricular (ICV) and intravenous routes. In certain embodiments, the method of treating CLN2 Disease in a subject comprises co-administering to a subject in need thereof an rAAV or a composition provided herein via intracisternal (IC) and intravenous routes. In certain embodiments, the method of treating CLN2 Disease in a subject comprises co-administering to a subject in need thereof an rAAV or a composition provided herein via a route into the CNS, which is other than intrathecal-lumbar (IT-L), intracerebroventricular (ICV) or intracisternal (IC), and intravenous routes.

In certain embodiments, the method of treating CLN2 Disease in a subject comprises co-administering to a subject in need thereof an rAAV or a composition provided herein via a route into the CNS and intravascular route. In certain embodiments, the method of treating CLN2 Disease in a subject comprises co-administering to a subject in need thereof an rAAV or a composition provided herein via intrathecal and intravascular routes. In certain embodiments, the method of treating CLN2 Disease in a subject comprises co-administering to a subject in need thereof an rAAV or a composition provided herein via intrathecal-lumbar (IT-L) and intravascular routes. In certain embodiments, the method of treating CLN2 Disease in a subject comprises co-administering to a subject in need thereof an rAAV or a composition provided herein via intracerebroventricular (ICV) and intravascular routes. In certain embodiments, the method of treating CLN2 Disease in a subject comprises co-administering to a subject in need thereof an rAAV or a composition provided herein via intracisternal (IC) and intravascular routes. In certain embodiments, the method of treating CLN2 Disease in a subject comprises co-administering to a subject in need thereof an rAAV or a composition provided herein via a route into the CNS, which is other than intrathecal-lumbar (IT-L), intracerebroventricular (ICV) or intracisternal (IC), and intravascular routes.

In certain embodiments, the method of treating CLN2 Disease in a subject comprises co-administering to a subject in need thereof an rAAV or a composition provided herein via a route into the CNS and intraarterial route. In certain embodiments, the method of treating CLN2 Disease in a subject comprises co-administering to a subject in need thereof an rAAV or a composition provided herein via intrathecal and intraarterial routes. In certain embodiments, the method of treating CLN2 Disease in a subject comprises co-administering to a subject in need thereof an rAAV or a composition provided herein via intrathecal-lumbar (IT-L) and intraarterial routes. In certain embodiments, the method of treating CLN2 Disease in a subject comprises co-administering to a subject in need thereof an rAAV or a composition provided herein via intracerebroventricular (ICV) and intraarterial routes. In certain embodiments, the method of treating CLN2 Disease in a subject comprises co-administering to a subject in need thereof an rAAV or a composition provided herein via intracisternal (IC) and intraarterial routes. In certain embodiments, the method of treating CLN2 Disease in a subject comprises co-administering to a subject in need thereof an rAAV or a composition provided herein via a route into the CNS, which is other than intrathecal-lumbar (IT-L), intracerebroventricular (ICV) or intracisternal (IC), and intraarterial routes.

In certain embodiments, the method of treating CLN2 Disease in a subject comprises co-administering to a subject in need thereof an rAAV or a composition provided herein via a route into the CNS and intramuscular route. In certain embodiments, the method of treating CLN2 Disease in a subject comprises co-administering to a subject in need thereof an rAAV or a composition provided herein via intrathecal and intramuscular routes. In certain embodiments, the method of treating CLN2 Disease in a subject comprises co-administering to a subject in need thereof an rAAV or a composition provided herein via intrathecal-lumbar (IT-L) and intramuscular routes. In certain embodiments, the method of treating CLN2 Disease in a subject comprises co-administering to a subject in need thereof an rAAV or a composition provided herein via intracerebroventricular (ICV) and intramuscular routes. In certain embodiments, the method of treating CLN2 Disease in a subject comprises co-administering to a subject in need thereof an rAAV or a composition provided herein via intracisternal (IC) and intramuscular routes. In certain embodiments, the method of treating CLN2 Disease in a subject comprises co-administering to a subject in need thereof an rAAV or a composition provided herein via a route into the CNS, which is other than intrathecal-lumbar (IT-L), intracerebroventricular (ICV) or intracisternal (IC), and intramuscular routes.

In certain embodiments, the method of treating CLN2 Disease in a subject comprises co-administering to a subject in need thereof an rAAV or a composition provided herein via a route into the CNS and intraocular route. In certain embodiments, the method of treating CLN2 Disease in a subject comprises co-administering to a subject in need thereof an rAAV or a composition provided herein via intrathecal and intraocular routes. In certain embodiments, the method of treating CLN2 Disease in a subject comprises co-administering to a subject in need thereof an rAAV or a composition provided herein via intrathecal-lumbar (IT-L) and intraocular routes. In certain embodiments, the method of treating CLN2 Disease in a subject comprises co-administering to a subject in need thereof an rAAV or a composition provided herein via intracerebroventricular (ICV) and intraocular routes. In certain embodiments, the method of treating CLN2 Disease in a subject comprises co-administering to a subject in need thereof an rAAV or a composition provided herein via intracisternal (IC) and intraocular routes. In certain embodiments, the method of treating CLN2 Disease in a subject comprises co-administering to a subject in need thereof an rAAV or a composition provided herein via a route into the CNS, which is other than intrathecal-lumbar (IT-L), intracerebroventricular (ICV) or intracisternal (IC), and intraocular routes.

In certain embodiments, the method of treating CLN2 Disease in a subject comprises co-administering to a subject in need thereof an rAAV or a composition provided herein via a route into the CNS and subcutaneous routes. In certain embodiments, the method of treating CLN2 Disease in a subject comprises co-administering to a subject in need thereof an rAAV or a composition provided herein via intrathecal and subcutaneous routes. In certain embodiments, the method of treating CLN2 Disease in a subject comprises co-administering to a subject in need thereof an rAAV or a composition provided herein via intrathecal-lumbar (IT-L) and subcutaneous routes. In certain embodiments, the method of treating CLN2 Disease in a subject comprises co-administering to a subject in need thereof an rAAV or a composition provided herein via intracerebroventricular (ICV) and subcutaneous routes. In certain embodiments, the method of treating CLN2 Disease in a subject comprises co-administering to a subject in need thereof an rAAV or a composition provided herein via intracisternal (IC) and subcutaneous routes. In certain embodiments, the method of treating CLN2 Disease in a subject comprises co-administering to a subject in need thereof an rAAV or a composition provided herein via a route into the CNS, which is other than intrathecal-lumbar (IT-L), intracerebroventricular (ICV) or intracisternal (IC), and subcutaneous routes.

In certain embodiments, the method of treating CLN2 Disease in a subject comprises co-administering to a subject in need thereof an rAAV or a composition provided herein via a route into the CNS and intradermal route. In certain embodiments, the method of treating CLN2 Disease in a subject comprises co-administering to a subject in need thereof an rAAV or a composition provided herein via intrathecal and intradermal routes. In certain embodiments, the method of treating CLN2 Disease in a subject comprises co-administering to a subject in need thereof an rAAV or a composition provided herein via intrathecal-lumbar (IT-L) and intradermal routes. In certain embodiments, the method of treating CLN2 Disease in a subject comprises co-administering to a subject in need thereof an rAAV or a composition provided herein via intracerebroventricular (ICV) and intradermal routes. In certain embodiments, the method of treating CLN2 Disease in a subject comprises co-administering to a subject in need thereof an rAAV or a composition provided herein via intracisternal (IC) and intradermal routes. In certain embodiments, the method of treating CLN2 Disease in a subject comprises co-administering to a subject in need thereof said rAAV via a route into the CNS, which is other than intrathecal-lumbar (IT-L), intracerebroventricular (ICV) or intracisternal (IC), and intradermal routes.

In certain embodiments, methods of treating CLN2 Disease provided herein may comprise administering an rAAV or a composition provided herein via said first route simultaneously with administering the rAAV or composition via said second route.

In certain embodiments, methods of treating CLN2 Disease provided herein may comprise administering an rAAV or a composition provided herein via said first route prior to administering the rAAV or a composition via said second route. In certain embodiments, methods of treating CLN2 Disease provided herein may comprise administering an rAAV or a composition provided herein via said first route after administering the rAAV or composition via said second route.

In certain embodiments, the interval between administration an rAAV or a composition provided herein via said first route and administering the rAAV or composition via said second route may be about 0.5 hour, 1 hour, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, or more.

In certain embodiments, the interval between administration an rAAV or a composition provided herein via said first route and administering the rAAV or composition via said second route may be 0.5 hour, 1 hour, 2 hours, 3 hours, 4 hours, 5 hours, 6 hours, 7 hours, 8 hours, 9 hours, 10 hours, 11 hours, 12 hours, about 1 day, 2 days, 3 days, 4 days, 5 days, 6 days, 1 week, 8 days, 9 days, 10 days, 11 days, 12 days, 13 days, 2 weeks, 3 weeks, 4 weeks, 5 weeks, 6 weeks, 7 weeks, 8 weeks, 9 weeks, 10 weeks, 11 weeks, 12 weeks, 1 month, 2 months, 3 months, 4 months, 5 months, 6 months, or more.

The amount of genome copies (“GC”) of an rAAV described herein that is administered to a subject may be determined based on the subject's brain mass. It is known in the art that the mass of the average human brain is about 1,300 g to about 1,400 g. It is also contemplated that the compositions here are useful in children, which have a range of brain mass from about 1000 g to about 1300 g. The brain mass of a subject may be derived from the subject's estimated brain volume as determined, for example, by magnetic resonance imaging (MRI).

All dosages may be measured by any known method, including as measured by qPCR or digital droplet PCR (ddPCR) as described in, e.g., M. Lock et al, Hum Gene Ther Methods. 2014 April; 25(2):115-25. doi: 10.1089/hgtb.2013.131, which is incorporated herein by reference.

In certain embodiments, a method of treating CLN2 Disease described herein comprises administering to the subject about 1×10⁹, about 2×10⁹, about 3×10⁹, about 4×10⁹, about 5×10⁹, about 6×10⁹, about 7×10⁹, about 8×10⁹, about 9×10⁹, about 1×10¹⁰, about 2×10¹⁰, about 3×10¹⁰, about 4×10¹⁰, about 5×10¹⁰, about 6×10¹⁰, about 7×10¹⁰, about 8×10¹⁰, about 9×10¹⁰, about 1×10¹¹, about 2×10¹¹, about 3×10¹¹, about 4×10¹¹, about 5×10¹¹, about 6×10¹¹, about 7×10¹¹, about 8×10¹¹, about 9×10¹¹, about 1×10¹², about 2×10¹², about 3×10¹², about 4×10¹², about 5×10¹², about 6×10¹², about 7×10¹², about 8×10¹², about 9×10¹²′ about 1×10¹³, about 2×10¹³, about 3×10¹³, about 4×10¹³, about 5×10¹³, about 6×10¹³, about 7×10¹³, about 8×10¹³, about 9×10¹³, about 1×10¹⁴, about 2×10¹⁴, about 3×10¹⁴, about 4×10¹⁴, about 5×10¹⁴, about 6×10¹⁴, about 7×10¹⁴, about 8×10¹⁴, about 9×10¹⁴, or about 1×10¹⁵ GC/g brain mass of an rAAV provided herein. In some embodiments, a method of treating CLN2 Disease described herein comprises administering to the subject about 1.25×10¹¹ GC/g brain mass or 4.5×10¹¹ GC/g brain mass of an rAAV provided herein.

In certain embodiments, a method of treating CLN2 Disease described herein comprises administering to the subject about 1×10⁹ to about 2×10⁹, about 2×10⁹ to about 3×10⁹, about 3×10⁹ to about 4×10⁹, about 4×10⁹ to about 5×10⁹, about 5×10⁹ to about 6×10⁹, about 6×10⁹ to about 7×10⁹, about 7×10⁹ to about 8×10⁹, about 8×10⁹ to about 9×10⁹, about 9×10⁹ to about 1×10¹⁰, about 1×10¹⁰ to about 2×10¹⁰, about 2×10¹⁰ to about 3×10¹⁰, about 3×10¹⁰ to about 4×10¹⁰, about 4×10¹⁰ to about 5×10¹⁰, about 5×10¹⁰ to about 6×10¹⁰, about 6×10¹⁰ to about 7×10¹⁰, about 7×10¹⁰ to about 8×10¹⁰, about 8×10¹⁰ to about 9×10¹⁰, about 9×10¹⁰ to about 1×10¹¹, about 1×10¹¹ to about 2×10¹¹, about 2×10¹¹ to about 3×10¹¹, about 3×10¹¹ to about 4×10¹¹, about 4×10¹¹ to about 5×10¹¹, about 5×10¹¹ to about 6×10¹¹, about 6×10¹¹ to about 7×10¹¹, about 7×10¹¹ to about 8×10¹¹, about 8×10¹¹ to about 9×10¹¹, about 9×10¹¹ to about 1×10¹², about 1×10¹² to about 2×10¹², about 2×10¹² to about 3×10¹², about 3×10¹² to about 4×10¹², about 4×10¹² to about 5×10¹², about 5×10¹² to about 6×10¹², about 6×10¹² to about 7×10¹², about 7×10¹² to about 8×10¹², about 8×10¹² to about 9×10¹², about 9×10¹² to about 1×10¹³, about 1×10¹³ to about 2×10¹³, about 12×10¹³ to about 3×10¹³, about 3×10¹³ to about 4×10¹³, about 4×10¹³ to about 5×10¹³, about 5×10¹³ to about 6×10¹³, about 6×10¹³ to about 7×10¹³, about 7×10¹³ to about 8×10¹³, about 8×10¹³ to about 9×10¹³, about 9×10¹³ to about 1×10¹⁴, about 1×10¹⁴ to about 2×10¹⁴, about 2×10¹⁴ to about 3×10¹⁴, about 3×10¹⁴ to about 4×10¹⁴, about 4×10¹⁴ to about 5×10¹⁴, about 5×10¹⁴ to about 6×10¹⁴, about 6×10¹⁴ to about 7×10¹⁴, about 7×10¹⁴ to about 8×10¹⁴, about 8×10¹⁴ to about 9×10¹⁴, or about 9×10¹⁴ to about 1×10¹⁵ GC/g brain mass of an rAAV provided herein.

Suitable volumes for delivery of these doses and concentrations may be determined by one of skill in the art. For example, volumes of about 1 μL to 150 mL may be selected, with the higher volumes being selected for adults. In one embodiment, the volume is about 10 mL or less. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. In still other embodiments, a patient may receive an intracisternal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

### 6.3.2 Methods of Assessing Efficacy

The efficacy of a method of treating CLN2 Disease described herein may be determined by any method known in the art, e.g., the methods described in this section. The efficacy of a method of treating CLN2 Disease described herein may be determined at any point after treatment, e.g., at about 3 months, about 6 months, about 9 months, about 12 months, about 15 months, about 18 months, about 21 months, about 24 months, about 27 months, about 30 months, about 33 months, about 36 months, about 39 months, about 42 months, about 45 months, about 48 months, about 51 months, about 54 months, about 57 months, about 60 months, about 63 months, about 66 months, about 69 months, or at about 72 months after treating. In some embodiments, the efficacy of a method of treating CLN2 Disease provided herein is assessed repeatedly after treatment, e.g., once a months, every 2 months, every 3 months, every 6 months, once a year, every 2 years, every 3 years, every 4 years, every 5 years, every 10 years or every 5 years.

In some embodiments, the efficacy of a method treating CLN2 Disease described herein is assessed using CLN2 clinical rating scales (CLN2 CRS). Two related CLN2 CRSs have been developed specific to CLN2 disease to assess individuals' change in Motor Function, Language, Seizure, and Vision over time. The original 12-point Hamburg scale (Steinfield et al., Am J Med Genet. 2002; 112:347-54) includes all 4 domains, and the 2018 update to the scale (Wyrwich et al., J Inborn Errors Metab Screen. 2018; 6:1-7) revised the scale wording for motor and language to form a combined 0- to 6-point Motor-Language domain (CLN2 CRS M and L). The highest score for each domain is 3 points, corresponding to normal or baseline ability, whereas a score of 0 indicates no ability in that domain. These scales have been previously used in clinical studies, such as the 6 point Motor-Language scale used to assess Brineura's clinical efficacy (Wyrwich, 2018). The CLN2 CRS Motor Language and Motor domains may be used individually and/or with a combined score.

The Expanded CLN2 Disease Clinical Rating Scale-Motor (CRS-MX) is a performance measure designed to assess the full range of a participant's ability to ambulate. The original CLN2 CRS Motor was expanded to have increased granularity and improved ability to capture a wider range of ambulatory functional levels. The new 7-point rating scale has a range from 0 to 6, with 0 indicating no independent locomotion and 6 indicating normal gait in the home and community environment without any ataxia or pathologic falls.

The Expanded CLN2 Disease Clinical Rating Scale-Language (CRS-LX) is a clinician-reported item designed to assess the full range of the participant's use of expressive language. The CLN2 CRS Language was expanded to have increased granularity and improved ability to capture a wider range of language levels. Caregiver report in an Advisory Panel supported that heterogeneity is present in peak level of expressive language and that adequate response options should reference expressive language expectations by age. In addition, although children with CLN2 experience a progressive decline in the number of words expressed they continue to communicate using vocalization and gestures. Therefore, the modified CLN2 CRS-LX also includes response options with use of vocalization/jargon and gestures.

In some embodiments, a method of treating CLN2 Disease described herein results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline (i.e., pre-treatment value) as measured by the combined Motor and Language domains of the CLN2 CRS. In some embodiments, a method of treating CLN2 Disease described herein results in a clinical improvement of 1, 2, 3, 4, 5, or 6 categories compared to baseline as measured by the combined Motor and Language domains of the CLN2 CRS. In some embodiments, a method of treating CLN2 Disease described herein results in a decline of less than 4, less than 3, or less than 2 categories from baseline on the combined Motor and Language domains of the CLN2 CRS at about 3 months, about 6 months, about 9 months, about 12 months, about 15 months, about 18 months, about 21 months, about 24 months, about 27 months, about 30 months, about 33 months, about 36 months, about 39 months, about 42 months, about 45 months, about 48 months, about 51 months, about 54 months, about 57 months, about 60 months, about 63 months, about 66 months, about 69 months, or at about 72 months after treating.

In some embodiments, a method of treating CLN2 Disease described herein results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the Language domain of the CLN2 CRS. In some embodiments, a method of treating CLN2 Disease described herein results in a clinical improvement of 1, 2, or 3 categories compared to baseline as measured by the Language domain of the CLN2 CRS. In some embodiments, a method of treating CLN2 Disease described herein results in a decline of less than 2 categories from baseline on the Language domain of the CLN2 CRS at about 3 months, about 6 months, about 9 months, about 12 months, about 15 months, about 18 months, about 21 months, about 24 months, about 27 months, about 30 months, about 33 months, about 36 months, about 39 months, about 42 months, about 45 months, about 48 months, about 51 months, about 54 months, about 57 months, about 60 months, about 63 months, about 66 months, about 69 months, or at about 72 months after treating.

In some embodiments, a method of treating CLN2 Disease described herein results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the Motor domain of the CLN2 CRS. In some embodiments, a method of treating CLN2 Disease described herein results in a clinical improvement of 1, 2, or 3 categories compared to baseline as measured by the Motor domain of the CLN2 CRS. In some embodiments, a method of treating CLN2 Disease described herein results in a decline of less than 2 categories from baseline on the Motor domain of the CLN2 CRS at about 3 months, about 6 months, about 9 months, about 12 months, about 15 months, about 18 months, about 21 months, about 24 months, about 27 months, about 30 months, about 33 months, about 36 months, about 39 months, about 42 months, about 45 months, about 48 months, about 51 months, about 54 months, about 57 months, about 60 months, about 63 months, about 66 months, about 69 months, or at about 72 months after treating.

In some embodiments, the efficacy of a method of treating CLN2 Disease provided herein may be assessed by measuring the frequency, duration and/or type of seizures in a subject. Seizure data may be collected by a caregiver in an electronic diary (eDiary), e.g., using The Caregiver Seizure Diary App. For each observed seizure, the seizure type and the length of time the seizure lasted may be recorded.

In some embodiments, a method of treating CLN2 Disease described herein results in a reduction in the frequency of seizures of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as recorded in the Caregiver Seizure Diary.

In some embodiments, a method of treating CLN2 Disease described herein results in a reduction in the duration of seizures of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as recorded in the Caregiver Seizure Diary.

In some embodiments, a method of treating CLN2 Disease described herein result in a decrease in the use of antiepileptic treatments of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline.

In some embodiments, the Pediatric Quality of Life Inventory (PedsQL) Generic Core Scale may be used to assess the efficacy of a method of treatment described herein. The PedsQL is a QOL assessment scale for children (Varni J W. Scaling and Scoring of the Pediatric Quality of Life Inventory™ PedsQL™. Lyon, France: Mapi Research Trust; 2017). The Parent Proxy version of the Generic Core Scales includes physical, social, emotional and school functioning. The PedsQL Generic Core Scale is a 23-item questionnaire, rating performance on a 5-point scale of raw scores (0=“never a problem” to 4=“almost always a problem”) that are reverse scored and mapped to a scale of 0 to of 100, where higher scores indicate better health-related QOL. Derived scores for the Generic Core Scales will include the Total Scale Score, the Physical Health Summary Score and the Psychosocial Health Summary Score

The Family Impact Module (FIM) may be used to measure the impact of pediatric chronic health conditions on parents and the family, as scored on dimensions of physical, emotional, social, and cognitive functioning as well as communication, worry, daily activities, and family relationships. Caregivers rate each category from “never a problem” (0) to “always a problem” (4). Score calculation steps will change these scores to a scale of 0 to 100, where 0 is the worst score and 100 is the best. Derived scores for the FIM include the Total Score, the Parent Health Related Quality of Life (HRQL) Summary Score and the Family Functioning Summary Score.

In some embodiments, a method of treating CLN2 Disease described herein results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the Pediatric Quality of Life Inventory Generic Core Scale. In some embodiments, a method of treating CLN2 Disease described herein results in a clinical improvement of 1, 2, 3, 4, or 5 categories compared to baseline as measured by the Pediatric Quality of Life Inventory Generic Core Scale.

In some embodiments, a method of treating CLN2 Disease described herein results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the PedsQL Family Impact Module. In some embodiments, a method of treating CLN2 Disease described herein results in a clinical improvement of 1, 2, 3, or 4 categories compared to baseline as measured by the PedsQL Family Impact Module.

In some embodiments, the efficacy of efficacy of a method of treatment described herein may be assessed by measuring neurodevelopmental parameters of adaptive, cognitive, motor, language and behavioral function over time, using, e.g., the Vineland Adaptive Behavior Scales, 3rd Edition, Expanded Interview Form (VABS-III) and/or the Mullen Scales of Early Learning (MSEL). The VABS-III (Sparrow et al., Vineland Adaptive Behavior Scales. 3rd ed. Bloomington, MN: Pearson; 2016) assesses adaptive behavior in individuals from infancy to age 90 years. It is conducted by a trained clinician with a caregiver or person who is familiar with the participant. The scale may include including 4 domains of Communication, Daily Living Skills, Socialization, and Motor Skills will be assessed, as these are appropriate for children aged <7 years. Items in each of the domains are scored from 2 to 0, based on the frequency that the individual demonstrates each adaptive skill/behavior, with 2 corresponding to almost always and 0 to never. The individual items are tallied and then calculated as a composite score to be compared against a standard, age-matched bell curve with a mean of 100 and a standard deviation of 15. Each subdomain score also yields an adaptive behavior age equivalence score (ABAE). The mean ABAE score can be calculated by averaging all subdomain age equivalence scores except the motor subdomains.

The MSEL (available from: <https://www.pearsonclinical.com/childhood/products/100000306/mullen-scales-of-early-learning.html>). is a standardized clinical psychology assessment that is commonly used as a measure of cognitive development in young children. The MSEL is organized into 5 subscales: (a) gross motor, (b) fine motor, (c) visual reception (or non-verbal problem solving), (d) receptive language, and (e) expressive language. Each subscale is standardized to calculate a standard score, percentile and age-equivalent score.

In some embodiments, a method of treating CLN2 Disease described herein results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by the Vineland Adaptive Behavior Scale III.

In some embodiments, a method of treating CLN2 Disease described herein results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by the Mullen Scale of Early Learning.

In some embodiments, the DEM-CHILD: CLN2 Movement Disorder Inventory may be used to assess the efficacy of a method of treating CLN2 Disease provided herein. The DEM-CHILD questionnaires may include CLN2 Movement Disorder Inventory and/or the CLN2 Disease-based QOL Assessment. The CLN2 Movement Disorder Inventory includes 7 questions about the frequency and severity of movement disorder events a participant experiences, broken down by type (myoclonus, dystonia, dysmetria, chorea, and tics/stereotypy). Each question is rated from 0 to 3, with 0 being marked severity/common frequency and 3 being none for severity/absent frequency. The CLN2 Disease-based Quality of Life (QOL) Assessment includes 28 questions rated on a 5-category scale of “never” (positive outcome) to “almost always” (negative outcome). Questions are broken into groups of seizures, feeding, sleep, behavior, and daily activities.

In some embodiments, a method of treating CLN2 Disease described herein results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined using the DEM-CHILD: CLN2 Movement Disorder Inventory.

In some embodiments, the efficacy of a method of treatment described herein may be assessed by measuring CNS structural abnormalities using MRI of the brain and/or measuring changes in retinal anatomy by SD-OCT over time. An MRI of the brain may be used to assess whole brain volume, gray matter volume, white matter volume, CSF volume, diffusion tensor for visualization of the optic tracts, and whole-brain apparent diffusion coefficient. MRI will be performed with gadolinium. MRI of the lumbar and lumbosacral spinal cord may be used to assess dorsal column lesions. SD-OCT using the Heidelberg Spectralis OCT instrument with Flex Module may be used to assess retinal anatomy.

In some embodiments, a method of treating CLN2 Disease described herein results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by assessing retinal anatomy using Spectral Domain Optical Coherence Tomography (SD-OCT).

In some embodiments, the efficacy of a method of treatment described herein may be assessed using Clinician Global Impression of Change (Cl-GIC) and/or Clinician Global Impression of Severity (Cl-GIS). The Cl-GIS is an 8-question instrument performed by a clinician to track changes in the severity of participants' CLN2 disease over time for the parameters of seizure, cognitive function, motor, speech, involuntary/disordered movement, vision, ability to swallow/eat, as well as overall disease. The Cl-GIS has a 5-item scale that ranges from “none or no impairment” (score of 1) to “severe or severe impairment” (score of 5). The final question of the Cl-GIS is the Cl-GIC. The Cl-GIC assesses the overall change the clinician has observed in the participant between assessments. The 5-item scale ranges from “much better” (score of 1) to “much worse” (score of 5).

In some embodiments, a method of treating CLN2 Disease described herein results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by CI-GIS. In some embodiments, a method of treating CLN2 Disease described herein results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by CI-GIC. In some embodiments, a method of treating CLN2 Disease described herein results in a clinical improvement of 1, 2, 3, 4, or 5 categories compared to baseline as measured by CI-GIC.

In another aspect, a method of treating CLN2 Disease described herein may be assessed by measuring gait abnormalities in a subject, e.g., by using GAITRite. The GAITRite System is an electronic walkway utilized to measure the temporal (timing) and spatial (two dimension geometric position) parameters of its pressure activated sensors. The GAITRite system can be used as a measuring device to assess biped ambulatory capacity of a subject. In some embodiments, a method of treating CLN2 Disease described herein results in an improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% in gait parameters compared to baseline as determined by GAITRite.

In certain embodiments, the methods of treating CLN2 Disease provided herein may result in an increased TPP1 activity in the spinal cord of said subject. In certain embodiments, the methods of treating CLN2 Disease provided herein comprise may result in a TPP1 activity in the spinal cord of said subject that is at least 2%, 3%, 5%, 6%, 7%, 8%, 9%,10%, 12%, 15%, 17%, 20%, 25%, 30%, 35%, 40%, 45%, or 50% higher than a reference TPP1 activity in the spinal cord of a second subject, and wherein the reference TPP1 activity in the spinal cord is measured when said second subject does not receive the treatment using said method, and wherein said second subject is the same or different from said subject. In certain embodiments, the methods of treating CLN2 Disease provided herein comprise may result in a TPP1 activity in the spinal cord of said subject that is 2%, 3%, 5%, 6%, 7%, 8%, 9%,10%, 12%, 15%, 17%, 20%, 25%, 30%, 35%, 40%, 45%, or 50% higher than a reference TPP1 activity in the spinal cord of a second subject, and wherein the reference TPP1 activity in the spinal cord is measured when said second subject does not receive the treatment using said method, and wherein said second subject is the same or different from said subject.

In certain embodiments, the methods of treating CLN2 Disease provided herein may result in an increased hepatic TPP1 activity of said subject. In certain embodiments, the methods of treating CLN2 Disease provided herein comprise may result in a hepatic TPP1 activity of said subject that is at least 2%, 3%, 5%, 6%, 7%, 8%, 9%, 10%, 12%, 15%, 17%, 20%, 25%, 30%, 35%, 40%, 45%, or 50% higher than a reference hepatic TPP1 activity in a second subject, and wherein the reference hepatic TPP1 activity is measured when said second subject does not receive the treatment using said method, and wherein said second subject is the same or different from said subject. In certain embodiments, the methods of treating CLN2 Disease provided herein comprise may result in a hepatic TPP1 activity of said subject that is 2%, 3%, 5%, 6%, 7%, 8%, 9%10%, 12%, 15%, 17%, 20%, 25%, 30%, 35%, 40%, 45%, or 50% higher than a reference hepatic TPP1 activity in a second subject, and wherein the reference hepatic TPP1 activity is measured when said second subject does not receive the treatment using said method, and wherein said second subject is the same or different from said subject.

In certain embodiments, the methods of treating CLN2 Disease provided herein may result in an increased serum TPP1 activity of said subject. In certain embodiments, the methods of treating CLN2 Disease provided herein comprise may result in a serum TPP1 activity of said subject that is at least 2%, 3%, 5%, 6%, 7%, 8%, 9%, 10%, 12%, 15%, 17%, 20%, 25%, 30%, 35%, 40%, 45%, or 50% higher than a reference serum TPP1 activity in a second subject, and wherein the reference serum TPP1 activity is measured when said second subject does not receive the treatment using said method, and wherein said second subject is the same or different from said subject. In certain embodiments, the methods of treating CLN2 Disease provided herein comprise may result in a serum TPP1 activity of said subject that is 2%, 3%, 5%, 6%, 7%, 8%, 9%, 10%, 12%, 15%, 17%, 20%, 25%, 30%, 35%, 40%, 45%, or 50% higher than a reference serum TPP1 activity in a second subject, and wherein the reference serum TPP1 activity is measured when said second subject does not receive the treatment using said method, and wherein said second subject is the same or different from said subject.

In certain embodiments, the methods of treating CLN2 Disease provided herein may result in a reduced microglial activity in the cortex of said subject. In certain embodiments, the methods of treating CLN2 Disease provided herein comprise may result in a microglial activity in the cortex of said subject that is at least 2%, 3%, 5%, 6%, 7%, 8%, 9%,10%, 12%, 15%, 17%, 20%, 25%, 30%, 35%, 40%, 45%, or 50% lower than a reference microglial activity in the cortex in a second subject, and wherein the reference microglial activity in the cortex is measured when said second subject does not receive the treatment using said method, and wherein said second subject is the same or different from said subject. In certain embodiments, the methods of treating CLN2 Disease provided herein comprise may result in a microglial activity in the cortex of said subject that is 2%, 3%, 5%, 6%, 7%, 8%, 9%, 10%, 12%, 15%, 17%, 20%, 25%, 30%, 35%, 40%, 45%, or 50% lower than a reference microglial activity in the cortex in a second subject, and wherein the reference microglial activity in the cortex is measured when said second subject does not receive the treatment using said method, and wherein said second subject is the same or different from said subject.

In certain embodiments, the methods of treating CLN2 Disease provided herein may result in an increase TPP1 activity in the brain of said subject. In certain embodiments, the methods of treating CLN2 Disease provided herein may result in a TPP1 activity in the brain of said subject that is at least 2%, 3%, 5%, 6%, 7%, 8%, 9%, 10%, 12%, 15%, 17%, 20%, 25%, 30%, 35%, 40%, 45%, or 50% higher than a reference TPP1 activity in the brain of a second subject, wherein the reference TPP1 activity in the brain is measured when said second subject does not receive the treatment using said method, and wherein said second subject is the same or different from said subject. In certain embodiments, the methods of treating CLN2 Disease provided herein may result in a TPP1 activity in the brain of said subject that is 2%, 3%, 5%, 6%, 7%, 8%, 9%, 10%, 12%, 15%, 17%, 20%, 25%, 30%, 35%, 40%, 45%, or 50% higher than a reference TPP1 activity in the brain of a second subject, wherein the reference TPP1 activity in the brain is measured when said second subject does not receive the treatment using said method, and wherein said second subject is the same or different from said subject.

In certain embodiments, the method results in a TPP1 activity in the cerebral spinal fluid of the subject that is at least about 50%, at least about 75%, at least about 80%, at least about 90%, or about the same, or greater than 100% of the biological activity level of the native TPP1 protein, or a natural variant or polymorph thereof which is not associated with disease. In certain embodiments, the method results in a serum TPP1 activity of the subject that is at least about 50%, at least about 75%, at least about 80%, at least about 90%, or about the same, or greater than 100% of the biological activity level of the native TPP1 protein, or a natural variant or polymorph thereof which is not associated with disease.

The skilled artisan may use the assays as described herein and/or techniques known in the art (for example, assays described in WO2018209205A1) to study the composition and methods described herein, for example to study the rAAV provided herein in method of treating CLN2 Disease.

Related assays may include but are not limited to the following: in vivo study in TPP1^{m1j} mice model for CLN2 Disease, natural history study of TPP1^{m1j} knock out mice, pharmacology study in TPP1^{m1J} KO Mice, assays measuring TPP1 enzyme activity, assessment of intracerebroventricular efficacy in mice using non-invasive full time monitoring in a digital vivarium, measuring effects of TPP1 replacement using AAV9 Delivery (ICV) in C57BL/6 TPP1m1J KO mice, safety pharmaceutical assays, toxicity study in mice, and pharmacodynamic studies in cynomolgus monkeys, assays for vector biodistribution, assays for vector shedding, repeat dose studies, carcinogenicity studies, and other toxicity studies.

In some embodiments, disease progression may be assessed by administration of CLN2 CRS-MX to pediatric patients. In some embodiments, disease progression may be assessed by administration of CLN2 CRS-MX to adult patients. In some embodiments, provided herein is a method of treating CLN2 disease due to TPP1 deficiency in a subject comprising administering to the central nervous system of the subject in need thereof 1.25×10¹¹ or 4.5×10¹¹ genome copies per gram brain mass of a recombinant adeno-associated virus (rAAV) into the central nervous system (CNS), wherein said recombinant adeno-associated virus (rAAV) comprises an AAV capsid and a vector genome packaged therein, and wherein said vector genome comprises
an AAV 5′ inverted terminal repeat (ITR) sequence;
a promoter;
a CLN2 coding sequence encoding a human TPP1; and
an AAV 3′ ITR;
   wherein the method further comprises monitoring changes, or lack thereof, in said patient's CLN2 CRS-MX rating during and/or following administration of the vector. In some embodiments, the subject has a change from baseline in their CLN2 CRS-MX rating of +1 point, +2 points, +3 points, +4 points, +5 points, or +6 points. In some embodiments, the method slows or arrests progression of ocular manifestations associated with CLN2 Batten disease in a subject, determined by a slowed decrease in and/or maintenance of the subject's CLN2 CRS-MX rating over a period of 1 month or more, 2 months or more, 3 months or more, 6 months or more, 1 year or more, or 2 years or more.

In some embodiments, disease progression may be assessed by administration of CLN2 CRS-LX to pediatric patients. In some embodiments, provided herein is a method of treating In some embodiments, provided herein is a method of treating CLN2 disease due to TPP1 deficiency in a subject comprising administering to the central nervous system of the subject in need thereof 1.25×10¹¹ or 4.5×10¹¹ genome copies per gram brain mass of a recombinant adeno-associated virus (rAAV) into the central nervous system (CNS), wherein said recombinant adeno-associated virus (rAAV) comprises an AAV capsid and a vector genome packaged therein, and wherein said vector genome comprises
an AAV 5′ inverted terminal repeat (ITR) sequence;
a promoter;
a CLN2 coding sequence encoding a human TPP1; and
an AAV 3′ ITR;
   wherein the method further comprises monitoring changes, or lack thereof, in said patient's CLN2 CRS-LX rating during and/or following administration of the vector. In some embodiments, the subject has a change from baseline in their CLN2 CRS-LX rating of +1 point, +2 points, +3 points, +4 points, +5 points, or +6 points. In some embodiments, the method slows or arrests progression of ocular manifestations associated with CLN2 Batten disease in a subject, determined by a slowed decrease in and/or maintenance of the subject's CLN2 CRS-LX rating over a period of 1 month or more, 2 months or more, 3 months or more, 6 months or more, 1 year or more, or 2 years or more.

### 6.4 Combination Therapies

The methods described herein can also be combined with any other therapy for treatment of CLN2 Disease or the symptoms thereof. The management of CLN2 disease is complex. Patients require extensive multidisciplinary medical care due to the high symptom load and the rapid rate of functional decline, and families require extensive psychosocial support, yet no management guidelines currently exist for this condition. See, e.g., Williams et al, Management strategies for CLN2 disease, Pediatric Neurology 69 (2017) 102e112, which is incorporated herein by reference. However, in certain embodiments, the standard of care may include intracerebroventricular cerliponase alpha (BMN 190). See, Schulz et al, Intracerebroventricular cerliponase alfa (BMN 190) in children with CLN 2 disease: results from a phase 1/2 open label, dose-escalation study, J Inherit Metab Disease, 39:S51, which is incorporated herein by reference. The recommended dosage is 30-300 mg ICV infusion administered every other week.

In some embodiments, a method of treating CLN2 Disease provided herein further comprises administering immunotherapy to a subject. Examples of immunotherapies that may be administered include corticosteroids, tacrolimus and sirolimus.

### 7. EXAMPLES

The Example in this section (i.e., section 5, is provided for purposes of illustration and is not intended to limit the invention.

### 7.1 Example 1: Safety Pharmacology

### 7.1.1 Central Nervous System

### (a) Three-Month Toxicity Study in Mice

Functional Observational Battery (FOB) evaluations were conducted in Week 13 and included an evaluation of activity, posture, rearing, behavior, response to stimulus (approach, click, tail pinch, and touch), pupil response, grip response and pain perception (latency of response to a nociceptive [thermal]) stimulus). There were no effects on these parameters, with the only finding of note being a decrease in the numbers of rears within the open field in males at >2.0×10¹¹ GC/animal and females at 8.5×10¹¹ GC/animal. In the absence of other findings, this was not considered to be Construct III-related.

### (b) Four-Week Pharmacodynamic Study in Cynomolgus Monkeys

In the 4-week study in cynomolgus monkeys, in addition to clinical observations, a comprehensive neurological examination in Week 4 that included general sensory and motor function, cerebral reflexes (pupillary, orbicularis and corneal reflexes) and spinal reflexes (sensory, knee jerk, cutaneous, proprioceptive and tail reflexes) was conducted. There were no Construct III-related effects on these endpoints and no animal had behavioral abnormalities or clinical signs during the study.

There were no CNS-related effects observed in the 4-week investigative toxicity study in cynomolgus monkeys.

### 7.1.2 Respiratory System

There were no respiratory changes observed in NHP following administration of Construct III. There were no effects on the respiratory endpoints assessed in the FOB in the mouse toxicity study (Construct III.) or microscopic changes in the lungs after 4 or 13 weeks.

In the 4-week study in cynomolgus monkeys, there were no effects noted on respiration that was included as an endpoint in the neurological assessment conducted in Week 4.

### 7.1.3 Cardiovascular System

There were no microscopic changes in the mouse toxicity study after 4 or 13 weeks of treatment with Construct III.

In the 4-week study in cynomolgus monkeys, there were no effects noted on heart rate that was included as an endpoint in the neurological assessment conducted in Week 4.

### 7.2 Example 2: A Single Dose Pharmacodynamic Study Via Intrathecal Administration in Cynomolgus Monkeys

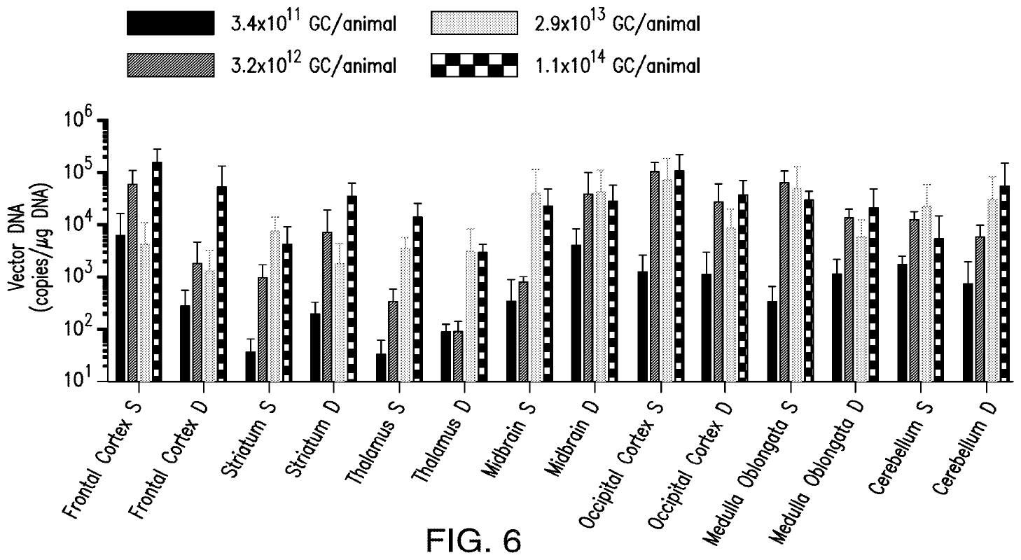

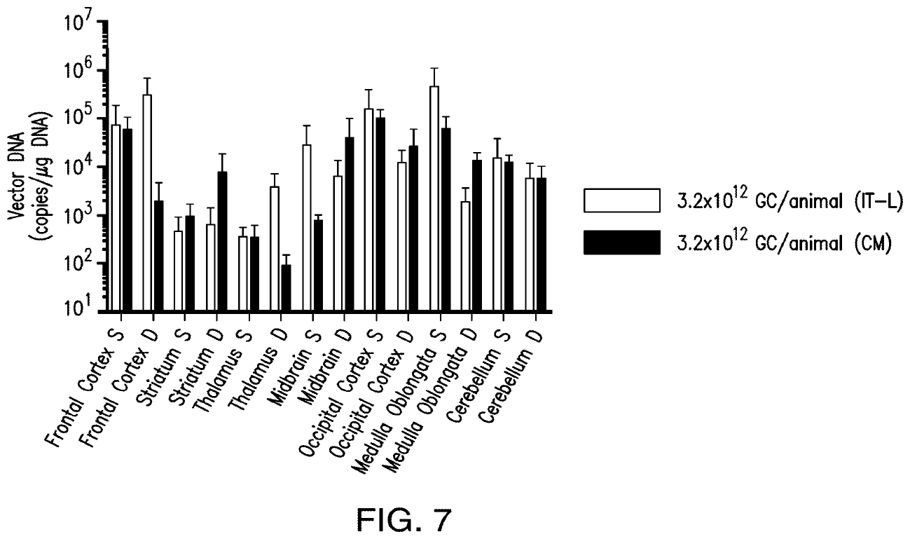

In the 4-week pharmacodynamic and toxicity study in cynomolgus monkeys with Construct III, samples were collected from the brain (two 4-mm round samples, one superficial and one deep, of frontal cortex, occipital cortex, cerebellum, striatum, medulla oblongata, midbrain and thalamus), spinal cord (1-cm segment of cervical, thoracic and lumbar sections) and liver. Groups of cynomolgus monkeys (1 male and 2 females/group) were administered Construct III via intrathecal injection via cisterna magna (CM) puncture at doses of 0, 3.4×10¹¹, 3.2×10¹² or 2.9×10¹³ genome copies (GC)/animal (1 mL/animal). An additional group of animals (1 male and 2 females) was administered 3.2×10¹² GC/animal via intrathecal-lumbar puncture (IT-L; 1 mL/animal). At the end of the study, animals were euthanized on Day 29. Overall, vector DNA levels were above the lower limit of quantification in almost all brain regions, trigeminal ganglion, liver, sciatic nerve and spinal cord tissues collected from animals administered Construct III via cisterna magna or IT-L administration. The biodistribution results are summarized in FIG. 6, FIG. 7 and Table 2.

### 7.3 Example 3: A Single Dose Intrathecal (IT) Pharmacokinetic/Pharmacodynamic Study in Cynomolgus Monkeys

In the 4-week pharmacodynamic and toxicity study in cynomolgus monkeys with Construct III, samples were collected from the brain (two 4-mm round samples, one superficial and one deep, of frontal cortex, occipital cortex, cerebellum, striatum, medulla oblongata, midbrain and thalamus), spinal cord (1-cm segment of cervical, thoracic and lumbar sections), and the associated nerve roots and ganglia (DRG), eye (left), heart (left ventricle), kidney (left), liver (left lateral lobe), lung (left caudal), proximal sciatic nerve, lymph nodes (inguinal, mandibular and mesenteric), ovary (left) or testis. The eye tissue was further dissected into retina/choroid and sclera. Groups of cynomolgus monkeys (2 males and 2 females/dose) were administered Construct III (AAV9.hCLN2) via a single cisterna magna puncture (CM) at doses of 0, 3.1×10¹³ or 1.1×10¹⁴ genome copies (GC)/animal. At 3.1×10¹³ GC/animals, Construct III was prepared using three different methods. An additional group of animals (n=2/sex/group) was administered AAV9 with a null vector at dose of 2.89×10¹³ GC/animal via a single CM administration. At the end of the study, animals were euthanized on Day 30. Overall, vector DNA levels were above the lower limit of quantification in almost all brain regions, spinal cord and DRG tissues, proximal sciatic nerve and other peripheral tissues collected from animals administered Construct III or null vector on Day 30. The biodistribution results can be found in FIG. 6, FIG. 7, and Table 2.

Groups of cynomolgus monkeys (n=3/group or n=4/group) were administered Construct III (AAV9.hCLN2) via injection into the cisterna magna (CM) at doses of 3.4×10¹¹, 3.2×10¹², 2.9×10¹³ GC/animal or 1.1×10¹⁴ GC/animal. At necropsy, two tissue punches were collected for analysis by qPCR from either the deep (>3 mm; D) or superficial (<3 mm deep; S) areas of frontal cortex, striatum, thalamus, midbrain, occipital cortex, medulla oblongata and cerebellum. Mean and standard deviations are shown. BLQ values were treated as 50.0 copies/μg DNA in the calculation of mean.

Groups of cynomolgus monkeys (n=3/dose) were administered Construct III (AAV9.hCLN2) via injection into the cisterna magna (CM) or via IT-lumbar (IT-L) at a dose of 3.2×10¹² GC/animal. At necropsy, two tissue punches were collected for analysis by qPCR from either the deep (>3 mm; D) or superficial (<3 mm deep; S) areas of frontal cortex, striatum, thalamus, midbrain, occipital cortex, medulla oblongata and cerebellum. Mean and standard deviations are shown. BLQ values were treated as 50.0 copies/g DNA in the calculation of mean.

TABLE 2
Summary of Vector DNA Biodistribution in cynomolgus monkeys
administered with Construct III (AAV9.hCLN2) via injection
into the cisterna magna (CM) or via IT-lumbar (IT-L)

| | | Dose (GC/animal) | | | | |
|---|---|---|---|---|---|---|
| | | 3.2 × 10¹² | 3.4 × 10¹¹ | 3.2 × 10¹² | 2.9 × 10¹³ | 1.1 × 10¹⁴ |
| | | (IT-L) | (CM) | (CM) | (CM) | (CM) |
| | | Vector DNA copies/μg DNA | | | | |
| Frontal cortex | Superficial | 7.57E+04 | 6.44E+03 | 6.01E+04 | 5.27E+04 | 1.55E+05 |
| | Deep | 3.09E+05 | 2.79E+02 | 1.95E+03 | 4.22E+03 | 5.50E+04 |
| Striatum | Superficial | 4.86E+02 | 3.61E+01 | 1.01E+03 | 1.30E+03 | 4.34E+03 |
| | Deep | 6.59E+02 | 1.95E+02 | 7.88E+03 | 7.65E+03 | 3.57E+04 |
| Thalamus | Superficial | 3.77E+02 | 3.33E+01 | 3.55E+02 | 1.83E+03 | 1.42E+04 |
| | Deep | 4.05E+03 | 9.08E+01 | 9.24E+01 | 3.50E+03 | 2.91E+03 |
| Midbrain | Superficial | 2.82E+04 | 3.60E+02 | 7.97E+02 | 3.31E+03 | 3.18E+04 |
| | Deep | 7.12E+03 | 4.18E+03 | 4.01E+04 | 4.29E+04 | 2.92E+04 |
| Occipital | Superficial | 1.62E+05 | 1.32E+03 | 1.06E+05 | 7.41E+04 | 1.14E+05 |
| Cortex | Deep | 1.24E+04 | 1.16E+03 | 2.79E+04 | 8.75E+03 | 3.95E+04 |
| Medulla | Superficial | 4.90E+05 | 3.56E+02 | 6.27E+04 | 5.08E+04 | 3.18E+04 |
| Oblongata | Deep | 2.00E+03 | 1.18E+03 | 1.39E+04 | 6.27E+03 | 2.23E+04 |
| Cerebellum | Superficial | 1.58E+04 | 1.78E+03 | 1.24E+04 | 2.40E+04 | 5.22E+03 |
| | Deep | 6.09E+03 | 7.43E+02 | 6.09E+03 | 3.14E+04 | 5.52E+04 |
| Spinal Cord | Cervical | 2.81E+05 | 1.36E+04 | 4.93E+02 | 1.61E+05 | 4.64E+04 |
| | Thoracic | 1.26E+05 | 1.18E+04 | 3.65E+02 | 1.91E+05 | 1.51E+05 |
| | Lumbar | 9.68E+03 | 1.36E+05 | 3.71E+05 | 1.55E+05 | 2.12E+05 |

| | | | | | |
|---|---|---|---|---|---|
| Liver | 7.20E+06 | 7.20E+06 | 2.02E+06 | 6.30E+06 | 5.28E+07 |
| Trigeminal ganglion | 5.79E+04 | 5.79E+04 | 5.84E+04 | 5.22E+05 | NA |
| Sciatic nerve | 2.32E+04 | 2.32E+04 | 7.87E+03 | 2.72E+04 | 2.30E+03 |

BLQ values were treated as 50.0 copies/μg DNA in the calculation of mean.
CM = cisterna magna; IT-L = intrathecal-lumbar; NA = Not collected.

### 7.4 Example 4: A Single Dose Pharmacology Study in TPP1m1J KO Mice

The objective of this study was to evaluate the pharmacology (clinical signs, neuropathology and survival) of Construct III in TPP1^{m1J} KG mice following a single ICV dose. At the end of the study, additional anatomic pathology evaluation of the spinal cord was conducted in surviving animals. Groups of TPP1^{m1J} KG mice (9-10/sex/group; 4-5 weeks old) were administered a single ICV injection (5 μL) of Construct III at doses of 0 (vehicle), 1.25×10¹⁰, 5.0×10¹⁰, 2.0×10¹¹, and 8.5×10¹¹ GC/animal. Animals were genotyped prior to allocation and at the end of the study.

Endpoints evaluated in this study included: mortality, clinical observations, body weight, neurobehavioral observations (predose, Week 8, and Week 16), TPP1 activity, anti-TPP1 antibody analysis, gross necropsy findings, organ weights, and neuropathology.

Essentially, a clear effect of Construct III on neurobehavior was not observed because a dose response was not observed or control knock-out animals did not survive to be assessed at later time points. In this study, there were no Construct III-related adverse findings in surviving mice up until 52 weeks at the minimum effective dose for survival (8.5×10¹¹ GC/animal). Doses of 1.25×10¹⁰ and 5.0×10¹⁰ GC/animal did not appear to increase the survival of TPP1^{m1J} KO mice when compared to the untreated TPP1^{m1J} KO mice. At 2.0×10¹¹ GC/animal, it was not possible to determine whether or not Construct III prolonged survival as 4/5 males and 2/5 females were heterozygous for the TPP1 gene when genotyped at the end of the study due to supplier error. At this dose, the one confirmed male TPP1^{m1J} KO mouse survived until the end of the study. In all animals receiving Construct III at 8.5×10¹¹ GC/animal, there was 100% survival in both males and females to the scheduled necropsy of 52 weeks.

There were no macroscopic findings at the Week 9 interval in any animals. At 52 Weeks, a mass on the liver was observed at 2×10¹¹ GC/animal (1/5 males; heterozygous) and 8.5×10¹¹ GC/animal (3/5 males and 1/5 females TPP1^{m1J} KO mice). In the liver, hepatocellular adenoma (1/5 males and 1/5 females at 8.5×10¹¹ GC/animal and 1/5 males at 2×10¹¹ GC/animal), hepatocyte necrosis (3/5 males at 8.5×10¹¹ GC/animal and 1/5 males at 2×10¹¹ GC/animal), hepatocyte hyperplasia (4/5 males at 8.5×10¹¹ GC/animal and 1/2 females at 2×10¹¹ GC/animal) and an increased severity of hepatocyte vacuolation were observed. In the 52-Week cohort, microscopic changes were observed in dorsal root ganglia and spinal nerve roots following administration of Construct III. In the dorsal root ganglia, neuronal vacuolation (minimal to marked) was noted in mice at ≥5.0×10¹⁰ GC/animal and increased cellularity (likely of glial cells, minimal to moderate) and axonal dystrophy/swelling (minimal to mild) were seen in mice at ≥2.0×10¹¹ GC/animal. Degeneration (minimal to moderate) and axonal dystrophy/swelling (minimal to mild) in the spinal roots were noted in mice at ≥5.0×10¹⁰ GC/animal. Due to a lack of vehicle control TPP1^{m1J} KO mice surviving to the 52-week necropsy, it was not possible to evaluate livers of TPP1 m^{m1J} KO mice not administered Construct III; thus, differentiation between CONSTRUCT III-related and phenotype-related lesions was not possible.

### 7.5 Example 5: Three-Month Toxicity Study in C57B1/6 Mice

The objective of this study was to evaluate the pharmacodynamics and immunogenicity of Construct III in C57B1/6 mice following a single ICV dose. Groups of mice (n=30/sex/group) were administered a single ICV injection (5 μL) of Construct III at doses of 0 (vehicle), 1.25×10¹⁰, 5.0×10¹⁰, 2.0×10¹¹, and 8.5×10¹¹ GC/animal. Animals were euthanized after either 4 (10/sex/group) or 13 (10/sex/group) weeks after dosing. An additional group of satellite animals (n=5/sex/group) was euthanized at each time-point to evaluate transgene product (TPP1 activity) in the brain and liver. Compatibility testing using the exact dosing apparatus showed some vector loss at the lower doses, therefore doses administered were 0.9×10⁹ (70% recovery), 3.9×10¹⁰ (77% recovery), 1.8×10¹¹ GC/animal (90% recovery) and 8.5×10¹¹ GC/animal (100% recovery) for 1.25×10¹⁰, 5.0×10¹⁰, 2.0×10¹¹, and 8.5×10¹¹ GC/animal, respectively.

Endpoints included: TPP1 enzyme activity (serum, brain and liver) and serum anti-TPP1 antibodies (ATPA).

Administration of Construct III to mice led to dose dependent increases in brain TPP1 activity in males and females with no sex-related differences and no differences between Weeks 4 and 13 in both sexes. Liver TPP1 activity was increased in males and females in both Week 4 and 13. In males there were no differences between Week 4 and 13; however, in females, liver TPP1 activity was consistently lower in Week 13 than Week 4. At the highest dose, 8.5×10¹¹ GC/animal, liver TPP1 activity was 18-(males) and 4 (females)—fold higher than brain TPP1 activity in Week 13. A dose dependent increase in serum TPP1 activity was observed in males and females; however, the values were highly variable. In Week 13, serum TPP1 activity was generally higher in males than females across all dose groups with an approximately 9.5-fold increase between males and females at 8.5×10¹¹ GC/animal. Serum TPP1 activity in Week 13 was decreased in males and increased in females when compared to Week 4. The majority of Construct III-treated animals were positive for ATPA in Week 4 and 13. The highest ATPA response was observed at the low dose (1.25×10¹⁰ GC/animal) when compared to the high dose (8.5×10¹¹ GC/animal). The difference between the low and high dose is of unknown significance as it may be attributable to interference in the assay by high transgene product concentrations or reflect an induction of immune tolerance.

### 7.6 Example 6: A Single Dose Pharmacodynamic Study Via Intrathecal Administration in Cynomolgus Monkeys

The objective of this study was to evaluate the pharmacodynamics of Construct III after intrathecal injection via cisterna magna puncture or intrathecal lumbar puncture in cynomolgus monkeys after 4 weeks.

Groups of cynomolgus monkeys (1 male and 2 females/group) were administered Construct III via intrathecal injection via cisterna magna puncture (CM) at doses of 0, 3.4×10¹¹, 3.2×10¹² or 2.9×10¹³ genome copies (GC)/animal (1 mL/animal). An additional group of animals (1 male and 2 females) were administered 3.2×10¹² GC/animal via intrathecal-lumbar puncture (IT-L; 1 mL/animal). At the end of the study, animals were euthanized on Day 29. Prior to dosing, all animals were negative for the presence of anti-AAV9 neutralizing antibodies (NAbs).

Serum and CSF samples collected from animals administered vehicle control article, 2.9×10¹³ GC/animal, or 3.2×10¹² GC/animal on Days 4, 15, and 29 were analyzed for inflammatory and neurodegeneration markers using Luminex technology and Simoa technology at Quanterix. The biomarkers included: amyloid beta isoform 40 (Aβ40), Aβ42, protein deglycase DJ-1, fibroblast growth factor 2, glial cell-derived neurotrophic factor, glial fibrillary acidic protein, granulocyte colony stimulating factor, granulocyte macrophage colony stimulating factor, interferon gamma, interleukin (IL) 1 alpha, IL-1β, IL-1 receptor agonist, IL-2, IL-4, IL-5, IL-6, IL-8, IL-10, IL-12, IL-13, IL-15, IL-16, IL-17A, IL-25, IL-18, IL, 21-IL-22, IL-23, IL 28A, IL-31, IL-33, interferon gamma induced protein 10, monocyte chemoattractant protein 1, macrophage inflammatory protein (MIP) 1 alpha, MIP-10, MIP-3α, neurofilament light, neuron specific enolase, chemokine ligand 5 (regulated on activation normal T cell expressed and secreted), S100 calcium binding protein B, soluble CD40 (cluster of differentiation 40) ligand, soluble Fas ligand, soluble receptor for advanced glycation end products, transforming growth factor alpha, tumor necrosis factor (TNF) alpha, TNFβ, ubiquitin carboxy-terminal hydrolase L1, and vascular endothelial growth factor.

There were no treatment-related clinical signs, effects on body weight, or physical or neurological examinations observed during the study.

Prior to dosing, all animals were negative for the presence of anti-AAV9 neutralizing antibodies. On Day 29, 12 samples were positive for the presence of anti-AAV9 neutralizing antibodies (Nab). The incidence of positive animals on Day 29 was 0/3 animals at 0 GC/animal, 3/3 animals at 3.4×10¹¹ GC/animal (CM), 3/3 animals at 3.2×10¹² GC/animal (CM), 3/3 animals at 2.9×10¹³ GC/animal (CM) and 3/3 animals at 3.2×10¹² GC/animal (IT-L).

Six animals out of 15 (40%) were deemed positive for anti-transgene product (TPP1) antibody (ATPA) responses at the pre-dose serum sample time-point. On Day 29, the incidence of ATPA positive animals was 1/3 animals at 0 GC/animal, 2/3 animals at 3.4×10¹¹ GC/animal (CM), 3/3 animals at 3.2×10¹² GC/animal (CM), 3/3 animals at 2.9×10¹³ GC/animal (CM) and 2/3 animals at 3.2×10¹² GC/animal (IT-L).

When compared to control animals, dose-related increases in TPP1 activity and concentration were observed in the CSF and serum at 2.9×10¹³ GC/animal (CM) with peak levels seen on Day 15. Thereafter, there was a trend for a decline in TPP1 towards the end of the study. Minimal increases in TPP1 activity and concentration were seen in the serum at 3.2×10¹² GC/animal (CM). At doses of ≥3.2×10¹² GC/animal (CM), increases in TPP1 activity and concentration were seen in the spinal cord (cervical, thoracic and lumbar) and liver when compared to the control group. While TPP1 activity did not show differences between different regions of the spinal cord, TPP1 concentrations were greater in the lumbar region of the spinal cord, compared to cervical or thoracic regions. At 2.9×10¹³ GC/animal (CM), TPP1 activity and concentration were increased in both the deep and superficial brain regions. At 3.2×10¹² GC/animal (CM), trends for small elevations in most of the deep brain regions when compared to the control mean value were observed for one animal only. At 3.4×10¹¹ GC/animal (CM), TPP1 activity and concentration were increased in the spinal cord (cervical, thoracic and lumbar regions) and liver only.

At 3.2×10¹² GC/animal (IT-L), whilst there were no clear differences in serum or CSF TPP1 activity, occasional increases in TPP1 concentration were observed when compared to the control group over the course of the study. A trend was observed for minimal increases in TPP1 activity and concentration in some brain regions, but this was minimal and due to small increases in one or two animals. The relationship of these changes to treatment is unclear as the increases were minimal and not observed in all animals. TPP1 activity and concentrations were increased in the spinal cord (cervical, thoracic and lumbar) and liver. In general, both TPP1 activity and concentration were greater in the spinal cord of IT-L treated animals, in particular the cervical and lumbar regions when compared to the IT-CM group at the same dose. The increase in the cervical region of spinal cord may be associated to animals in this group being placed in the Trendelenburg position immediately after dosing.

Six animals out of 15 were deemed positive for ATPA responses at the pre-dose serum sample time-point. On Day 29, the incidence of ATPA positive animals was 1/3 animals at 0 GC/animal, 2/3 animals at 3.4×10¹¹ GC/animal (CM), 3/3 animals at 3.2×10¹² GC/animal (CM), 3/3 animals at 2.9×10¹³ GC/animal (CM) and 2/3 animals at 3.2×10¹² GC/animal (IT-L).

On Day 29, all Construct III-treated animals were positive for the presence of serum anti-AAV9 NAbs antibodies. Vector DNA levels were above the lower limit of quantification in most brain regions, trigeminal ganglion, liver, sciatic nerve and spinal cord tissues collected from animals administered Construct III via cisterna magna or IT-L administration.

### 7.7 Example 7: Protocol for Treating Human Subjects and Assessing the Efficacy of Treatment with Construct III

This Example provides an exemplary protocol for the treatment of human subjects to allow an assessment of the efficacy of Construct III treatment. Construct III may be administered at a dose of about or at least about 1.25×10¹¹ and 4.5×10¹¹ gnome copies (GC)/g brain mass. Total dose administered (total GC) will be adjusted to account for differences in brain size. The total volume of product administered will not exceed 10% of the total CSF volume (estimated to be approximately 50 mL in infant brain and approximately 150 mL in an adult brain of approximately 1300 g).

Because of the relatively rapid brain growth that occurs early in a developing child, the total dose of Construct III administered IC or ICV depends on the estimated brain mass derived from the study participant's screening brain MRI. The study participant's estimated brain volume from his/her MRI will be converted to a brain mass and used to calculate the optimal dose to be administered.

### 7.7.1 Patient Population

Patients treated in accordance with the methods described in this example have a documented diagnosis of CLN2 disease due to TPP1 deficiency, confirmed by biochemical, molecular, or genetic methods. Patients may be male or female ≥4 months to ≤6 years of age on Day 1. If the patient is over 18 months of age, the patient has a screening CLN2 CRS score of at least 3 (using the 6-point combined Language and Motor domains).

If the patient is receiving enzyme replacement therapy (ERT; cerliponase alpha, BRINEURA), the caregiver or legal guardian is willing to suspend ERT at least 7 days prior to Construct III administration.

Patients who have had a heamatopietic stem cell transplant (HSCT) may be treated if this is deemed safe.

Patients should not be treated in accordance with the method described in this example if they have a contraindication for an IC or ICV injection, (e.g., a contraindication for an IC or ICV injection based on an MRI, a contraindication to general anesthesia, or a contraindication to MRI (or gadolinium)). Patients who have an estimated glomerular filtration rate (eGFR) of less than 30 mL/min/1.73 m² using measured creatinine should not be treated in accordance with the methods described herein.

Patients who have a history of a hypersensitivity reaction to tacrolimus, sirolimus, or prednisolone, or a history of a primary immunodeficiency (e.g., common variable immunodeficiency syndrome), splenectomy, or any underlying condition that predisposes the participant to infections should not be treated in accordance with the methods described herein. Patients should have absolute neutrophil counts of above 1×10³/L.

### 7.7.2 Objectives and Endpoints

Primary endpoints for efficacy is the proportion of participants without an unreversed (sustained) 2-category decline in the 6-point combined Motor and Language domains of the CLN2 Clinical Rating Scale (CRS) at 12, 18 and 24 Months. Other efficacy endpoints include:
Change from baseline in the combined Motor and Language domains of the CLN2
CRS at 12, 18 and 24 Months
Change from baseline in Motor domain of the CLN2 CRS at 12, 18 and 24 Months
Change from baseline in Language domain of the CLN2 CRS at 12, 18 and 24 Months
Change from baseline in the CLN2 CRS MX at 12, 18 and 24 Months
Change from baseline in the CLN2 CRS LX at 12, 18 and 24 Months
Change from baseline in frequency, duration and type of seizures recorded in the Caregiver Seizure Diary at 12, 18 and 24 Months
Change from baseline in the CLN2 Disease Movement Disorder Inventory 12, 18 and 24 Months
Change from baseline in the Pediatric Quality of Life Inventory (PedsQL) Generic Core Scale at 12, 18 and 24 Months

Endpoints to evaluate the effect of Construct III on cognitive function, activities of daily living, adaptive behavior, seizure activity, brain imaging, retinal anatomy, social functioning and quality of life include:
Use of antiepileptic treatments over time
Change in neurodevelopmental parameters of adaptive, cognitive, motor, language and behavioral function over time, including the following:
   Vineland Adaptive Behavior Scales, 3rd Edition (VABS-III)
   Mullen Scales of Early Learning (MSEL)
CNS structural abnormalities assessed by MRI of the brain
Change in retinal anatomy by spectral-domain optical coherence tomography (SD-OCT over time)
Change in QOL measurements over time:
   CLN2 Disease-based QOL Assessment
   Pediatric Quality of Life Inventory Family Impact Module (PedsQL-FIM)
Change in Clinician Global Impression of Severity (Cl-GIS) over time
Change in Clinician Global Impression of Change (Cl-GIC) over time
Changes in gait parameters as captured on the GAITRite

Pharmacodynmamic endpoints include antibodies to adeno-associated virus serotype 9 (AAV9) and TPP1 in CSF and serum, and TPP1 expression in CSF and serum.

### 7.7.3 Immunosuppressive Therapy

Given the immunogenic potential of the vector and transgenic TPP1, immunosuppression will be implemented. Immunosuppressive therapies that may be administered include Corticosteroids (e.g., methylprednisolone), Tacrolimus, and Sirolimus.

### (a) Corticosteroids

Methylprednisolone 10 mg/kg IV once over at least 30 minutes on Day 1 before study intervention administration (maximum of 500 mg). Premedication with acetaminophen and an antihistamine is optional and at the discretion of the investigator.

Oral prednisolone starting at 0.5 mg/kg daily on Day 2 with gradual tapering and discontinuation by Week 12:
0.5 mg/kg/day from Day 2 to end of Week 2
0.35 mg/kg/day from Week 3 to 4
0.2 mg/kg/day from Week 5 to 8
0.1 mg/kg/day from Week 9 to 12.

### (b) Tacrolimus

0.05 mg/kg twice daily (BID) by mouth (PO) Day 2 to Week 32 with target blood level of 2 to 4 ng/mL. Tapering over 8 weeks between Week 24 and Week 32:
Week 24: decrease dose by approximately 50%
Week 28: decrease dose by approximately 50%
Week 32: discontinue tacrolimus

### (c) Sirolimus

A loading dose of 1 mg/m² every 4 hours×3 doses on Day −2. From Day −1 until Week 48: sirolimus 0.5 mg/m²/day divided in BID dosing with target blood level of 1 to 3 ng/mL. Week 48: discontinue sirolimus

The doses of sirolimus and tacrolimus will be adjusted to maintain blood levels in the target range. Dose adjustments for tacrolimus and sirolimus will be performed by a clinical pharmacist. Participants will continue on the new maintenance dose for at least 7 to 14 days before further dosage adjustment with concentration monitoring.

### (d) Other

The following treatments will also be administered to participants to decrease risk of immunosuppression:
Pneumocystis carinii pneumonia prophylaxis: 5 mg/kg trimethoprim/sulfamethoxazole 3 times a week from Day −2 to Week 48. Alternatives to trimethoprim/sulfamethoxazole can include pentamidine, dapsone, and atovaquone.
Antifungal prophylaxis if the absolute neutrophil count is <500 μL.

### 7.8 Example 8: An Expanded CLN2 Clinical Rating Scale Motor (CLN2 CRS-MX) Improves the Evaluation on Ambulatory Function

In late infantile neuronal ceroid lipofuscinosis 2 (CLN2) disease, the rapid progression of the disease was originally quantified using the Hamburg CLN2 scale. The Hamburg motor domain was subsequently adapted into the CLN2 Clinical Rating Scale Motor (CLN2 CRS-M) and used to quantify loss of motor function in the natural disease course compared to the treatment response on cerliponase alfa intraventricular enzyme replacement therapy (ERT). The CLN2 disease impairments includes ataxia, hypotonicity, hypertonicity, and weakness. In children, however, the evolving phenotype on ERT is less perceptible because the disease progression for children is slower.

The aim of this study was to provide a broader and more granular measurement, termed CLN2-CRS-MX, to assess impacts of CLN2 disease impairments on ambulatory capacity.

### 7.8.1 Methods

The derivation of items was based on an identification of key CLN2 disease concepts of interest.

The key concepts were identified from a targeted literature review, clinician expert interviews, two virtual caregiver focus groups and ongoing biweekly meetings for one year with 6 CLN2 clinician experts.

The clinician interviews and caregiver focus groups discussed the key symptoms and impacts of CLN2 specifically related to motor function, differences in disease progression in ERT-treated and ERT-naïve patients, and how to improve the granularity of motor function assessment in the CLN2 CRS to make it more useful for assessing treatment benefit in a clinical trial.

The iterative developmental process included pilot application and numerous item revisions. Tracking matrices were used to document all scale iterations and the rationale for changes.

The inter-rater reliability, i.e. the level of agreement, between the clinicians was calculated as percent agreement across 4 raters.

Assessments were administered and scored by a primary clinician and also independently scored by an observer clinician.

Each assessment was videotaped and scored independently by two additional clinicians.

Thirty patients aged 20 months to 16 years (mean 7.8 years) were each scored by 4 clinicians on the CLN2 CRS-MX scale. They were all currently receiving ERT.

### 7.8.2 Results

The study developed a detailed administrative, scoring, and training manual of the CLN2 CRS-MX scale.

The study developed a typical ambulation task for determining symptom progression. Generally, clinicians and caregivers characterized CLN2 disease by a marked progression in symptoms that deleteriously impact ambulation. Truncal hypotonia, extremity hypertonicity and ataxia were commonly reported symptoms that impacted motor function. As symptoms worsen, an increase in the need for assistance, for example, holding a hand, hands or trunk for balance, was reported, until patients were no longer able to walk. In this study, clinicians supported expansion of the 10-step ambulatory task on the original CLN2 CRS-M, to an ambulatory task that extended the distance walked, considered ability to safely stop, graded a change in direction, and measured the level of assistance that was necessary to ambulate.

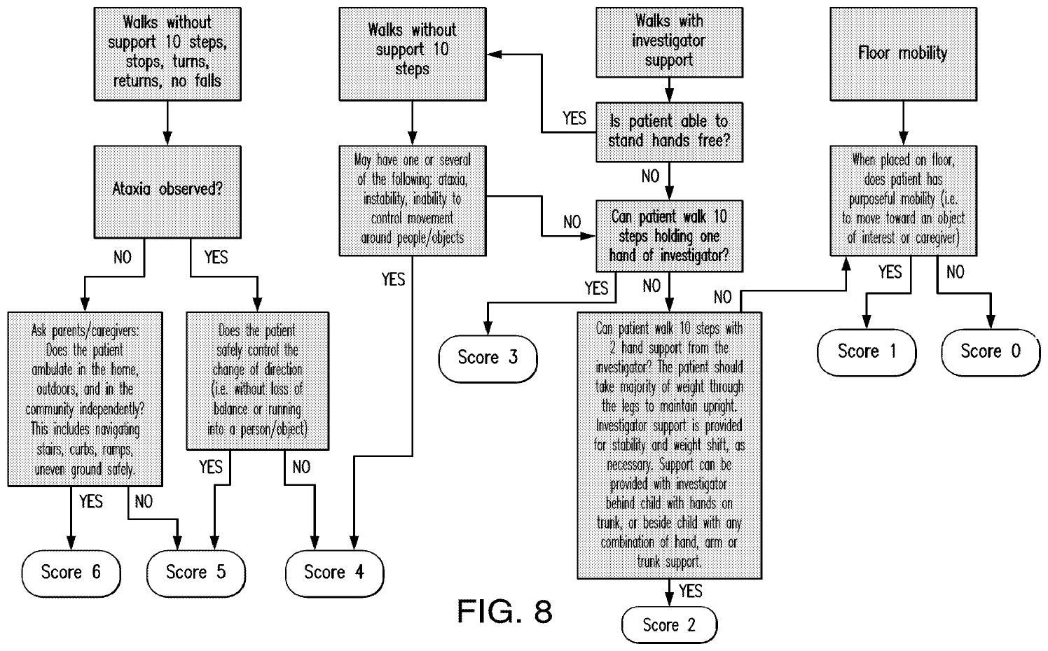

Table 3 provides an outline of the rating criteria and comparisons to the original CLN2 CRS-M. FIG. 8 outlines the CLN2 CRS-MX scoring flow chart to determine the mobility rating.

TABLE 3
CLN2 CRS-M and CLN2 CRS-MX Rating Criteria.

| CLN2 | CLN2 CRS-M | CLN2 | |
|---|---|---|---|
| CRS-M | Rating | CRS-MX | |
| Level | Criteria | Level | CLN2 CRS-MX Rating Criteria |
| A | Grossly | 6 | Normal gait, no ataxia, |
| | normal | | no pathologic falls. Ambulates in |
| | gait. No | | the home, outdoors and in |
| | prominent | | community independently. |
| | ataxia, no | | Child is able to navigate |
| | pathologic | | uneven ground safely, without |
| | falls | | falling (stairs, curbs, ramps etc.) |
| 2 | Independent | 5 | Independent gait as defined by |
| | gait, as | | ability to walk forward without |
| | defined | | support 10 steps, stops and turn |
| | by ability to | | 180° and return. Ataxia may be |
| | walk without | | present but can safely change |
| | support for 10 | | direction without loss of balance or |
| | steps. Will | | running into a person or object |
| | have obvious | 4 | Independent gait defined as ability |
| | instability | | to walk forward without support |
| | and may have | | for 10 steps. May have obvious |
| | intermittent | | instability or ataxia and decreased |
| | falls | | ability to control movement |
| | | | around people or objects |
| 1 | No unaided | 3 | Walks forward 10 |
| | gait. | | steps with one-hand support |
| | Requires | 2 | Walks forward 10 steps |
| | external | | with two-hand support. The |
| | assistance | | patient should take the |
| | to walk | | majority of weight through the |
| | or can | | legs to maintain upright. Support |
| | crawl only | | is provided for stability and |
| | | | weight shift as necessary. Support |
| | | | can be provided behind the child |
| | | | with hands on the trunk or beside |
| | | | the child with any combination of |
| | | | hand, arm or trunk support |
| | | 1 | Floor Mobility: when placed |
| | | | on floor, patient has |
| | | | purposeful mobility to |
| | | | move to an object of interest. |
| | | | Mobility can include crawling, |
| | | | scooting or rolling |
| 0 | Immobile, can | 0 | Can no longer walk, |
| | no longer walk | | scoot, roll or crawl |
| | or crawl | | |

In the inter-rater agreement, the level of agreement between the clinicians for the CLN2 CRS-MX was 1.0, indicating complete/perfect agreement (see Landis, JR, Koch, GG. An application of hierarchical kappa-type statistics in the assessment of majority agreement among multiple observers, Biometrics, 1977. Level of agreement range 0.81-0.99).

### 7.8.3 Conclusions

The CLN2 CRS-MX provided more granularity, improved item relevance, and increased the number of response options in the measurement of CLN2 disease's impact on ambulatory capacity.

The scale was designed for children with CLN2, but may have relevance for other neuromuscular or neurodegenerative disorders that present with similar disease impairments.

### 7.9 Example 9: An Expanded CLN2 Clinical Rating Scale Language (CLN2 CRS-LX) Improves the Evaluation on Language Function

The loss of language function in the natural CLN2 disease course compared to the treatment response on ERT was quantified using the CLN2 Clinical Rating Scale Language (CLN2 CRS-L). However, children's disease progression and phenotype evolution in ERT treatment is slower and less perceptible. The aim of this study was to provide a broader and more granular measurement, termed CLN2 CRS-LX, to capture changes in expressive language and non-verbal communication competencies.

### 7.9.1 Methods

The derivation of items was based on an identification of key CLN2 disease concepts of interest.

The key CLN2 disease concepts were identified from a targeted literature review [not specified in the materials], clinician expert interviews, two virtual caregiver focus groups and ongoing biweekly meetings for one year with 6 CLN2 clinician experts.

The clinician interviews and caregiver focus groups discussed the key symptoms and impacts of CLN2 specifically related to language function, differences in disease progression in ERT-treated and ERT-naïve patients and how to improve the granularity of language function assessment in the CLN2 CRS to make it more useful for assessing treatment benefit in a clinical trial.

The iterative developmental process included pilot application and numerous item revisions. Tracking matrices were used to document all scale iterations and the rationale for changes.

The inter-rater reliability, i.e. the level of agreement, between the clinicians was calculated as percent agreement across 4 raters.

Assessments were administered and scored by a primary clinician and also independently scored by an observer clinician.

Each assessment was videotaped and scored independently by two additional clinicians.

Thirty patients aged 20 months to 16 years (mean 7.8 years) were each scored by 4 clinicians on the CLN2 CRS-MX. They were all currently receiving ERT.

### 7.9.2 Results

The study developed a detailed administrative, scoring and training manual of the CLN2 CRS-LX. The manual contains specific guidance on use of prompts to determine expressive language and non-verbal communication competencies.

The CLN2 CRS-LX measured a child's expressive language ability to communicate wants and needs, including babbling, vocabulary and phrase development, and non-intelligible vocalizations and gestures. Generally, clinicians and caregivers reported that children with CLN2 experience progressive language loss that impacted daily activities. They reported a peak word count of 20-100 words at an age range from 2 to 3.5 years and that children with CLN2 used a range of communication strategies including single and double-word phrases, non-intelligible vocalizations and gestures. In this study, clinicians supported the development of an expanded CLN2 CRS for language that differentiated functional expectations by age, included more response options, and considered vocabulary and phrase development, vocalizations and gestures.

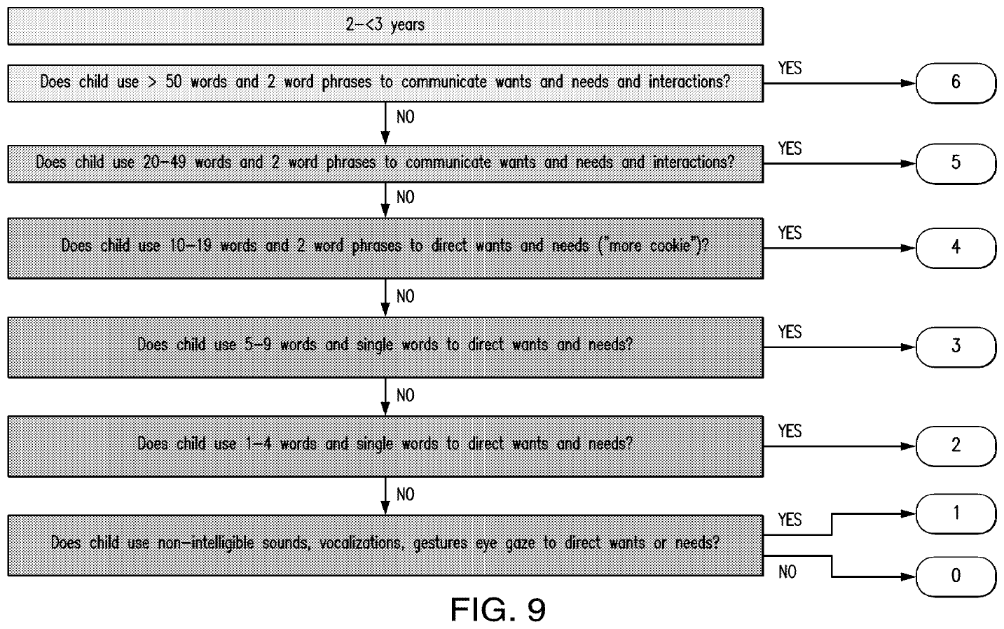

An outline of the rating criteria and comparisons to the original CLN2 CRS-L are provided in Tables 4 and 5. Scoring flowcharts were developed for each CLN2 CRS-LX age category (1 to <2 years, 2 to <3 years and 3 years). FIG. 9 shows the 2 to <3 Year flowchart as an example.

TABLE 4
CLN2 CRS-L Rating Criteria.

| CLN2 CRS-L | |
|---|---|
| Level | CLN2 CRS-L Rating Criteria |
| 3 | Apparently normal language. |
| | Intelligible and grossly age- |
| | appropriate. No decline noted yet |
| 2 | Language has become recognizably |
| | abnormal: some intelligible words |
| | may form short sentences to convey |
| | concepts, requests or needs. |
| | This score signifies a decline from |
| | a previous level of ability (from |
| | the individual maximum reached |
| | by the child) |
| 1 | Hardly understandable. Few intelligible words |
| 0 | No intelligible words or vocalizations |

TABLE 5
CLN2 CRS-LX Rating Criteria

| CLN2 | CLN2 | CLN2 | CLN2 |
|---|---|---|---|
| CRS- | CRS-LX | CRS-LX | CRS-LX |
| MX | Rating | Rating | Rating |
| Level | Criteria | Criteria | Criteria |
| 6 | 1 to <2 years | 2 to <3 years | ≥3 years |
| | Language is normal | Child uses | Child uses 100+ |
| | based on age criteria | ≥50 words | words and |
| | for words, consonant | and 2 word | 3-4 word |
| | vowel combinations | phrases to | phrases to |
| | and gestures | communicate | communicate |
| | 1-2 words at 12 m | wants, | wants, |
| | 8 words by 15 m | needs and | needs and |
| | 15 words by 18 m | interactions | interactions |
| | Uses consonant vowel | | |
| | combinations | | |
| | (ex. baba, mimi) | | |
| | Points or gestures | | |
| | (waves bye bye, | | |
| | shakes head for no, | | |
| | pushes away object, | | |
| | reaches to be picked up | | |
| 5 | Child says less words | Child uses 20-49 | Child uses |
| | than criteria for age. | words and at least | 50-99 words and |
| | Child uses at least | 2 word phrases to | at least 3 word |
| | 2 consonant | communicate | phrases to |
| | vowel combinations | wants, needs and | communicate |
| | and gestures | interactions | wants, needs and |
| | | | interactions |
| 4 | Child uses a | Child uses | Child uses 20-49 |
| | consonant vowel | 10-19 words | words and at least |
| | combination and | and at least 2 | 2 word phrases |
| | point, gesture or back | word phrases | to direct |
| | and forth eye gaze | to direct wants | wants, needs and |
| | | and needs | interactions |
| 3 | Child babbles (vowel | Child uses 5-9 | Child uses 5-19 |
| | sounds) and uses | words and single | words and single |
| | gesture or back | words to direct | words to direct |
| | and forth eye gaze | wants and needs | wants and needs |
| 2 | Child vocalizes mood | Child uses | Child uses |
| | such as pleasure, | 1-4 words | 1-4 words |
| | displeasure, eagerness | and at least | and at least |
| | or satisfaction | single words | single words |
| | in response to | to direct wants | to direct wants |
| | any of the following; | and needs | and needs |
| | social interaction, | | |
| | initiation or | | |
| | interruption of play | | |
| 1 | Child uses cry or | Child uses | Child |
| | non-intelligible | non-intelligible | uses non- |
| | sounds to direct | sounds, | intelligible sounds, |
| | wants and needs | vocalizations, | vocalizations, |
| | | gestures or | gestures or eye |
| | | eye gaze to direct | gaze to direct |
| | | wants and needs | wants and needs |
| 0 | Child does not | Child does not | Child does not |
| | use vocalization | use vocalizations | use |
| | or gestures | or gestures to | vocalizations |
| | to communicate | communicate | or gestures to |
| | wants or needs | wants | communicate |
| | | and needs | wants and needs |

In the inter-rater agreement, the level of agreement between the clinicians for the CLN2 CRS-LX was 0.933, indicating almost perfect agreement (see Landis, JR, Koch, GG. An application of hierarchical kappa-type statistics in the assessment of majority agreement among multiple observers, Biometrics, 1977. Level of agreement range 0.81-0.99).

### 7.9.3 Conclusions

The CLN2 CRS-LX provided more granularity, improved item relevance, and increased the number of response options in the measurement of CLN2 disease's impact on expressive language and non-verbal communication competencies. The scales was designed specifically for children with CLN2, but may have relevance for other neuromuscular or neurodegenerative disorders that present with similar disease impairments.

### 7.10 Example 10: A Single Dose Intrathecal (IT) Pharmacokinetic/Pharmacodynamic Study in Cynomolgus Monkeys

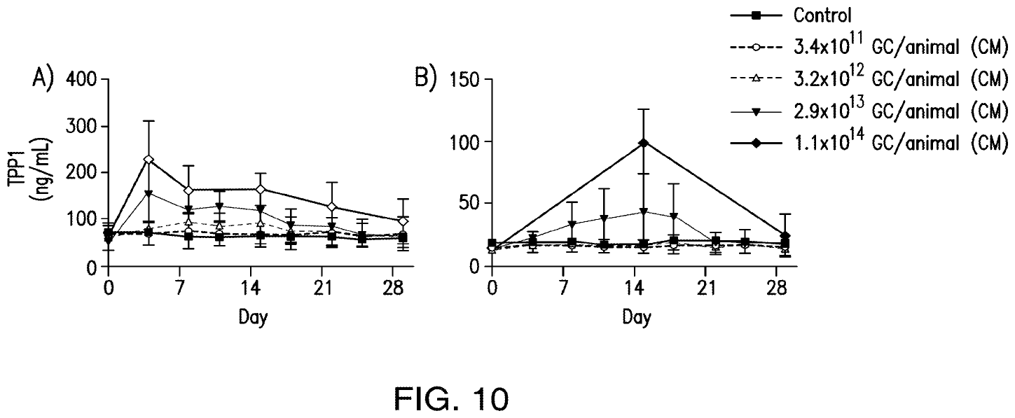

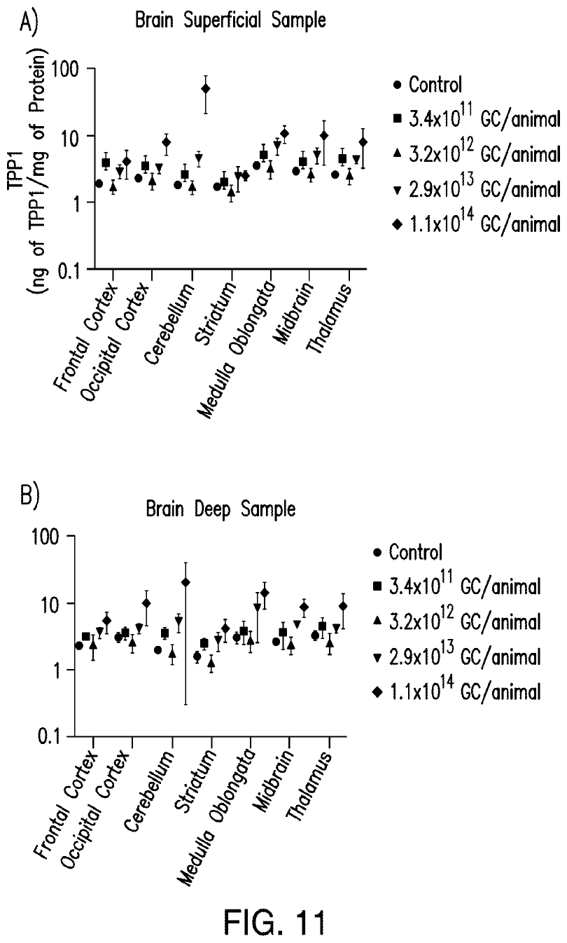

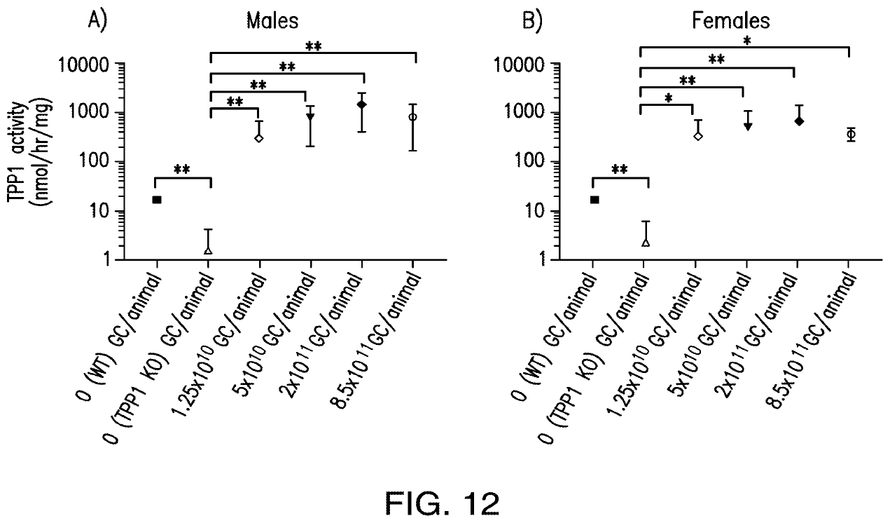

This Example is a continuation of the study reported in Example 3. In the 4-week pharmacodynamic and toxicity study in cynomolgus monkeys with Construct III, samples were collected from the brain (two 4-mm round samples, one superficial and one deep, of frontal cortex, occipital cortex, cerebellum, striatum, medulla oblongata, midbrain and thalamus), spinal cord (1-cm segment of cervical, thoracic and lumbar sections), and the associated nerve roots and ganglia (DRG), eye (left), heart (left ventricle), kidney (left), liver (left lateral lobe), lung (left caudal), proximal sciatic nerve, lymph nodes (inguinal, mandibular and mesenteric), ovary (left) or testis. The eye tissue was further dissected into retina/choroid and sclera. Groups of cynomolgus monkeys (2 males and 2 females/dose) were administered Construct III (AAV9.hCLN2) via a single cisterna magna puncture (CM) at doses of 0, 3.1×10¹³ or 1.1×10¹⁴ genome copies (GC)/animal. At 3.1×10¹³ GC/animals, Construct III was prepared using three different methods. An additional group of animals (n=2/sex/group) was administered AAV9 with a null vector at dose of 2.89×10¹³ GC/animal via a single CM administration. At the end of the study, animals were euthanized on Day 30. Overall, vector DNA levels were above the lower limit of quantification in almost all brain regions, spinal cord and DRG tissues, proximal sciatic nerve and other peripheral tissues collected from animals administered Construct III or null vector on Day 30. The biodistribution results can be found in FIG. 6, FIG. 7, and Table 6. Increases in TPP1 concentration in the serum and cerebrospinal fluid (CSF) were observed (FIG. 10). There were dose-related increases in TPP1 concentration in the brain (FIG. 11) and spinal cord at 4 weeks. A decline in TPP1 concentration in the serum and CSF was associated with immunogenicity observed in these animals.

Groups of cynomolgus monkeys (n=3/group or n=4/group) were administered Construct III (AAV9.hCLN2) via injection into the cisterna magna (CM) at doses of 3.4×10¹¹, 3.2×10¹², 2.9×10¹³ GC/animal or 1.1×10¹⁴ GC/animal. At necropsy, two tissue punches were collected for analysis by qPCR from either the deep (>3 mm; D) or superficial (<3 mm deep; S) areas of frontal cortex, striatum, thalamus, midbrain, occipital cortex, medulla oblongata and cerebellum. Mean and standard deviations are shown. BLQ values were treated as 50.0 copies/μg DNA in the calculation of mean.

Groups of cynomolgus monkeys (n=3/dose) were administered Construct III (AAV9.hCLN2) via injection into the cisterna magna (CM) or via IT-lumbar (IT-L) at a dose of 3.2×10¹² GC/animal. At necropsy, two tissue punches were collected for analysis by qPCR from either the deep (>3 mm; D) or superficial (<3 mm deep; S) areas of frontal cortex, striatum, thalamus, midbrain, occipital cortex, medulla oblongata and cerebellum. Mean and standard deviations are shown. BLQ values were treated as 50.0 copies/pg DNA in the calculation of mean.

TABLE 6
Summary of Vector DNA Biodistribution in cynomolgus monkeys
administered with Construct III (AAV9.hCLN2) via injection
into the cisterna magna (CM) or via IT-lumbar (IT-L)

| | | Dose (GC/animal) | | | | |
|---|---|---|---|---|---|---|
| | | 3.2 × 10¹² | 3.4 × 10¹¹ | 3.2 × 10¹² | 2.9 × 10¹³ | 1.1 × 10¹⁴ |
| | | (IT-L) | (CM) | (CM) | (CM) | (CM) |
| | | Vector DNA copies/μg DNA | | | | |
| Frontal cortex | Superficial | 7.57E+04 | 6.44E+03 | 6.01E+04 | 5.27E+04 | 1.55E+05 |
| | Deep | 3.09E+05 | 2.79E+02 | 1.95E+03 | 4.22E+03 | 5.50E+04 |
| Striatum | Superficial | 4.86E+02 | 3.61E+01 | 1.01E+03 | 1.30E+03 | 4.34E+03 |
| | Deep | 6.59E+02 | 1.95E+02 | 7.88E+03 | 7.65E+03 | 3.57E+04 |
| Thalamus | Superficial | 3.77E+02 | 3.33E+01 | 3.55E+02 | 1.83E+03 | 1.42E+04 |
| | Deep | 4.05E+03 | 9.08E+01 | 9.24E+01 | 3.50E+03 | 2.91E+03 |
| Midbrain | Superficial | 2.82E+04 | 3.60E+02 | 7.97E+02 | 3.31E+03 | 3.18E+04 |
| | Deep | 7.12E+03 | 4.18E+03 | 4.01E+04 | 4.29E+04 | 2.92E+04 |
| Occipital | Superficial | 1.62E+05 | 1.32E+03 | 1.06E+05 | 7.41E+04 | 1.14E+05 |
| Cortex | Deep | 1.24E+04 | 1.16E+03 | 2.79E+04 | 8.75E+03 | 3.95E+04 |
| Medulla | Superficial | 4.90E+05 | 3.56E+02 | 6.27E+04 | 5.08E+04 | 3.18E+04 |
| Oblongata | Deep | 2.00E+03 | 1.18E+03 | 1.39E+04 | 6.27E+03 | 2.23E+04 |
| Cerebellum | Superficial | 1.58E+04 | 1.78E+03 | 1.24E+04 | 2.40E+04 | 5.22E+03 |
| | Deep | 6.09E+03 | 7.43E+02 | 6.09E+03 | 3.14E+04 | 5.52E+04 |
| Spinal Cord | Cervical | 2.81E+05 | 1.36E+04 | 4.93E+02 | 1.61E+05 | 4.64E+04 |
| | Thoracic | 1.26E+05 | 1.18E+04 | 3.65E+02 | 1.91E+05 | 1.51E+05 |
| | Lumbar | 9.68E+03 | 1.36E+05 | 3.71E+05 | 1.55E+05 | 2.12E+05 |

| | | | | | |
|---|---|---|---|---|---|
| Liver | 7.20E+06 | 7.20E+06 | 2.02E+06 | 6.30E+06 | 5.28E+07 |
| Trigeminal ganglion | 5.79E+04 | 5.79E+04 | 5.84E+04 | 5.22E+05 | NA |
| Sciatic nerve | 2.32E+04 | 2.32E+04 | 7.87E+03 | 2.72E+04 | 2.30E+03 |

BLQ values were treated as 50.0 copies/μg DNA in the calculation of mean.
CM = cisterna magna; IT-L = intrathecal-lumbar; NA = Not collected.

### 7.11 Example 11: A Single Dose Pharmacology Study in TPP1m1J KO Mice

This Example is a continuation of the study in Example 4. The objective of this study was to evaluate the pharmacology (clinical signs, neuropathology and survival) of Construct III in TPP1^{m1J} KG mice following a single ICV dose. At the end of the study, additional anatomic pathology evaluation of the spinal cord was conducted in surviving animals. Groups of TPP1^{m1J} KG mice (9-10/sex/group; 4-5 weeks old) were administered a single ICV injection (5 μL) of Construct III at doses of 0 (vehicle), 1.25×10¹⁰, 5.0×10¹⁰, 2.0×10¹¹, and 8.5×10¹¹ GC/animal. Animals were genotyped prior to allocation and at the end of the study.

Endpoints evaluated in this study included: mortality, clinical observations, body weight, neurobehavioral observations (predose, Week 8, and Week 16), TPP1 activity, anti-TPP1 antibody analysis, gross necropsy findings, organ weights, and neuropathology.

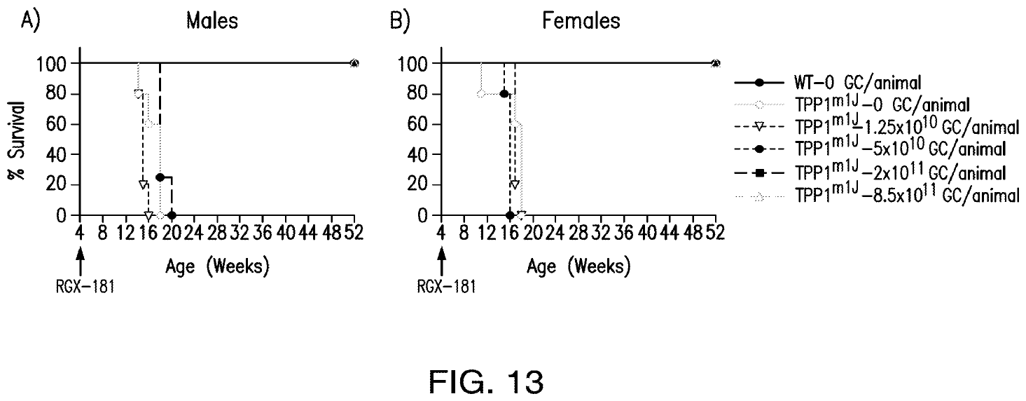

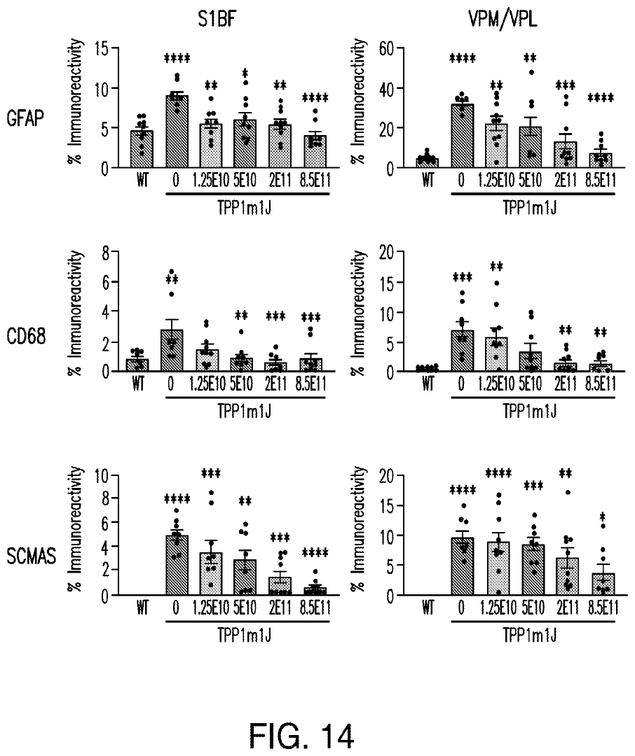

Essentially, a clear effect of Construct III on neurobehavior was not observed because a dose response was not observed or control knock-out animals did not survive to be assessed at later time points. In this study, there were no Construct III-related adverse findings in surviving mice up until 52 weeks at the minimum effective dose for survival (8.5×10¹¹ GC/animal). A single intracerebroventricular (ICV) administration led to expression of hTPP1 (FIG. 12), increased survival (FIG. 13), decreased astrocytosis (GFAP), microglial activation (CD68) and SCMAS in the brain and spinal cord after 9 weeks (FIG. 14). Doses of 1.25×10¹⁰ and 5.0×10¹⁰ GC/animal did not appear to increase the survival of TPP1^{m1J} KO mice when compared to the untreated TPP1^{m1J} KO mice. At 2.0×10¹¹ GC/animal, it was not possible to determine whether or not Construct III prolonged survival as 4/5 males and 2/5 females were heterozygous for the TPP1 gene when genotyped at the end of the study due to supplier error. At this dose, the one confirmed male TPP1^{m1J} KO mouse survived until the end of the study. In all animals receiving Construct III at 8.5×10¹¹ GC/animal, there was 100% survival in both males and females to the scheduled necropsy of 52 weeks. Increased lifespan was also observed at 3×10¹¹ GC/animal (ICV).

There were no macroscopic findings at the Week 9 interval in any animals. At 52 Weeks, a mass on the liver was observed at 2×10¹¹ GC/animal (1/5 males; heterozygous) and 8.5×10¹¹ GC/animal (3/5 males and 1/5 females TPP1^{m1J} KO mice). In the liver, hepatocellular adenoma (1/5 males and 1/5 females at 8.5×10¹¹ GC/animal and 1/5 males at 2×10¹¹ GC/animal), hepatocyte necrosis (3/5 males at 8.5×10¹¹ GC/animal and 1/5 males at 2×10¹¹ GC/animal), hepatocyte hyperplasia (4/5 males at 8.5×10¹¹ GC/animal and 1/2 females at 2×10¹¹ GC/animal) and an increased severity of hepatocyte vacuolation were observed. In the 52-Week cohort, microscopic changes were observed in dorsal root ganglia and spinal nerve roots following administration of Construct III. In the dorsal root ganglia, neuronal vacuolation (minimal to marked) was noted in mice at ≥5.0×10¹⁰ GC/animal and increased cellularity (likely of glial cells, minimal to moderate) and axonal dystrophy/swelling (minimal to mild) were seen in mice at ≥2.0×10¹¹ GC/animal. Degeneration (minimal to moderate) and axonal dystrophy/swelling (minimal to mild) in the spinal roots were noted in mice at ≥5.0×10¹⁰ GC/animal. Due to a lack of vehicle control TPP1^{m1J} KO mice surviving to the 52-week necropsy, it was not possible to evaluate livers of TPP1^{m1J} KO mice not administered Construct III; thus, differentiation between CONSTRUCT III-related and phenotype-related lesions was not possible.

### 8. INCORPORATION BY REFERENCE

All published documents cited in this specification are incorporated herein by reference. Similarly, the SEQ ID NOs which are referenced herein and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

### 9. SEQUENCES

### Table of Sequences:

| Name | Sequence |
|---|---|
| TPP1 protein | MGLQACLLGLFALILSGKCSYSPEPDQRRTLPPGWVSLGRADPEEELS |
| sequence | LTFALRQQNVERLSELVQAVSDPSSPQYGKYLTLENVADLVRPSPLT |
| (UniProtKB/ | LHTVQKWLLAAGAQKCHSVITQDFLTCWLSIRQAELLLPGAEFHHY |
| Swiss-Prot | VGGPTETHVVRSPHPYQLPQALAPHVDFVGGLHRFPPTSSLRQRPEP |
| Accession No. | QVTGTVGLHLGVTPSVIRKRYNLTSQDVGSGTSNNSQACAQFLEQYF |
| 014773-1) | HDSDLAQFMRLFGGNFAHQASVARVVGQQGRGRAGIEASLDVQYL |
| | MSAGANISTWVYSSPGRHEGQEPFLQWLMLLSNESALPHVHTVSYG |
| | DDEDSLSSAYIQRVNTELMKAAARGLTLLFASGDSGAGCWSVSGRH |
| | QFRPTFPASSPYVTTVGGTSFQEPFLITNEIVDYISGGGFSNVFPRPSYQ |
| | EEAVTKFLSSSPHLPPSSYFNASGRAYPDVAALSDGYWVVSNRVPIP |
| | WVSGTSASTPVFGGILSLINEHRILSGRPPLGFLNPRLYQQHGAGLFD |
| | VTRGCHESCLDEEVEGQGFCSGPGWDPVTGWGTPNFPALLKTLLNP |
| | (SEQ ID NO: 1) |
| Native TPP1 | atgggactcc aagcctgcct cctagggctc tttgccctca tcctctctgg caaatgcagttacagcccgg |
| Coding | agcccgacca gcggaggacg ctgcccccag gctgggtgtc cctgggccgtgcggaccctg |
| Sequence | aggaagagct gagtctcacc tttgccctga gacagcagaa tgtggaaagactctcggagc |
| | tggtgcaggc tgtgtcggat cccagctctc ctcaatacgg aaaatacctgaccctagaga atgtggctga |
| | tctggtgagg ccatccccac tgaccctcca cacggtgcaaaaatggctct tggcagccgg |
| | agcccagaag tgccattctg tgatcacaca ggactttctg acttgctggc tgagcatccg |
| | acaagcagag ctgctgctcc ctggggctga gtttcatcac tatgtgggag gacctacgga |
| | aacccatgtt gtaaggtccc cacatcccta ccagcttcca caggccttgg ccccccatgt ggactttgtg |
| | gggggactgc accgttttcc cccaacatca tccctgaggc aacgtcctga gccgcaggtg |
| | acagggactg taggcctgca tctgggggta accccctctg tgatccgtaa gcgatacaac |
| | ttgacctcac aagacgtggg ctctggcacc agcaataaca gccaagcctg tgcccagttc |
| | ctggagcagt atttccatga ctcagacctg gctcagttca tgcgcctctt cggtggcaac tttgcacatc |
| | aggcatcagt agcccgtgtg gttggacaac agggccgggg ccgggccggg attgaggcca |
| | gtctagatgt gcagtacctg atgagtgctg gtgccaacat ctccacctgg gtctacagta gccctggccg |
| | gcatgaggga caggagccct tcctgcagtg gctcatgctg ctcagtaatg agtcagccct |
| | gccacatgtg catactgtga gctatggaga tgatgaggac tccctcagca gcgcctacat |
| | ccagcgggtc aacactgagc tcatgaaggc tgccgctcgg ggtctcaccc tgctcttcgc |
| | ctcaggtgac agtggggccg ggtgttggtc tgtctctgga agacaccagt tccgccctac cttccctgcc |
| | tccagcccct atgtcaccac agtgggaggc acatccttcc aggaaccttt cctcatcaca aatgaaattg |
| | ttgactatat cagtggtggt ggcttcagca atgtgttccc acggccttca taccaggagg aagctgtaac |
| | gaagttcctg agctctagcc cccacctgcc accatccagt tacttcaatg ccagtggccg tgcctaccca |
| | gatgtggctg cactttctga tggctactgg gtggtcagca acagagtgcc cattccatgg gtgtccggaa |
| | cctcggcctc tactccagtg tttgggggga tcctatcctt gatcaatgag cacaggatcc ttagtggccg |
| | cccccctctt ggctttctca acccaaggct ctaccagcag catggggcag gactctttga tgtaacccgt |
| | ggctgccatg agtcctgtct ggatgaagag gtagagggcc agggtttctg ctctggtcct |
| | ggctgggatc ctgtaacagg ctggggaaca cccaacttcc cagctttgct gaagactcta ctcaacccct |
| | gac (SEQ ID NO: 2) |
| Codon- | atgggactgc aggcctgtct gctgggactg ttcgccctga tcctgagcgg caagtgcagc |
| optimized | tacagccccg agcccgacca gagaagaaca ctgcctccag gctgggtgtc cctgggcaga |
| TPP1 coding | gctgaccctg aagaggaact gagcctgacc ttcgccctgc ggcagcagaa cgtggaaaga |
| sequence | ctgagcgagc tggtgcaggc cgtgtccgat cctagcagcc ctcagtacgg caagtacctg |
| | accctggaaa acgtggccga cctcgtgcgg cctagccctc tgacactgca caccgtgcag |
| | aagtggctgc tggctgccgg cgctcagaaa tgccactccg tgatcaccca ggactttctg |
| | acctgttggc tgagcatccg gcaggccgaa ctgctgctgc ctggggccga gtttcaccac |
| | tatgtgggcg gacccaccga gacacatgtc gtgcgcagcc cacaccctta ccagctgcca |
| | caggctctgg cccctcacgt ggactttgtg ggaggcctgc acagattccc cccaaccagc |
| | agcctgagac agaggcctga gccacaagtg accggcacag tgggcctgca tctgggcgtg |
| | acacctagcg tgatccggaa gcggtacaac ctgaccagcc aggatgtggg cagcggcacc |
| | agcaacaata gccaggcctg cgcccagttc ctggaacagt acttccacga cagcgatctg |
| | gcccagttca tgcggctgtt cggcggcaac ttcgcacatc aggctagcgt ggccagagtc |
| | gtgggccagc agggaagagg cagagccgga attgaggcct ccctggacgt gcagtacctg |
| | atgagcgctg gcgccaacat cagcacctgg gtgtacagca gccccggcag acacgagggc |
| | caggaacctt ttctgcagtg gctgatgctg ctgagcaacg agagcgccct gcctcatgtg |
| | cacacagtgt cctacggcga cgacgaggac agcctgagca gcgcctacat ccagagagtg |
| | aacaccgagc tgatgaaggc cgctgccagg ggactgaccc tgctgtttgc ctctggcgat |
| | agcggagccg gctgttggag tgtgtcaggc cggcaccagt tcagacccac ctttcctgcc |
| | agctccccct acgtgacaac cgtgggcggc acctcctttc aggaaccctt cctgatcacc |
| | aacgagatcg tggactacat cagcggcgga ggcttcagca acgtgttccc cagacccagc |
| | taccaggaag aggccgtgac caagttcctg tcctccagcc ctcatctgcc ccccagctcc tacttcaacg |
| | ccagcggcag agcctaccca gatgtggccg ctctgtccga cggctactgg gtggtgtcca |
| | acagagtgcc catcccttgg gtgtccggca caagcgccag cacccctgtg tttggcggca |
| | tcctgtccct gatcaacgag cacagaatcc tgtccggcag accccccctg ggcttcctga |
| | accctagact gtaccagcag cacggcgctg gcctgttcga tgtgaccaga ggctgccacg |
| | agagctgcct ggacgaggaa gtggaaggcc agggcttctg ttctggccct ggctgggatc |
| | ctgtgaccgg atggggcacc cctaacttcc ccgccctgct gaaaacactg ctgaacccct gat (SEQ |
| | ID NO: 3) |
| TPP1 protein | MRLFGGNFAHQASVARVVGQQGRGRAGIEASLDVQYLMSAGANIST |
| sequence | WVYSSPGRHEGQEPFLQWLMLLSNESALPHVHTVSYGDDEDSLSSA |
| (UniProtKB/ | YIQRVNTELMKAAARGLTLLFASGDSGAGCWSVSGRHQFRPTFPASS |
| Swiss-Prot | PYVTTVGGTSFQEPFLITNEIVDYISGGGFSNVFPRPSYQEEAVTKFLS |
| Accession No. | SSPHLPPSSYFNASGRAYPDVAALSDGYWVVSNRVPIPWVSGTSAST |
| O14773-2) | PVFGGILSLINEHRILSGRPPLGFLNPRLYQQHGAGLFDVTRGCHESCL |
| | DEEVEGQGFCSGPGWDPVTGWGTPNFPALLKTLLNP (SEQ ID NO: 4) |
| Exemplary | tcgaggacgg ggtgaactac gcctgaggat ccgatctttt tccctctgcc aaaaattatg gggacatcat |
| production | gaagcccctt gagcatctga cttctggcta ataaaggaaa tttattttca ttgcaatagt gtgttggaat |
| plasmid which | tttttgtgtc tctcactcgg aagcaattcg ttgatctgaa tttcgaccac ccataatacc cattaccctg |
| includes the | gtagataagt agcatggcgg gttaatcatt aactacaagg aacccctagt gatggagttg gccactccct |
| TPP1 | ctctgcgcgc tcgctcgctc actgaggccg ggcgaccaaa ggtcgcccga cgcccgggct |
| encoding | ttgcccgggc ggcctcagtg agcgagcgag cgcgcagcct taattaacct aattcactgg ccgtcgtttt |
| sequence | acaacgtcgt gactgggaaa accctggcgt tacccaactt aatcgccttg cagcacatcc ccctttcgcc |
| | agctggcgta atagcgaaga ggcccgcacc gatcgccctt cccaacagtt gcgcagcctg |
| | aatggcgaat gggacgcgcc ctgtagcggc gcattaagcg cggcgggtgt ggtggttacg |
| | cgcagcgtga ccgctacact tgccagcgcc ctagcgcccg ctcctttcgc tttcttccct tcctttctcg |
| | ccacgttcgc cggctttccc cgtcaagctc taaatcgggg gctcccttta gggttccgat ttagtgcttt |
| | acggcacctc gaccccaaaa aacttgatta gggtgatggt tcacgtagtg ggccatcgcc |
| | ctgatagacg gtttttcgcc ctttgacgtt ggagtccacg ttctttaata gtggactctt gttccaaact |
| | ggaacaacac tcaaccctat ctcggtctat tcttttgatt tataagggat tttgccgatt tcggcctatt |
| | ggttaaaaaa tgagctgatt taacaaaaat ttaacgcgaa ttttaacaaa atattaacgc ttacaattta |
| | ggtggcactt ttcggggaaa tgtgcgcgga acccctattt gtttattttt ctaaatacat tcaaatatgt |
| | atccgctcat gagacaataa ccctgataaa tgcttcaata atattgaaaa aggaagagta tgagtattca |
| | acatttccgt gtcgccctta ttcccttttt tgcggcattt tgccttcctg tttttgctca cccagaaacg |
| | ctggtgaaag taaaagatgc tgaagatcag ttgggtgcac gagtgggtta catcgaactg |
| | gatctcaaca gcggtaagat ccttgagagt tttcgccccg aagaacgttt tccaatgatg agcactttta |
| | aagttctgct atgtggcgcg gtattatccc gtattgacgc cgggcaagag caactcggtc |
| | gccgcataca ctattctcag aatgacttgg ttgagtactc accagtcaca gaaaagcatc ttacggatgg |
| | catgacagta agagaattat gcagtgctgc cataaccatg agtgataaca ctgcggccaa cttacttctg |
| | acaacgatcg gaggaccgaa ggagctaacc gcttttttgc acaacatggg ggatcatgta |
| | actcgccttg atcgttggga accggagctg aatgaagcca taccaaacga cgagcgtgac |
| | accacgatgc ctgtagcaat ggcaacaacg ttgcgcaaac tattaactgg cgaactactt actctagctt |
| | cccggcaaca attaatagac tggatggagg cggataaagt tgcaggacca cttctgcgct |
| | cggcccttcc ggctggctgg tttattgctg ataaatctgg agccggtgag cgtgggtctc gcggtatcat |
| | tgcagcactg gggccagatg gtaagccctc ccgtatcgta gttatctaca cgacggggag |
| | tcaggcaact atggatgaac gaaatagaca gatcgctgag ataggtgcct cactgattaa gcattggtaa |
| | ctgtcagacc aagtttactc atatatactt tagattgatt taaaacttca tttttaattt aaaaggatct |
| | aggtgaagat cctttttgat aatctcatga ccaaaatccc ttaacgtgag ttttcgttcc actgagcgtc |
| | agaccccgta gaaaagatca aaggatcttc ttgagatcct ttttttctgc gcgtaatctg ctgcttgcaa |
| | acaaaaaaac caccgctacc agcggtggtt tgtttgccgg atcaagagct accaactctt tttccgaagg |
| | taactggctt cagcagagcg cagataccaa atactgttct tctagtgtag ccgtagttag gccaccactt |
| | caagaactct gtagcaccgc ctacatacct cgctctgcta atcctgttac cagtggctgc tgccagtggc |
| | gataagtcgt gtcttaccgg gttggactca agacgatagt taccggataa ggcgcagcgg |
| | tcgggctgaa cggggggttc gtgcacacag cccagcttgg agcgaacgac ctacaccgaa |
| | ctgagatacc tacagcgtga gctatgagaa agcgccacgc ttcccgaagg gagaaaggcg |
| | gacaggtatc cggtaagcgg cagggtcgga acaggagagc gcacgaggga gcttccaggg |
| | ggaaacgcct ggtatcttta tagtcctgtc gggtttcgcc acctctgact tgagcgtcga tttttgtgat |
| | gctcgtcagg ggggcggagc ctatggaaaa acgccagcaa cgcggccttt ttacggttcc |
| | tggccttttg ctggcctttt gctcacatgt tctttcctgc gttatcccct gattctgtgg ataaccgtat |
| | taccgccttt gagtgagctg ataccgctcg ccgcagccga acgaccgagc gcagcgagtc |
| | agtgagcgag gaagcggaag agcgcccaat acgcaaaccg cctctccccg cgcgttggcc |
| | gattcattaa tgcagctggc acgacaggtt tcccgactgg aaagcgggca gtgagcgcaa |
| | cgcaattaat gtgagttagc tcactcatta ggcaccccag gctttacact ttatgcttcc ggctcgtatg |
| | ttgtgtggaa ttgtgagcgg ataacaattt cacacaggaa acagctatga ccatgattac gccagattta |
| | attaaggcct taattaggct gcgcgctcgc tcgctcactg aggccgcccg ggcaaagccc |
| | gggcgtcggg cgacctttgg tcgcccggcc tcagtgagcg agcgagcgcg cagagaggga |
| | gtggccaact ccatcactag gggttccttg tagttaatga ttaacccgcc atgctactta tctaccaggg |
| | taatggggat cctctagaac tatagctagt cgacattgat tattgactag ttattaatag taatcaatta |
| | cggggtcatt agttcatagc ccatatatgg agttccgcgt tacataactt acggtaaatg gcccgcctgg |
| | ctgaccgccc aacgaccccc gcccattgac gtcaataatg acgtatgttc ccatagtaac |
| | gccaataggg actttccatt gacgtcaatg ggtggactat ttacggtaaa ctgcccactt ggcagtacat |
| | caagtgtatc atatgccaag tacgccccct attgacgtca atgacggtaa atggcccgcc tggcattatg |
| | cccagtacat gaccttatgg gactttccta cttggcagta catctacgta ttagtcatcg ctattaccat |
| | ggtcgaggtg agccccacgt tctgcttcac tctccccatc tcccccccct ccccaccccc aattttgtat |
| | ttatttattt tttaattatt ttgtgcagcg atgggggcgg gggggggggg ggggcgcgcg |
| | ccaggcgggg cgggggcggg cgaggggcgg ggcggggcga ggcggagagg tgcggcggca |
| | gccaatcaga gcggcgcgct ccgaaagttt ccttttatgg cgaggcggcg gcggcggcgg |
| | ccctataaaa agcgaagcgc gcggcgggcg gggagtcgct gcgacgctgc cttcgccccg |
| | tgccccgctc cgccgccgcc tcgcgccgcc cgccccggct ctgactgacc gcgttactcc |
| | cacaggtgag cgggcgggac ggcccttctc ctccgggctg taattagcgc ttggtttaat gacggcttgt |
| | ttcttttctg tggctgcgtg aaagccttga ggggctccgg gagggccctt tgtgcgggg |
| | gagcggctcg gggggtgcgt gcgtgtgtgt gtgcgtgggg agcgccgcgt gcggctccgc |
| | gctgcccggc ggctgtgagc gctgcgggcg cggcgcgggg ctttgtgcgc tccgcagtgt |
| | gcgcgagggg agcgcggccg ggggcggtgc cccgcggtgc ggggggggct gcgaggggaa |
| | caaaggctgc gtgcggggtg tgtgcgtggg ggggtgagca gggggtgtgg gcgcgtcggt |
| | cgggctgcaa ccccccctgc acccccctcc ccgagttgct gagcacggcc cggcttcggg |
| | tgcggggctc cgtacggggc gtggcgcggg gctcgccgtg ccgggcgggg ggtggcggca |
| | ggtgggggtg ccgggcgggg cggggccgcc tcgggccggg gagggctcgg gggaggggcg |
| | cggcggcccc cggagcgccg gcggctgtcg aggcgcggcg agccgcagcc attgcctttt |
| | atggtaatcg tgcgagaggg cgcagggact tcctttgtcc caaatctgtg cggagccgaa |
| | atctgggagg cgccgccgca ccccctctag cgggcgcggg gcgaagcggt gcggcgccgg |
| | caggaaggaa atgggcgggg agggccttcg tgcgtcgccg cgccgccgtc cccttctccc |
| | tctccagcct cggggctgtc cgcgggggga cggctgcctt cgggggggac ggggcagggc |
| | ggggttcggc ttctggcgtg tgaccggcgg ctctagagcc tctgctaacc atgttcatgc cttcttcttt |
| | ttcctacagc tcctgggcaa cgtgctggtt attgtgctgt ctcatcattt tggcaaagaa ttcacgcgtg |
| | ccaccatggg actgcaggcc tgtctgctgg gactgttcgc cctgatcctg agcggcaagt |
| | gcagctacag ccccgagccc gaccagagaa gaacactgcc tccaggctgg gtgtccctgg |
| | gcagagctga ccctgaagag gaactgagcc tgaccttcgc cctgcggcag cagaacgtgg |
| | aaagactgag cgagctggtg caggccgtgt ccgatcctag cagccctcag tacggcaagt |
| | acctgaccct ggaaaacgtg gccgacctcg tgcggcctag ccctctgaca ctgcacaccg |
| | tgcagaagtg gctgctggct gccggcgctc agaaatgcca ctccgtgatc acccaggact |
| | ttctgacctg ttggctgagc atccggcagg ccgaactgct gctgcctggg gccgagtttc accactatgt |
| | gggcggaccc accgagacac atgtcgtgcg cagcccacac ccttaccagc tgccacaggc |
| | tctggcccct cacgtggact ttgtgggagg cctgcacaga ttccccccaa ccagcagcct |
| | gagacagagg cctgagccac aagtgaccgg cacagtgggc ctgcatctgg gcgtgacacc |
| | tagcgtgatc cggaagcggt acaacctgac cagccaggat gtgggcagcg gcaccagcaa |
| | caatagccag gcctgcgccc agttcctgga acagtacttc cacgacagcg atctggccca |
| | gttcatgcgg ctgttcggcg gcaacttcgc acatcaggct agcgtggcca gagtcgtggg |
| | ccagcaggga agaggcagag ccggaattga ggcctccctg gacgtgcagt acctgatgag |
| | cgctggcgcc aacatcagca cctgggtgta cagcagcccc ggcagacacg agggccagga |
| | accttttctg cagtggctga tgctgctgag caacgagagc gccctgcctc atgtgcacac agtgtcctac |
| | ggcgacgacg aggacagcct gagcagcgcc tacatccaga gagtgaacac cgagctgatg |
| | aaggccgctg ccaggggact gaccctgctg tttgcctctg gcgatagcgg agccggctgt |
| | tggagtgtgt caggccggca ccagttcaga cccacctttc ctgccagctc cccctacgtg |
| | acaaccgtgg gcggcacctc ctttcaggaa cccttcctga tcaccaacga gatcgtggac |
| | tacatcagcg gcggaggctt cagcaacgtg ttccccagac ccagctacca ggaagaggcc |
| | gtgaccaagt tcctgtcctc cagccctcat ctgcccccca gctcctactt caacgccagc ggcagagcct |
| | acccagatgt ggccgctctg tccgacggct actgggtggt gtccaacaga gtgcccatcc |
| | cttgggtgtc cggcacaagc gccagcaccc ctgtgtttgg cggcatcctg tccctgatca |
| | acgagcacag aatcctgtcc ggcagacccc ccctgggctt cctgaaccct agactgtacc |
| | agcagcacgg cgctggcctg ttcgatgtga ccagaggctg ccacgagagc tgcctggacg |
| | aggaagtgga aggccagggc ttctgttctg gccctggctg ggatcctgtg accggatggg |
| | gcacccctaa cttccccgcc ctgctgaaaa cactgctgaa cccctgatga c (SEQ ID NO: 5) |
| AAV9 Capsid | MAADGYLPDWLEDNLSEGIREWWALKPGAPQPKANQQHQDNARG |
| | LVLPGYKYLGPGNGLDKGEPVNAADAAALEHDKAYDQQLKAGDNP |
| | YLKYNHADAEFQERLKEDTSFGGNLGRAVFQAKKRLLEPLGLVEEA |
| | AKTAPGKKRPVEQSPQEPDSSAGIGKSGAQPAKKRLNFGQTGDTESV |
| | PDPQPIGEPPAAPSGVGSLTMASGGGAPVADNNEGADGVGSSSGNW |
| | HCDSQWLGDRVITTSTRTWALPTYNNHLYKQISNSTSGGSSNDNAYF |
| | GYSTPWGYFDFNRFHCHFSPRDWQRLINNNWGFRPKRLNFKLFNIQ |
| | VKEVTDNNGVKTIANNLTSTVQVFTDSDYQLPYVLGSAHEGCLPPFP |
| | ADVFMIPQYGYLTLNDGSQAVGRSSFYCLEYFPSQMLRTGNNFQFSY |
| | EFENVPFHSSYAHSQSLDRLMNPLIDQYLYYLSKTINGSGQNQQTLK |
| | FSVAGPSNMAVQGRNYIPGPSYRQQRVSTTVTQNNNSEFAWPGASS |
| | WALNGRNSLMNPGPAMASHKEGEDRFFPLSGSLIFGKQGTGRDNVD |
| | ADKVMITNEEEIKTTNPVATESYGQVATNHQSAQAQAQTGWVQNQ |
| | GILPGMVWQDRDVYLQGPIWAKIPHTDGNFHPSPLMGGFGMKHPPP |
| | QILIKNTPVPADPPTAFNKDKLNSFITQYSTGQVSVEIEWELQKENSK |
| | RWNPEIQYTSNYYKSNNVEFAVNTEGVYSEPRPIGTRYLTRNL (SEQ |
| | ID NO: 6) |
| AAV9 Capsid | atggctgccg atggttatct tccagattgg ctcgaggaca accttagtga aggaattcgc gagtggtggg |
| coding | ctttgaaacc tggagcccct caacccaagg caaatcaaca acatcaagac aacgctcgag |
| sequence | gtcttgtgct tccgggttac aaataccttg gacccggcaa cggactcgac aagggggagc |
| | cggtcaacgc agcagacgcg gcggccctcg agcacgacaa ggcctacgac cagcagctca |
| | aggccggaga caacccgtac ctcaagtaca accacgccga cgccgagttc caggagcggc |
| | tcaaagaaga tacgtctttt gggggcaacc tcgggcgagc agtcttccag gccaaaaaga |
| | ggcttcttga acctcttggt ctggttgagg aagcggctaa gacggctcct ggaaagaaga |
| | ggcctgtaga gcagtctcct caggaaccgg actcctccgc gggtattggc aaatcgggtg |
| | cacagcccgc taaaaagaga ctcaatttcg gtcagactgg cgacacagag tcagtcccag |
| | accctcaacc aatcggagaa cctcccgcag ccccctcagg tgtgggatct cttacaatgg |
| | cttcaggtgg tggcgcacca gtggcagaca ataacgaagg tgccgatgga gtgggtagtt |
| | cctcgggaaa ttggcattgc gattcccaat ggctggggga cagagtcatc accaccagca |
| | cccgaacctg ggccctgccc acctacaaca atcacctcta caagcaaatc tccaacagca |
| | catctggagg atcttcaaat gacaacgcct acttcggcta cagcaccccc tgggggtatt ttgacttcaa |
| | cagattccac tgccacttct caccacgtga ctggcagcga ctcatcaaca acaactgggg |
| | attccggcct aagcgactca acttcaagct cttcaacatt caggtcaaag aggttacgga caacaatgga |
| | gtcaagacca tcgccaataa ccttaccagc acggtccagg tcttcacgga ctcagactat cagctcccgt |
| | acgtgctcgg gtcggctcac gagggctgcc tcccgccgtt cccagcggac gttttcatga ttcctcagta |
| | cgggtatctg acgcttaatg atggaagcca ggccgtgggt cgttcgtcct tttactgcct ggaatatttc |
| | ccgtcgcaaa tgctaagaac gggtaacaac ttccagttca gctacgagtt tgagaacgta cctttccata |
| | gcagctacgc tcacagccaa agcctggacc gactaatgaa tccactcatc gaccaatact tgtactatct |
| | ctcaaagact attaacggtt ctggacagaa tcaacaaacg ctaaaattca gtgtggccgg |
| | acccagcaac atggctgtcc agggaagaaa ctacatacct ggacccagct accgacaaca |
| | acgtgtctca accactgtga ctcaaaacaa caacagcgaa tttgcttggc ctggagcttc ttcttgggct |
| | ctcaatggac gtaatagctt gatgaatcct ggacctgcta tggccagcca caaagaagga |
| | gaggaccgtt tctttccttt gtctggatct ttaatttttg gcaaacaagg aactggaaga gacaacgtgg |
| | atgcggacaa agtcatgata accaacgaag aagaaattaa aactactaac ccggtagcaa |
| | cggagtccta tggacaagtg gccacaaacc accagagtgc ccaagcacag gcgcagaccg |
| | gctgggttca aaaccaagga atacttccgg gtatggtttg gcaggacaga gatgtgtacc |
| | tgcaaggacc catttgggcc aaaattcctc acacggacgg caactttcac ccttctccgc |
| | tgatgggagg gtttggaatg aagcacccgc ctcctcagat cctcatcaaa aacacacctg |
| | tacctgcgga tcctccaacg gccttcaaca aggacaagct gaactctttc atcacccagt attctactgg |
| | ccaagtcagc gtggagatcg agtgggagct gcagaaggaa aacagcaagc gctggaaccc |
| | ggagatccag tacacttcca actattacaa gtctaataat gttgaatttg ctgttaatac tgaaggtgta |
| | tatagtgaac cccgccccat tggcaccaga tacctgactc gtaatctgta a (SEQ ID NO: 7) |
| Exemplary | ctgcgcgctc gctcgctcac tgaggccgcc cgggcaaagc ccgggcgtcg ggcgaccttt |
| sequence of | ggtcgcccgg cctcagtgag cgagcgagcg cgcagagagg gagtggccaa ctccatcact |
| an expression | aggggttcct tgtagttaat gattaacccg ccatgctact tatctaccag ggtaatgggg atcctctaga |
| cassette | actatagcta gtcgacattg attattgact agttattaat agtaatcaat tacggggtca ttagttcata |
| | gcccatatat ggagttccgc gttacataac ttacggtaaa tggcccgcct ggctgaccgc |
| | ccaacgaccc ccgcccattg acgtcaataa tgacgtatgt tcccatagta acgccaatag ggactttcca |
| | ttgacgtcaa tgggtggact atttacggta aactgcccac ttggcagtac atcaagtgta tcatatgcca |
| | agtacgcccc ctattgacgt caatgacggt aaatggcccg cctggcatta tgcccagtac atgaccttat |
| | gggactttcc tacttggcag tacatctacg tattagtcat cgctattacc atggtcgagg tgagccccac |
| | gttctgcttc actctcccca tctccccccc ctccccaccc ccaattttgt atttatttat tttttaatta |
| | ttttgtgcag cgatgggggc gggggggggg ggggggcgcg cgccaggcgg ggcggggcgg |
| | ggcgaggggc ggggcggggc gaggcggaga ggtgcggcgg cagccaatca gagcggcgcg |
| | ctccgaaagt ttccttttat ggcgaggcgg cggcggcggc ggccctataa aaagcgaagc |
| | gcgcggcggg cggggagtcg ctgcgacgct gccttcgccc cgtgccccgc tccgccgccg |
| | cctcgcgccg cccgccccgg ctctgactga ccgcgttact cccacaggtg agcgggggg |
| | acggcccttc tcctccgggc tgtaattagc gcttggttta atgacggctt gtttcttttc tgtggctgcg |
| | tgaaagcctt gaggggctcc gggagggccc tttgtgcggg gggagcggct cggggggtgc |
| | gtgcgtgtgt gtgtgcgtgg ggagcgccgc gtgcggctcc gcgctgcccg gcggctgtga |
| | gcgctgcggg cgcggcgcgg ggctttgtgc gctccgcagt gtgcgcgagg ggagcgcggc |
| | cgggggcggt gccccgcggt gcgggggggg ctgcgagggg aacaaaggct gcgtgcgggg |
| | tgtgtgcgtg ggggggtgag cagggggtgt gggcgcgtcg gtcgggctgc aaccccccct |
| | gcacccccct ccccgagttg ctgagcacgg cccggcttcg ggtgcggggc tccgtacggg |
| | gcgtggcgcg gggctcgccg tgccgggcgg ggggtggcgg caggtggggg tgccggggg |
| | ggcggggccg cctcgggccg gggagggctc gggggagggg cgcggcggcc cccggagcgc |
| | cggcggctgt cgaggcgcgg cgagccgcag ccattgcctt ttatggtaat cgtgcgagag |
| | ggcgcaggga cttcctttgt cccaaatctg tgcggagccg aaatctggga ggcgccgccg |
| | caccccctct agcgggcgcg gggcgaagcg gtgcggcgcc ggcaggaagg aaatgggcgg |
| | ggagggcctt cgtgcgtcgc cgcgccgccg tccccttctc cctctccagc ctcggggctg |
| | tccgcggggg gacggctgcc ttcggggggg acggggcagg gcggggttcg gcttctggcg |
| | tgtgaccggc ggctctagag cctctgctaa ccatgttcat gccttcttct ttttcctaca gctcctgggc |
| | aacgtgctgg ttattgtgct gtctcatcat tttggcaaag aattcacgcg tgccaccatg ggactgcagg |
| | cctgtctgct gggactgttc gccctgatcc tgagcggcaa gtgcagctac agccccgagc |
| | ccgaccagag aagaacactg cctccaggct gggtgtccct gggcagagct gaccctgaag |
| | aggaactgag cctgaccttc gccctgcggc agcagaacgt ggaaagactg agcgagctgg |
| | tgcaggccgt gtccgatcct agcagccctc agtacggcaa gtacctgacc ctggaaaacg |
| | tggccgacct cgtgcggcct agccctctga cactgcacac cgtgcagaag tggctgctgg |
| | ctgccggcgc tcagaaatgc cactccgtga tcacccagga ctttctgacc tgttggctga |
| | gcatccggca ggccgaactg ctgctgcctg gggccgagtt tcaccactat gtgggcggac |
| | ccaccgagac acatgtcgtg cgcagcccac acccttacca gctgccacag gctctggccc |
| | ctcacgtgga ctttgtggga ggcctgcaca gattcccccc aaccagcagc ctgagacaga |
| | ggcctgagcc acaagtgacc ggcacagtgg gcctgcatct gggcgtgaca cctagcgtga |
| | tccggaagcg gtacaacctg accagccagg atgtgggcag cggcaccagc aacaatagcc |
| | aggcctgcgc ccagttcctg gaacagtact tccacgacag cgatctggcc cagttcatgc |
| | ggctgttcgg cggcaacttc gcacatcagg ctagcgtggc cagagtcgtg ggccagcagg |
| | gaagaggcag agccggaatt gaggcctccc tggacgtgca gtacctgatg agcgctggcg |
| | ccaacatcag cacctgggtg tacagcagcc ccggcagaca cgagggccag gaaccttttc |
| | tgcagtggct gatgctgctg agcaacgaga gcgccctgcc tcatgtgcac acagtgtcct |
| | acggcgacga cgaggacagc ctgagcagcg cctacatcca gagagtgaac accgagctga |
| | tgaaggccgc tgccagggga ctgaccctgc tgtttgcctc tggcgatagc ggagccggct |
| | gttggagtgt gtcaggccgg caccagttca gacccacctt tcctgccagc tccccctacg |
| | tgacaaccgt gggcggcacc tcctttcagg aacccttcct gatcaccaac gagatcgtgg |
| | actacatcag cggcggaggc ttcagcaacg tgttccccag acccagctac caggaagagg |
| | ccgtgaccaa gttcctgtcc tccagccctc atctgccccc cagctcctac ttcaacgcca |
| | gcggcagagc ctacccagat gtggccgctc tgtccgacgg ctactgggtg gtgtccaaca |
| | gagtgcccat cccttgggtg tccggcacaa gcgccagcac ccctgtgttt ggcggcatcc |
| | tgtccctgat caacgagcac agaatcctgt ccggcagacc ccccctgggc ttcctgaacc |
| | ctagactgta ccagcagcac ggcgctggcc tgttcgatgt gaccagaggc tgccacgaga |
| | gctgcctgga cgaggaagtg gaaggccagg gcttctgttc tggccctggc tgggatcctg |
| | tgaccggatg gggcacccct aacttccccg ccctgctgaa aacactgctg aacccctgat |
| | gactcgagga cggggtgaac tacgcctgag gatccgatct ttttccctct gccaaaaatt atggggacat |
| | catgaagccc cttgagcatc tgacttctgg ctaataaagg aaatttattt tcattgcaat agtgtgttgg |
| | aattttttgt gtctctcact cggaagcaat tcgttgatct gaatttcgac cacccataat acccattacc |
| | ctggtagata agtagcatgg cgggttaatc attaactaca aggaacccct agtgatggag ttggccactc |
| | cctctctgcg cgctcgctcg ctcactgagg ccgggcgacc aaaggtcgcc cgacgcccgg |
| | gctttgcccg ggcggcctca gtgagcgagc gagcgcgcag (SEQ ID NO: 8) |

The following information is provided for sequences containing free text under numeric identifier <223> or <213>.

| SEQ ID NO: | |
|---|---|
| (containing | |
| free text) | Free text under <223> or <213> |
| 3 | <223> constructed sequence |
| 5 | <223> constructed sequence |
| 6 | <213> adeno-associated virus 9 |
| 7 | <213> adeno-associated virus 9 |
| 8 | <223> constructed sequence |

What is claimed is:

1. A method of treating CLN2 disease due to TPP1 deficiency in a subject comprising administering to the central nervous system of the subject in need thereof 1.25×10¹¹ or 4.5×10¹¹ genome copies per gram brain mass of a recombinant adeno-associated virus (rAAV) into the central nervous system (CNS), wherein said recombinant adeno-associated virus (rAAV) comprises an AAV capsid and a vector genome packaged therein, and wherein said vector genome comprises
(a) an AAV 5′ inverted terminal repeat (ITR) sequence;
(b) a promoter;
(c) a CLN2 coding sequence encoding a human TPP1; and
(d) an AAV 3′ ITR;
wherein the method results in an improvement of symptoms of CLN2 disease.

2. The method of claim 1, wherein the rAAV is administered intracerebroventricularly (ICV) or intracisternally (IC).

3. The method of claim 1 or 2, wherein the brain mass of the subject is derived from the study participant's screening brain MRI.

4. The method of any one of claims 1 to 3, wherein the coding sequence of (c) is a codon optimized human CLN2 set forth in SEQ ID NO: 3.

5. The method of any one of claims 1 to 4, wherein the coding sequence of (c) is SEQ ID NO: 3.

6. The method of any one of claims 1 to 5, wherein the rAAV capsid is an AAV9 or a variant thereof.

7. The method of any one of claims 1 to 6, wherein the promoter is a chicken beta actin (CBA) promoter.

8. The method of any one of claims 1 to 7, wherein the promoter is a hybrid promoter comprising a CBA promoter sequence and cytomegalovirus enhancer elements.

9. The method of any one of claims 1 to 8, wherein the AAV 5′ ITR and/or AAV3′ ITR is from AAV2.

10. The method of any one of claims 1 to 9, wherein the vector genome further comprises a polyA.

11. The method of claim 10, wherein the polyA is a synthetic polyA or from bovine growth hormone (bGH), human growth hormone (hGH), SV40, rabbit β-globin (RGB), or modified RGB (mRGB).

12. The method of any one of claims 1 to 11, wherein the vector genome further comprises an intron.

13. The method of claim 12, wherein the intron is from CBA, human beta globin, IVS2, SV40, bGH, alpha-globulin, beta-globulin, collagen, ovalbumin, or p53.

14. The method of any one of claims 1 to 13, wherein the vector genome further comprises an enhancer.

15. The method of claim 14, wherein the enhancer is a CMV enhancer, an RSV enhancer, an APB enhancer, ABPS enhancer, an alpha mic/bik enhancer, TTR enhancer, en34, ApoE.

16. The method of any one of claims 1 to 15, wherein said method results in a less than 2-category decline in the 6-point combined Motor and Language domains of the CLN2 Clinical Rating Scale within 24 months after administration.

17. The method of any one of claims 1 to 16, wherein said method results in a TPP1 activity in the cerebral spinal fluid of the subject that is at least about 50%, at least about 75%, at least about 80%, at least about 90%, or about the same, or greater than 100% of the biological activity level of the native TPP1 protein, or a natural variant or polymorph thereof which is not associated with disease.

18. The method of any one of claims 1 to 17, wherein said method results in a serum TPP1 activity of said subject that is at least about 50%, at least about 75%, at least about 80%, at least about 90%, or about the same, or greater than 100% of the biological activity level of the native TPP1 protein, or a natural variant or polymorph thereof which is not associated with disease.

19. The method of any one of claims 1 to 18, wherein the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the combined Motor and Language domains of the CLN2 CRS.

20. The method of any one of claims 1 to 19, wherein the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the Language domains of the CLN2 CRS.

21. The method of any one of claims 1 to 20, wherein the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the Motor domains of the CLN2 CRS.

22. The method of any one of claims 1 to 21, wherein the method results in a reduction in the frequency of seizures of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as recorded in the Caregiver Seizure Diary.

23. The method of any one of claims 1 to 22, wherein the method results in a reduction in the duration of seizures of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as recorded in the Caregiver Seizure Diary.

24. The method of any one of claims 1 to 23, wherein the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the Pediatric Quality of Life Inventory Generic Core Scale.

25. The method of any one of claims 1 to 24, wherein the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as measured by the PedsQL Family Impact Module.

26. The method of any one of claims 1 to 25, wherein the method results in a decrease in the use of antiepileptic treatments of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline.

27. The method of any one of claims 1 to 26, wherein the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by the Vineland Adaptive Behavior Scale III.

28. The method of any one of claims 1 to 27, wherein the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by the Mullen Scale of Early Learning.

29. The method of any one of claims 1 to 28, wherein the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by assessing retinal anatomy using Spectral Domain Optical Coherence Tomography (SD-OCT).

30. The method of any one of claims 1 to 29, wherein the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by Clinician Global Impression of Severity.

31. The method of any one of claims 1 to 30, wherein the method results in a clinical improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% compared to baseline as determined by Clinician Global Impression of Change.

32. The method of any one of claims 1 to 31, wherein the method results in an improvement of about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more than 95% in gait parameters compared to baseline as determined by GAITRite.

33. The method of any one of 1 to 32, wherein the method further comprises administering immunosuppressive therapy to the subject.

34. The method of claim 33, wherein the immunosuppressive therapy comprises administering corticosteroids, tacrolimus, and/or sirolimus.

35. The method according to any one of claims 1 to 34, wherein said subject is human.

36. The method of any one of claims 1 to 35, wherein the subject is between 4 months and 6 years of age.

37. The method of any one of claims 1 to 36, wherein the subject has a documented diagnosis of CLN2 disease due to TPP1 deficiency, confirmed by biochemical, molecular, or genetic methods.
